(19) United States
(12) Patent Application Publication
(10) Pub. No.: US 2026/0097022 A1
(43) Pub. Date: Apr. 9, 2026

(54) COMPOUNDS AND USES THEREOF

(71) Applicant: Integrated Biosciences, Inc., San Carlos, CA (US)

(72) Inventors: Felix WONG, San Carlos, CA (US); Satotaka OMORI, San Carlos, CA (US)

(21) Appl. No.: 19/348,275

(22) Filed: Oct. 2, 2025

Related U.S. Application Data
(63) Continuation of application No. PCT/US2024/023426, filed on Apr. 5, 2024.
(60) Provisional application No. 63/457,548, filed on Apr. 6, 2023.
(60) Provisional application No. 63/463,736, filed on May 3, 2023.

(51) Int. Cl. A61K 31/426; A61K 9/00; A61P 43/00; C07D 277/74

(52) U.S. Cl. CPC A61K 31/426; A61K 9/006; A61P 43/00; C07D 277/74

(57) ABSTRACT

The accumulation of senescent cells is associated with aging, inflammation, and cellular dysfunction. Senolytic drugs can alleviate age-related comorbidities by selectively killing senescent cells. Described herein is a screen of 2,352 compounds for senolytic activity in a model of senescence and trained neural networks to predict the senolytic activities of >800,000 molecules. Methods, systems, and algorithms of the disclosure can enrich for structurally diverse compounds with senolytic activity. Compounds of the disclosure can comprise drug-like compounds which selectively target senescent cells across different senescence models, with more favorable medicinal chemistry properties than, and selectivity comparable to, those of a documented senolytics. Molecular docking simulations of compound binding to several senolytic protein targets, combined with time-resolved fluorescence energy transfer experiments, indicate that these compounds act in part by inhibiting Bcl-2, a regulator of cellular apoptosis. Compounds of the present disclosure can decrease senescent cell burden and mRNA expression of senescence-associated genes.

### CROSS-REFERENCE

This application is a continuation of International Application No. PCT/US2024/023426, filed Apr. 5, 2024, which claims the benefit of U.S. Provisional Patent Application Nos. 63/457,548 filed on Apr. 6, 2023; and 63/463,736 filed on May 3, 2023; the entire contents of each of which are incorporated herein by reference.

### BACKGROUND OF THE INVENTION

The accumulation of senescent cells is associated with aging, inflammation, and cellular dysfunction. Senolytic drugs can alleviate age-related comorbidities by selectively killing senescent cells.

### SUMMARY

Provided herein are pharmaceutical compositions comprising a compound of Formula

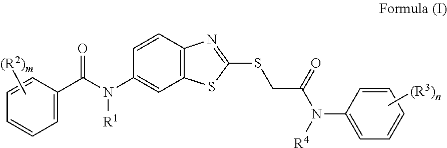

or a pharmaceutically acceptable salt thereof and a pharmaceutically acceptable excipient, wherein:
R¹ and R⁴ are each independently hydrogen or C₁₋₆ alkyl;
each R² and R³ is independently selected from the group consisting of halogen, —OR⁵, —SR⁵, —N(R⁵)₂, —C(O)R⁶, —C(O)N(R⁵)₂, —N(R⁵)C(O)R⁶, —C(O)OR⁵, —OC(O)R⁶, —NO₂, —CN, C₁₋₆ alkyl, C₂₋₆ alkenyl, and C₂₋₆ alkynyl; wherein each C₁₋₆ alkyl, C₂₋₆ alkenyl, and C₂₋₆ alkynyl is optionally substituted with one or more substituents independently selected from halogen, —OR⁵, —SR⁵, —N(R⁵)₂, —C(O)R⁶, —C(O)N(R⁵)₂, —N(R⁵)C(O)R⁶, —C(O)OR⁵, —OC(O)R⁶, —NO₂, ═O, and —CN;
each R⁵ is independently selected from: hydrogen; and C₁₋₆ alkyl optionally substituted with one more substituents independently selected from halogen, —O—C₁₋₆ alkyl, —O—C₁₋₆ haloalkyl, —NH₂—NO₂, ═O, and —CN;
each R⁶ is independently selected from: C₁₋₆ alkyl optionally substituted with one more substituents independently selected from halogen, —O—C₁₋₆ alkyl, —O—C₁₋₆ haloalkyl, —NH₂—NO₂, ═O, and —CN:

m is 0, 1, 2, 3, 4, or 5; and
n is 0, 1, 2, 3, 4, or 5.

In certain embodiments, provided herein is a pharmaceutical composition comprising a compound of Formula (I) above, or a pharmaceutically salt thereof, and a pharmaceutically acceptable excipient, wherein: R¹ and R⁴ are each hydrogen; and each R² and R³ is independently halogen, —O—C₁₋₆ alkyl, C₁₋₆ alkyl, —O—C₁₋₆ haloalkyl, or C₁₋₆ haloalkyl; m is selected from 1 or 2; and n is 0, 1, 2, or 3.

The pharmaceutical composition may, for example, comprise a compound of Formula (I) represented by one of the following structures:

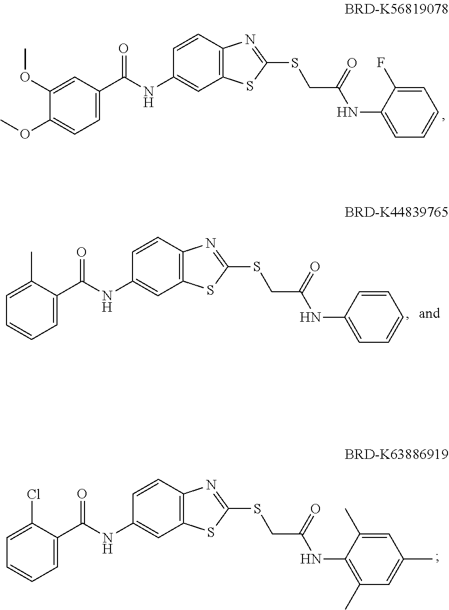 [SMILES: COc1ccc(C(=O)Nc2ccc3nc(SCC(=O)Nc4ccccc4F)sc3c2)cc1OC ; Cc1ccccc1C(=O)Nc1ccc2nc(SCC(=O)Nc3ccccc3)sc2c1 ; Cc1cc(C)c(NC(=O)CSc2nc3ccc(NC(=O)c4ccccc4Cl)cc3s2)c(C)c1]

or a pharmaceutically acceptable salt thereof.

In some embodiments, provided herein is a pharmaceutical composition comprising the compound:

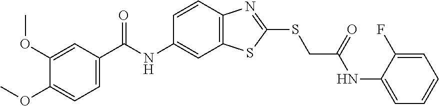 [SMILES: COc1ccc(C(=O)Nc2ccc3nc(SCC(=O)Nc4ccccc4F)sc3c2)cc1OC]

or a pharmaceutically acceptable salt thereof, e.g., for use in treating a disease or disorder (such as a senescence-associated disease or disorder, and/or an age-related disease or disorder).

In another aspect of the present invention provided herein is a compound of Formula (II):

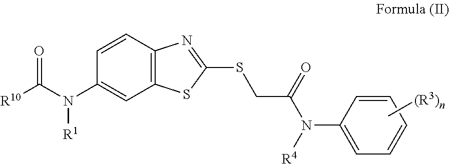

or a pharmaceutically acceptable salt thereof, wherein:
R¹ and R⁴ are each independently hydrogen or C₁₋₆ alkyl;
R¹⁰ is selected from

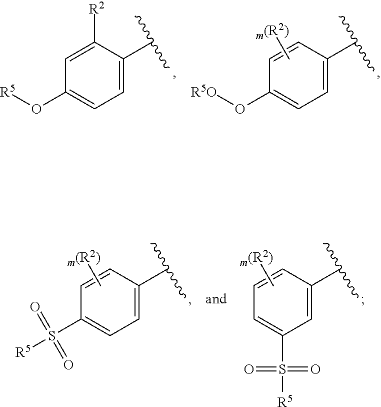

each R² and R³ is independently selected from the group consisting of halogen, —OR⁵, —SR⁵, —N(R⁵)₂, —C(O)R⁶, —C(O)N(R⁵)₂, —N(R⁵)C(O)R⁶, —C(O)OR⁵, —C(O)OR⁵, —OC(O)R⁶, —S(O)₂R⁶, —S(O)₂N(R⁵)₂, —NO₂, —CN, C₁₋₆ alkyl, C₂₋₆ alkenyl, and C₂₋₆ alkynyl; wherein each C₁₋₆ alkyl, C₂₋₆ alkenyl, and C₂₋₆ alkynyl is optionally substituted with one or more substituents independently selected from halogen, —OR⁵, —SR⁵, —N(R⁵)₂, —C(O)R⁶, —C(O)N(R⁵)₂, —N(R⁵)C(O)R⁶, —C(O)OR⁵, —OC(O)R⁶, —NO₂, ═O, and —CN;
each R⁵ is independently selected from: hydrogen; and C₁₋₆ alkyl optionally substituted with one more substituents independently selected from halogen, —O—C₁₋₆ alkyl, —O—C₁₋₆ haloalkyl, —NH₂—NO₂, —O, and —CN;
each R⁶ is independently selected from: C₁₋₆ alkyl optionally substituted with one more substituents independently selected from halogen, —O—C₁₋₆ alkyl, —O—C₁₋₆ haloalkyl, —NH₂, —NO₂, ═O, and —CN;
m is 0, 1, 2, 3, or 4; and
n is 0, 1, 2, 3, 4, or 5.

In another aspect of the present invention, provided herein is a method of treating a disease or disorder in a subject in need thereof, comprising administering to the subject a therapeutically effective amount of the compound of Formula (I), or a pharmaceutically acceptable salt thereof, or a pharmaceutical composition comprising the same. In some embodiments, the disease or disorder is a senescence-associated disease or disorder. In some embodiments, the senescence-associated disease or disorder is an age-related disease or disorder. In still another aspect of the present invention, provided herein is a method of decreasing senescent cell burden and/or mRNA expression of senescence-associated genes in a subject comprising contacting the senescent cell with a senolytic agent provided herein (e.g., a compound according to Formula (I)), or a pharmaceutically acceptable salt thereof, or the pharmaceutical composition comprising the same. In some embodiments, the method comprising contacting the senescent cell with BRD-K56819078. BRD-K44839765. BRD-K3886919, or a pharmaceutically acceptable salt thereof. In some embodiments, the method comprising contacting the senescent cell with a compound of Table 2, or a pharmaceutically acceptable salt thereof.

In another aspect of the present invention, provided herein is a method of treating a disease or disorder in a subject in need thereof, comprising administering to the subject a therapeutically effective amount of the compound of Formula (II), or a pharmaceutically acceptable salt thereof, or a pharmaceutical composition comprising the same. In some embodiments, the disease or disorder is a senescence-associated disease or disorder. In some embodiments, the senescence-associated disease or disorder is an age-related disease or disorder. In still another aspect of the present invention, provided herein is a method of decreasing senescent cell burden and/or mRNA expression of senescence-associated genes in a subject comprising contacting the senescent cell with a senolytic agent provided herein (e.g., a compound according to Formula (II)), or a pharmaceutically acceptable salt thereof, or the pharmaceutical composition comprising the same. In some embodiments, the method comprising contacting the senescent cell with a compound described herein, or a pharmaceutically acceptable salt thereof.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

A better understanding of the features and advantages of the present invention will be obtained by reference to the following detailed description that sets forth illustrative embodiments, in which the principles of the invention are utilized, and the accompanying drawings (also “Figure” or “FIG.” herein), of which:

FIG. 1 shows graph neural networks predict senolytic activity. Panel 1a shows a schematic of the approach where 2,352 compounds, including known senolytics, were screened for senolytic activity, and the data were used to train graph neural networks. Trained models were applied to predict the senolytic activities of 804.959 compounds. Compounds predicted to be active were tested for senolytic activity. Panel 1b shows SA-β-gal staining of vehicle-(0.5% DMSO) and etoposide-treated IMR-90 cells plated on the day before and day of compound addition. Each image represents two biological replicates. Scale bar. 100 μm. Panel 1c shows relative mRNA expression of p16, p21, and KI67 in vehicle-(0.5% DMSO) and etoposide-treated IMR-90 cells on the day of compound addition. Data from three biological replicates are shown, and bars represent average values. Error bars indicate one standard deviation. Two-sided two-sample t-test for differences in mean value: *** p<0.005. Panel 1d shows senolytic screening results for 2.352 compounds at a final concentration of 10 μM. Values show the mean of two biological replicates, and viability measurements are normalized by the interquartile mean of each plate. Active compounds are those for which relative control cell viability was >0.5, relative Snc viability was <0.7, and the ratio of Snc to control cell viability was <0.7. All other compounds are inactive. Three documented senolytics. ABT-737. ABT-263, and A-1331852, are highlighted (large points). Sncs were induced with etoposide, and control cells were treated with vehicle (0.5% DMSO). Panel 1e shows precision-recall curves for 10 Chemprop models trained and tested on the data in (Panel 1d). The dashed curve represents the baseline fraction of active compounds in the training set (1.9%). Light curves and the 95% confidence interval (CI: heavy curve) indicate the variation generated by bootstrapping. AUC, area under the curve. Panel 1f shows rank-ordered prediction scores (PS) of 804.959 compounds, for 20 Chemprop models trained on all the data in (Panel 1d). Panel 1g shows a t-SNE plot of compounds with high and low predicted senolytic activity and the training set shown in (Panel 1d). Validated compounds refer to FIG. 2.

FIG. 2 shows the identification of structurally diverse compounds with senolytic activity. Panel 2a shows cell viability measurements for 266 curated compounds, including 216 high-ranking compounds from the Broad Institute's Drug Repurposing Hub or an extended Broad Institute library and the bottom-ranking 50 compounds from the Drug Repurposing Hub. Values indicate the mean of two biological replicates, and cell viability measurements are normalized with respect to the interquartile mean viability of each cell plate. The final concentration of all compounds was 10 μM. Sncs were treated with etoposide for senescence induction, and control cells were treated with vehicle (0.5% DMSO). Panel 2b shows rank-ordered prediction scores of the 266 curated compounds, with high- and low-ranking compounds separated by the vertical line. All curated compounds found to be active in (Panel 2a) fall below the curve (Snc:control ratio <0.7) within the panel where (Relative Snc viability<0.7) and (Relative control cell viability >0.5) intersect. Panel 2c shows molecular weights and Tanimoto similarity scores of the 25 identified true positive compounds, rank-ordered by increasing molecular weight. For comparison, values for a documented senolytic. ABT-737, are shown. Lipinski-conforming molecular weights are those <500 Da, and lower Tanimoto similarity scores indicate higher structural novelty with respect to the training set.

FIG. 3 shows validation of identified compounds in a model of therapy-induced senescence. Panels 3a-3d show dose-response curves of control and etoposide-treated IMR-90) cells treated with BRD-K20733377 (Panel 3a), BRD-K56819078 (Panel 3b), BRD-K44839765 (Panel 3c), and ABT-737 (Panel 3d) for comparison. Compounds were serially diluted two-fold starting from a final concentration of 50 μM, and 0 μM (1% DMSO vehicle) treatment was included. Cells were treated for 3 days. Cellular viability was determined by the metabolic reduction of resazurin into fluorescent resorufin, and values are normalized by the fluorescence intensities of the average of two untreated samples from the same phenotype: here, a cellular viability of 1 indicates that of either untreated control cells or Sncs. Vehicle treatment could result in cellular viability values <1 due to effects of DMSO on cellular viability. Curves for both control (vehicle-treated) cells, and etoposide-treated Sncs are depicted. Measurements are shown for two biological replicates in each treatment group (open points), and mean viability values (closed points) were fitted to calculate IC₅₀ values. The therapeutic index (TI) is the ratio of IC₅₀ values for vehicle- and etoposide-treated cells. The chemical structure of each compound is shown in the inset of each plot. Panel 3e shows additional structurally diverse active compounds, with the therapeutic index (TI) of each compound indicated for vehicle- and etoposide-treated cells (see also FIG. 11). Panel 3f shows cellular viability measurements for control (untreated) and etoposide-treated IMR-90 cells treated with varying concentrations of BRD-K20733377. BRD-K56819078. BRD-K44839765, and ABT-737. Cells were treated for 3 days. Values shown are normalized to the mean cell viability value for cells treated with vehicle (1% DMSO) for 3 days, such that cell proliferation in the presence of DMSO vehicle between days 0 and 3 is indicated by an increase in cellular viability values. Data from two biological replicates are shown (solid points), and bars represent average values. Solid lines highlight inhibited control cell proliferation induced by treatment with ABT-737.

FIG. 4 shows validation of identified compounds in a model of replicative senescence. Panel 4a shows SA-β-gal staining of early- and late-passage IMR-90 cells plated at times corresponding to the day before and day of compound addition. Each image is representative of two biological replicates. Scale bar. 100 μm. Panel 4b shows relative mRNA expression of p16, p21, and KI67 in early- and late-passage IMR-90 cells harvested on the day of compound addition. Data from three biological replicates are shown (solid points), and bars represent average values. Error bars indicate one standard deviation. Two-sided two-sample/-test for differences in mean value: ** p<0.01. *** p<0.005. Panels 4c-4f show dose-response curves of early- and late-passage IMR-90 cells treated with BRD-K20733377 (Panel 4c), BRD-K56819078 (Panel 4d), BRD-K44839765 (Panel 4e), and ABT-737 (Panel 4f) for comparison. Compounds were serially diluted two-fold starting from a final concentration of 50 μM, and 0 μM (1% DMSO vehicle) treatment was included. Cells were treated for 3 days. Cellular viability was determined by the metabolic reduction of resazurin into fluorescent resorufin, and values are normalized by the fluorescence intensities of the average of two untreated samples from the same phenotype: here, a cellular viability of 1 indicates that of either untreated early- or late-passage cells. Vehicle treatment may result in cellular viability values <1 due to minor effects of DMSO on cellular viability. Curves are shown for both control (early-passage) cells and late-passage Sncs. Measurements are shown for two biological replicates in each treatment group (open points), and mean viability (closed points) were fitted to calculate IC₅₀ values. The therapeutic index (TI) is the ratio of IC₅₀ values for early- and late-passage cells.

FIG. 5 shows molecular docking- and TR-FRET-based identification of Bcl-2 as a potential binding target of compounds of the present dislcosure. Panel 5a shows dopcumented senolytic protein targets in humans. Protein Data Bank (PDB) identifiers used for molecular docking and relevant protein functions are indicated. As controls for the docking simulations, examples of documented binding inhibitors of each protein were identified, and their molecular structures were used for docking. Panel 5b shows schematic of the molecular docking approach. Experimentally-determined protein structures in complex with various inhibitors were curated from the PDB, and protein active sites were determined from the inhibitor-bound conformations. Chemical compounds of interest were represented in 3D and docked in the active site of each protein structure using AutoDock Vina. Binding affinities were then calculated for each protein-ligand pair and used to rank likely binding targets (Panel 5c). Panel 5c shows predicted binding affinities (kcal/mol) for each of the three identified compounds and known inhibitor compounds with each of the known senolytic protein targets in (Panel 5a). Values are from docking simulations involving each potential protein-ligand pair and are representative of 32 runs each. Lower binding affinity values indicate higher predicted binding activity. For each ligand, the protein target with the lowest predicted binding affinity of those tested are highlighted with solid lines. Documented protein-ligand interactions are highlighted (dashed lines). Panel 5d shows molecular docking poses of ABT-737 (positive control: main figure and inset), BRD-K20733377 (inset), BRD-K56819078 (inset), and BRD-K44839765 (inset) with Bcl-2. Indicated amino acid residues are depicted in a stick representation. Panel 5e shows relative Bcl-2 activity in the presence of varying concentrations of BRD-K20733377. BRD-K56819078, BRD-K44839765, and ABT-737, as measured by TR-FRET. Solid points indicate values from individual biological replicates, and bars indicate average values. Unpaired two-sided t-tests for no change in Bcl-2 activity after compound treatment (compared to relative Bcl-2 activity values of 1 arising from vehicle treatment only): * p≤0.05. *** p<0.005.

FIG. 6 shows In vivo efficacy of BRD-K56819078 in an aged mouse model. Panel 6a shows a schematic of the validation experiment. Kidneys were harvested from naïve young and aged mice and tested for senescent cell burden, as measured by SA-β-gal staining and mRNA expression of p16 and p21. Panel 6b shows relative SA-β-gal-positive area in kidney sections of young and aged mice (n 3 mice each, and one kidney per mouse). Data shown are from two fields of view for each kidney, and horizontal lines represent average values. Panel 6c shows relative mRNA expression of p16 and p21 in the kidneys of young and aged mice (n 3 mice each, and one kidney per mouse). Horizontal lines represent average values. One-sided, two-sample permutation test for differences in mean value: * p≤0.05. Panel 6d shows a schematic of the aged mouse experiment. Kidneys were harvested from aged mice treated intraperitoneally six times with vehicle or BRD-K56819078 (25 mg/kg per injection) and tested for senescent cell burden, as measured by SA-β-gal staining and mRNA expression of p16 and p21. Panel 6e shows relative SA-β-gal-positive area in kidney sections of vehicle- and BRD-K56819078-treated aged mice (n 7 mice each, and one kidney per mouse). Data shown are from two fields of view for each kidney, and horizontal lines represent average values. One-sided, two-sample permutation test for differences in mean value: * p≤0.05. Panel 6f shows relative mRNA expression of p16 and p21 in the kidneys of vehicle- and BRD-K56819078-treated aged mice (n 8 mice each, and one kidney per mouse). Horizontal lines represent average values. One-sided, two-sample permutation test for differences in mean value: * p≤0.05.

FIG. 7 shows a timeline of etoposide-induced senescence. Control (DMSO-treated) and senescent (etoposide-treated) cells were treated with test compounds and assayed for cellular viability at the indicated times for compound screening and dose-response experiments.

FIG. 8 shows screening of 2.352 compounds for senolytic activity and validation of four active compounds. Panel a shows cellular viability of vehicle- and etoposide-treated cells after a 3-day course of test compound treatment (10 μM). Values are from two biological replicates, and viability measurements are normalized by the interquartile mean of each cell plate. Active compounds are those for which the relative control cell viability is >0.5, the relative Snc viability is <0.7, and the ratio of Snc to control cell viability is <0.7. All other compounds are inactive. The Pearson's correlation coefficient. R, and two-sided p-value are shown. Panel b shows dose-response curves of control and etoposide-treated IMR-90 cells, treated with each compound shown. Zero μM (1% DMSO vehicle) treatment was included. Values are normalized by the average of two untreated samples from the same phenotype: here, a cellular viability of 1 indicates that of either untreated control cells or Sncs. Curves for both control (vehicle-treated) cells and etoposide-treated Sncs are shown. Measurements are shown for two biological replicates in each treatment group (open points), and mean viability values (closed points) were fitted to calculate IC₅₀ values. The therapeutic index (TI) is the ratio of IC₅₀ values for vehicle- and etoposide-treated cells. The chemical structure of each compound is shown at the bottom of each plot. ABT-263 had borderline activity at 1 μM and was inactive in the screen at 1 μM (panel (c)), due to marginal decreases in Snc viability. Panel c shows senolytic screening results for 2.352 compounds at a final concentration of 1 μM. Values indicate the mean of two biological replicates, and viability measurements are normalized by the interquartile mean of each cell plate. Two documented senolytics. ABT-737 and A-1331852, were found to be inactive and are highlighted with large points, and two active compounds, sulfisoxazole and imipramine hydrochloride, are highlighted with large points. Sncs were induced with etoposide, and control cells were treated with vehicle (0.5% DMSO). Panel d is similar to (Panel a), but for the screen shown in (Panel c).

FIG. 9 shows comparison of machine learning models. Shown are precision-recall curves for the two-best random forest models, trained and tested on the data shown in Panel 1d of FIG. 1. The dashed curves represent the baseline fraction of active compounds in the training set (1.9%). Light curves and the 95% confidence interval (CI: heavy curves) indicate the variation generated by bootstrapping. AUC, area under the precision-recall curve. The model hyperparameters used were: Panel a: max depth. 5: number of estimators. 80; max features. 20; Panel b: max depth. 5: number of estimators. 40); max features. 40.

FIG. 10 shows chemical filters for favorable medicinal chemistry properties and structural novelty. The numbers of compounds after each chemical filtering step are shown, for both the Broad Institute Drug Repurposing Hub and the extended Broad Institute library. Numbers of curated compounds are indicated at bottom.

FIG. 11 shows screening of 216 compounds with high predicted senolytic activity. 50) compounds with low predicted senolytic activity, and validation of six additional active compounds. Panel a shows relative viability of vehicle- and etoposide-treated cells after a 3-day course of test compound treatment (10 μM). Values are from two biological replicates, and viability measurements are normalized by the interquartile mean of each cell plate. Active compounds are those for which relative control cell viability is >0.5, relative Snc viability is <0.7, and the ratio of Snc to control cell viability is <0.7. All other compounds are inactive. The Pearson's correlation coefficient. R, and two-sided p-value are shown. Panel b shows dose-response curves of control and etoposide-treated IMR-90 cells, treated with each compound shown. Compounds were serially diluted two-fold starting from a final concentration of 50 μM and 0 μM (1% DMSO vehicle) treatment was included. Cells were treated for 3 days. Cellular viability was determined by the metabolic reduction of resazurin into fluorescent resorufin, and values are normalized by the fluorescence intensities of the average of two untreated samples from the same phenotype: here, a cellular viability of 1 indicates that of either untreated control cells or Sncs. Vehicle treatment may result in cellular viability values <1 due to minor effects of DMSO on cellular viability. Curves are shown fo both contorl control (vehicle-treated) cells, and etoposide-treated Sncs. Measurements are shown for two biological replicates in each treatment group (open points), and mean viability values (closed points) were fitted to calculate IC₅₀ values. The therapeutic index (TI) is the ratio of IC₅₀ values for vehicle- and etoposide-treated cells. The chemical structure of each compound is shown at the bottom of each plot.

FIG. 12 shows structural comparisons of identified compounds. Shown are the compounds in the training dataset with highest structural similarity to each of BRD-K20733377. BRD-K56819078, and BRD-K44839765, as measured by the Tanimoto similarity.

FIG. 13 shows BRD-K20733377. BRD-K56819078, and BRD-K44839765 exhibit senolytic activity in a model of doxorubicin-induced senescence. Panel a shows SA-β-gal staining of vehicle-(0.5% DMSO) and doxorubicin-treated IMR-90 cells plated at times on corresponding to the day before and day of compound addition (see also Panel 1b of FIG. 1 and FIG. 7). Each image is representative of two biological replicates. Scale bar. 100 μm. Panel b shows relative mRNA expression of p16, p21, and KI67 in vehicle-(0.5% DMSO), doxorubicin-, and etoposide-treated IMR-90 cells harvested on the day of compound addition. Data for vehicle- and etoposide-treated cells are identical to those shown in Panel c of FIG. 1, and are shown here for comparison. Data from three biological replicates are shown. Error bars indicate one standard deviation. One-way, two-sided ANOVA with Tukey's multiple comparisons: *p≤0.05.** p<0.01.*** p<0.001. Panel c shows dose-response curves of control and doxorubicin-treated IMR-90) cells, treated with each compound shown. Compounds were serially diluted two-fold starting from a final concentration of 50 μM, and 0 μM (1% DMSO vehicle) treatment was included. Cells were treated for 3 days. Cellular viability was determined by the metabolic reduction of resazurin into fluorescent resorufin, and values are normalized by the fluorescence intensities of the average of two untreated samples from the same phenotype: here, a cellular viability of 1 indicates that of either untreated control cells or Sncs. Vehicle treatment may result in cellular viability values <1 due to minor effects of DMSO on cellular viability. Black curves indicate control (vehicle-treated) cells, and light curves indicate (doxorubicin-treated) Sncs. Measurements are shown for two biological replicates in each treatment group (open points), and mean viability values (closed points) were fitted to calculate IC₅₀ values. The therapeutic index (TI) is the ratio of IC₅₀ values for vehicle- and doxorubicin-treated cells. The chemical structure of each compound is displayed in each inset. Data for control cells are identical to those shown in Panels 3a-3d of FIG. 3.

FIG. 14 shows preliminary assessments of compound toxicological properties. Panel a shows fractional hemolysis measurements of human red blood cells treated with BRD-K20733377. BRD-K56819078. BRD-K44839765, and ABT-737 at the indicated final concentrations. Vehicle (1% DMSO) was used as a negative control, and Triton X-100 was used as a positive control. Points indicate values from individual biological replicates, and bars indicate average values. Panel b shows Ames test mutagenesis measurements of the fractions of revertant S, typhimurium TA100 cultures treated with BRD-K20733377. BRD-K56819078. BRD-K44839765, and ABT-737 at a final concentration of 100 μM. Vehicle (1% DMSO) was used as a negative control, and 0.25 μg/mL (˜1 μM) 4-nitroquinoline 1-oxide was used as a positive control. Points indicate values from individual biological replicates, and bars indicate average values. Higher fractions of revertant cultures indicate higher mutagenic potential.

FIG. 15 shows Bcl-XL targeting of compounds disclosed herein. Shown are dose-response curves (black) for each of the indicated compounds. Gray points represent biological replicates, and red points represent mean values.

FIG. 16 shows in vitro testing in senescent human fibroblast cells of compounds disclosed herein. Shown are dose-response curves (black and blue) for each of the indicated compounds. Colors represent control (black) or senescent (blue) IMR-90 fibroblasts, with senescence induced by etoposide treatment.

FIG. 17 shows in vitro testing in senescent human endothelial cells and human primary muscle cells of compounds disclosed herein. Shown are dose-response curves (black and blue) for each of the indicated compounds. Colors represent control (black) or senescent (blue) human primary skeletal muscle cells, with senescence induced by etoposide treatment.

FIGS. 18A-18C shows ex vivo efficacy of IBX-100 for aged skin. FIG. 18A shows a representative image of skin treated with IBX-100, showing no adverse effects or signs of toxicity. Scale bar. 1 mm. FIG. 18B shows data from 3 young samples and 3 old samples. FIG. 18C shows data from 8 vehicle samples and 3 treatment samples. Error bars indicate one standard deviation.

FIGS. 19A-19C shows ex vivo efficacy of IBX-100 for wounded skin. FIG. 19A shows a representative image of skin treated with IBX-100, showing no adverse effects or signs of toxicity. Scale bar. 2 mm. FIG. 19B shows data from 8 wounded samples. FIG. 19C shows from 8 vehicle-treated wounded samples and 8 IBX-100-treated wounded samples. P-values shown in FIG. 19B-19C are for one-sided, two-sample exact permutation tests for differences in mean value compared to respective baselines. Error bars indicate one standard deviation.

FIG. 20 shows dose-response curves for the compounds disclosed herein. Non-cytotoxicity to T-cells is shown for the compounds disclosed.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Cellular senescence is a permanent state of cell cycle arrest that is associated with cellular stress and aging. Although senescence primarily protects against cancer, senescent cells (Sncs) exhibit altered phenotypes and secrete senescence-associated secretory phenotype (SASP) factors, which include cytokines, chemokines, growth factors, and proteases that cause inflammation and tumorigenesis. These factors, in turn, contribute to aging and the deleterious consequences of late-life diseases, including cancer, atherosclerosis and osteoarthritis. Recent studies have shown that the selective clearance of Sncs can ameliorate pathophysiological consequences associated with senescence. In particular, senolytics, an emerging class of drugs that selectively kill Sncs, have been shown to extend healthspan and enhance the efficacy of chemotherapy in mice. Yet, the removal of senescent cells in mice has also been shown to slow wound healing and induce liver and perivascular tissue fibrosis, highlighting the need to discover senolytic therapies that do not induce severe side effects.

Given the potential of senolytics with few side effects to mediate healthy aging, there has been considerable interest in discovering novel senolytics. The first senolytics-dasatinib, quercetin, fisetin, and ABT-263-emerged in the mid-2010s from targeted bioinformatics approaches that focused on pathways protecting Sncs from apoptosis. Subsequently, senolytics including heat shock protein (HSP)-90 inhibitors, cardiac glycosides, and bromodomain and extra-terminal domain (BET) family protein inhibitors have been discovered through high-throughput screens and detailed mechanistic studies. Many of these documented senolytics have side effects or limitations to clinical application. For instance, senolytics including fisetin and ABT-737 have limited bioavailability, and the evaluation of ABT-263 in Phase II studies for the treatment of lung carcinoma revealed that thrombocytopenia and neutropenia were common side effects in patients. Thus, the identification of novel senolytic compounds is needed to advance the development of senolytics as a class of therapeutics.

Parallel to the discovery and development of senolytics, machine learning has proven versatile for facilitating drug discovery efforts. Various machine learning models have combined training data generated from biological screens or available from public databases with architectures including neural networks to predict the activities and pharmacological properties of chemical compounds, discover molecular binding targets and aging biomarkers, and design molecules that satisfy predetermined criteria for biological activity and physicochemical properties. While machine learning approaches have successfully enabled the discovery of chemical compounds targeting diverse indications, including bacterial infection and fibrosis, they remain to be developed, tested, and applied in different therapeutic areas, including senolytics. In such applications, the design of appropriate conceptual frameworks, the generation of well-controlled training data, the choice of suitable model architectures, and the experimental validation of model predictions are important for determining a model's predictive accuracy and demonstrating the utility of machine learning for chemical compound discovery.

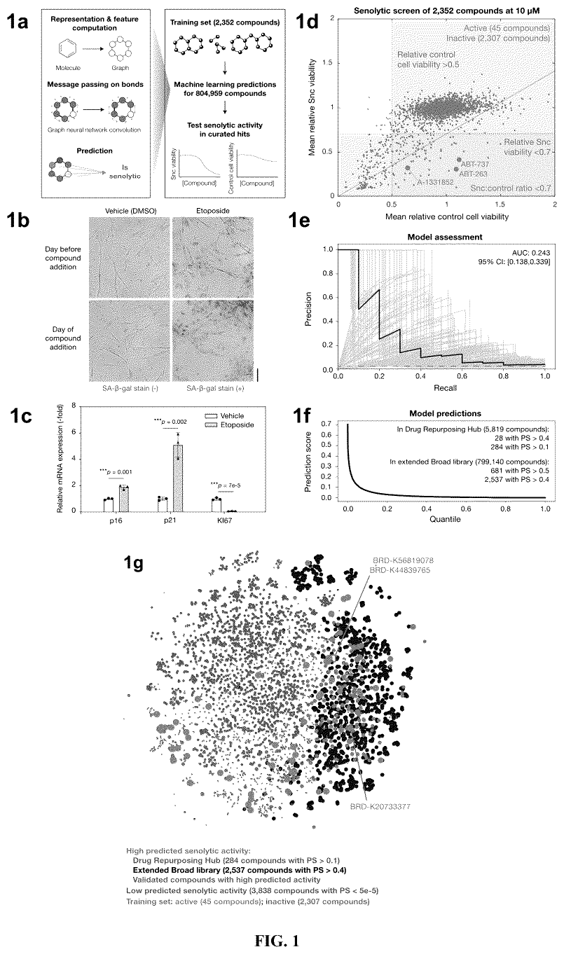

Aspects of the present disclosure provides for in silico prediction of senolytic activity by machine learning models on the basis of chemical structure alone (Panel 1a of FIG. 1). It was found that deep learning models could augment high-throughput screening efforts and identify senolytic compounds from vast chemical spaces. An additional aspect of the present disclosure provides for trained systems and algorithms (e.g., graph neural networks) trained on the results of a screen for senolytic activity of 2.352 compounds and applied it to predict senolytic activity in a chemical space of 804.959 compounds. In contrast to other architectures, graph neural networks enable molecular structures to be directly processed for training and prediction, and this architecture can improve predictive power. In an example, after curating and testing an additional 266 compounds, methods, systems, and algorithms of the disclosure can produce a working hit rate (positive predictive value) of 11.6%, enriching for structurally diverse senolytic compounds with favorable medicinal chemistry properties.

In some embodiments, compounds as disclosed herein are effective senolytics (e.g., IC₅₀<20 μM) and exhibit selectivity comparable to that of ABT-737 in different senescence models.

Methods, systems, and algorithms of the disclosure may comprise molecular docking simulations involving documented senolytic protein targets and/or time-resolved fluorescence energy transfer (TR-FRET) experiments. In some embodiments, compounds of the present disclosure bind Bcl-2. Furthermore, compounds of the present disclosure can have encouraging safety profiles. Compounds of the present disclosure (e.g., BRD-K56819078) can reduce senescent cell burden and senescence-associated mRNA expression in the kidneys of mouse models.

As used in the specification and appended claims, unless specified to the contrary, the following terms have the meaning indicated below.

As used herein. C₁-Cₓ includes C₁-C₂, C₁-C₃ . . . . C₁-Cₓ, C₁-Cₓ refers to the number of carbon atoms that make up the moiety to which it designates (excluding optional substituents).

“Alkyl” or “alkylene” refers to a straight or branched hydrocarbon chain radical consisting solely of carbon and hydrogen atoms, containing no unsaturation, having from one to eighteen carbon atoms (e.g., C₁-C₁₈ alkyl). In certain embodiments, an alkyl comprises three to eighteen carbon atoms (e.g., C₃-C₁₈ alkyl). In certain embodiments, an alkyl comprises one to fifteen carbon atoms (e.g., C₁-C₁₅ alkyl). In certain embodiments, an alkyl comprises one to twelve carbon atoms (e.g., C₁-C₁₂ alkyl). In certain embodiments, an alkyl comprises one to eight carbon atoms (e.g., C₁-C₈ alkyl). In other embodiments, an alkyl comprises one to six carbon atoms (e.g., C₁-C₆ alkyl). In other embodiments, an alkyl comprises one to five carbon atoms (e.g., C₁-C₈alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl). 1.1-dimethylethyl (tert-butyl), and 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{f}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂(where t is 1 or 2) where each R^{a} is independently hydrogen, alkyl, haloalkyl, cycloalkyl, aryl, aralkyl, heterocycloalkyl, heteroaryl, or heteroarylalkyl, and each R^{f} is independently alkyl, haloalkyl, cycloalkyl, aryl, aralkyl, heterocycloalkyl, heteroaryl, or heteroarylalkyl.

“Alkoxy” refers to a radical bonded through an oxygen atom of the formula-O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to eighteen carbon atoms. In certain embodiments, an alkenyl comprises three to eighteen carbon atoms. In certain embodiments, an alkenyl comprises three to twelve carbon atoms. In certain embodiments, an alkenyl comprises six to twelve carbon atoms. In certain embodiments, an alkenyl comprises six to ten carbon atoms. In certain embodiments, an alkenyl comprises eight to ten carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1.4-dienyl, and the like. Unless stated otherwise specifically in the specification, an alkenyl group is optionally substituted by one or more of the following substituents: halo, cyano, nitro, oxo, thioxo, imino, oximo, trimethylsilanyl. —OR^{a}, —SR^{a}, —OC(O)—R^{f}, —N(R^{a})₂, —C(O)R^{a}—C(O)OR^{a}, —C(O)N(R^{a})₂, —N(R^{a})C(O)OR^{f}—OC(O)—NR^{a}R^{f}—N(R^{a})C(O)R^{f}—N(R^{a})S(O)ₜR^{f} (where t is 1 or 2) —S(O)ₜOR^{a} (where t is 1 or 2), —S(O)ₜR^{f} (where t is 1 or 2) and —S(O)ₜN(R^{a}) 2 (where t is 1 or 2) where each R^{a} is independently hydrogen, alkyl, haloalkyl, cycloalkyl, aryl, aralkyl, heterocycloalkyl, heteroaryl, or heteroarylalkyl, and each R^{f} is independently alkyl, haloalkyl, cycloalkyl, aryl, aralkyl, heterocycloalkyl, heteroaryl, or heteroarylalkyl.

“Alkynyl” refers to a straight or branched hydrocarbon chain radical group consisting solely of carbon and hydrogen atoms, containing at least one carbon-carbon triple bond, having from two to eighteen carbon atoms. In certain embodiments, an alkynyl comprises three to eighteen carbon atoms. In certain embodiments, an alkynyl comprises three to twelve carbon atoms. In certain embodiments, an alkynyl comprises six to twelve carbon atoms. In certain embodiments, an alkynyl comprises six to ten carbon atoms. In certain embodiments, an alkynyl comprises eight to ten carbon atoms. In certain embodiments, an alkynyl comprises two to eight carbon atoms. In other embodiments, an alkynyl has two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)ₜR^{f}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, haloalkyl, cycloalkyl, aryl, aralkyl, heterocycloalkyl, heteroaryl, or heteroarylalkyl, and each R^{f} is independently alkyl, haloalkyl, cycloalkyl, aryl, aralkyl, heterocycloalkyl, heteroaryl, or heteroarylalkyl.

“Halo” or “halogen” refers to bromo, chloro, fluoro or iodo substituents.

“Haloalkyl” refers to an alkyl radical, as defined above, that is substituted by one or more halo radicals, as defined above.

“Optional” or “optionally” means that a subsequently described event or circumstance may or may not occur and that the description includes instances when the event or circumstance occurs and instances in which it does not. For example. “optionally substituted aryl” means that the aryl radical are or are not substituted and that the description includes both substituted aryl radicals and aryl radicals having no substitution.

“Pharmaceutically acceptable.” as used herein, refers a material, such as a carrier or diluent, which does not abrogate the biological activity or properties of the compound, and is relatively nontoxic at the concentration or amount used, e.g., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties. Selection and Use. International Union of Pure and Applied Chemistry. Wiley-VCH 2002. S. M. Berge. L. D. Bighley. D. C. Monkhouse. J. Pharm. Sci. 1977, 66, 1-19. P. H. Stahl and C. G. Wermuth, editors. Handbook of Pharmaceutical Salts: Properties. Selection and Use, Weinheim/Zürich: Wiley-VCH/VHCA. 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviors. Also, because the salt-forming molecule can be in equilibrium with a neutral form, passage through biological membranes can be adjusted.

The term “acceptable” with respect to a formulation, composition or ingredient, as used herein, means having no persistent detrimental effect on the general health of the subject being treated.

The terms “administer,” “administering”, “administration,” and the like, as used herein, refer to the methods that may be used to enable delivery of compounds or compositions to the desired site of biological action. These methods include, but not limited to, oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “effective amount” or “therapeutically effective amount,” as used herein, refer to a sufficient amount of an agent or a compound being administered, which will relieve to some extent one or more of the symptoms of the disease or condition being treated. The result includes reduction or alleviation of the signs, symptoms, or causes of a disease, or any other desired alteration of a biological system. For example, an “effective amount” for therapeutic used is the amount of the composition comprising a compound as disclosed herein required to provide a clinically significant decrease in disease symptoms. An appropriate “effective” amount in any individual case is optionally determined using techniques, such as a dose escalation study.

The term “subject” or “patient” encompasses mammals. Examples of mammals include, but are not limited to, any member of the Mammalian class: humans, non-human primates such as chimpanzees, and other apes and monkey species: farm animals such as cattle, horses, sheep, goats, swine: domestic animals such as rabbits, dogs, and cats: laboratory animals including rodents, such as rats, mice and guinea pigs, and the like. In one aspect, the mammal is a human. In some instances, the subject is a tissue or a cell.

As used herein, “treatment” or “treating” or “palliating” or “ameliorating” are used interchangeably herein. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are administered to a patient at risk of developing a particular disease, or to a patient reporting one or more of the physiological symptoms of a disease, even though a diagnosis of this disease has not been made.

### Pharmaceutical Compositions

In some embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Accordingly, provided herein is a pharmaceutical composition comprising at least one compound of Formula (I), or a pharmaceutically acceptable salt thereof, together with one or more pharmaceutically acceptable carriers. In certain embodiments, provided herein are compounds for use in pharmaceutical compositions. In some embodiments, compounds of Formula (I) or pharmaceutically acceptable salts thereof are formulated as formulations with one or more pharmaceutically acceptable excipients.

In certain embodiments, a compound of Formula (I) is represented by:

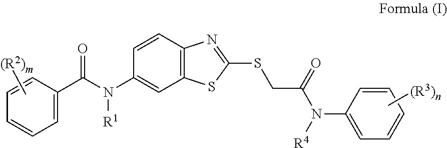

or a pharmaceutically acceptable salt thereof, wherein:
R¹ and R⁴ are each independently hydrogen or C₁₋₆ alkyl;
each R² and R³ is independently selected from the group consisting of halogen, —OR⁵, —SR⁵, —N(R⁵)₂, —C(O)R⁶, —C(O)N(R⁵)₂, —N(R⁵)C(O)R⁶, —C(O)OR⁵, —OC(O)R⁶, —NO₂, —CN, C₁₋₆ alkyl, C₂₋₆ alkenyl, and C₂₋₆ alkynyl: wherein each C₁₋₆ alkyl, C₂₋₆ alkenyl, and C₂₋₆ alkynyl is optionally substituted with one or more substituents independently selected from halogen, —OR⁵, —SR⁵, —N(R⁵)₂, —C(O)R⁶, —C(O)N(R⁵)₂, —N(R⁵)C(O)R⁶, —C(O)OR⁵, —OC(O)R⁶, —NO₂, ═O, and —CN;
each R⁵ is independently selected from: hydrogen; and C₁₋₆ alkyl optionally substituted with one more substituents independently selected from halogen, —O—C₁₋₆ alkyl, —O—C₁₋₆ haloalkyl, —NH₂, —NO₂, ═O, and —CN;
each R⁶ is independently selected from: C₁₋₆ alkyl optionally substituted with one more substituents independently selected from halogen, —O—C₁₋₆ alkyl, —O—C₁₋₆ haloalkyl, —NH₂, —NO₂, ═O, and —CN;
m is 0, 1, 2, 3, 4, or 5; and
n is 0, 1, 2, 3, 4, or 5.

In certain embodiments, a compound of a pharmaceutical formulation described herein is selected from Formula (I):

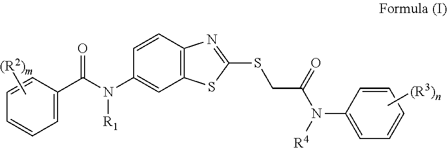

or a pharmaceutically acceptable salt thereof, wherein:
R¹ and R⁴ are each independently hydrogen or C₁₋₆ alkyl;
each R² and R³ is independently selected from the group consisting of halogen, —OR⁵, —SR⁵, —N(R⁵)₂, —C(O)R⁶, —C(O)N(R⁵)₂, —N(R⁵)C(O)R⁶, —C(O)OR⁵, —OC(O)R⁶, —S(O)₂R⁶, —S(O)₂N(R⁵)₂, —NO₂, —CN, C₁₋₆ alkyl, C₂₋₆ alkenyl, and C₂₋₆ alkynyl: wherein each C₁₋₆ alkyl, C₂₋₆ alkenyl, and C₂₋₆ alkynyl is optionally substituted with one or more substituents independently selected from halogen, —OR⁵, —SR⁵, —N(R⁵)₂, —C(O)R⁶, —C(O)N(R⁵)₂, —N(R⁵)C(O)R⁶—C(O)OR⁵, —OC(O)R⁶, —NO₂, ═O, and —CN;
each R⁵ is independently selected from: hydrogen; and C₁₋₆ alkyl optionally substituted with one more substituents independently selected from halogen, —O—C₁₋₆ alkyl, —O—C₁₋₆ haloalkyl, —NH₂, —NO₂, —O, and —CN;
each R⁶ is independently selected from: C₁₋₆ alkyl optionally substituted with one more substituents independently selected from halogen, —O—C₁₋₆ alkyl, —O—C₁₋₆ haloalkyl, —NH₂, —NO₂, ═O, and —CN;
m is 0, 1, 2, 3, 4, or 5; and
n is 0, 1, 2, 3, 4, or 5.

In certain embodiments, for a compound or a pharmaceutical formulation described herein as Formula (I):

R¹ and R⁴ are each independently hydrogen or C₁₋₆ alkyl:
each R² and R³ is independently selected from the group consisting of halogen, —OR⁵, —SR⁵, —N(R⁵)₂, —C(O)R⁶, —C(O)N(R⁵)₂, —N(R⁵)C(O)R⁶, —C(O)OR⁵, —OC(O)R⁶, —NO₂, —CN, C₁₋₆ alkyl, C₂₋₆ alkenyl, and C₂₋₆ alkynyl: wherein each C₁₋₆ alkyl, C₂₋₆ alkenyl, and C₂₋₆ alkynyl is optionally substituted with one or more substituents independently selected from halogen, —OR⁵, —SR⁵, —N(R⁵)₂, —C(O)R⁶, —C(O)N(R⁵)₂, —N(R⁵)C(O)R⁶, —C(O)OR⁵, —OC(O)R⁶, —NO₂, —O, and —CN;
each R⁵ is independently selected from: hydrogen; and C₁₋₆ alkyl optionally substituted with one more substituents independently selected from halogen, —O—C₁₋₆ alkyl, —O—C₁₋₆ haloalkyl, —NH₂, —NO₂, ═O, and —CN;
each R⁶ is independently selected from: C₁₋₆ alkyl optionally substituted with one more substituents independently selected from halogen, —O—C₁₋₆ alkyl, —O—C₁₋₆ haloalkyl, —NH₂, —NO₂, —O, and —CN;
m is 0, 1, 2, 3, 4, or 5; and
n is 0, 1, 2, 3, 4, or 5.

In some embodiments, provided herein is a compound, according to Formula (I), wherein each R² and R³ is independently selected from the group consisting of halogen, —OR⁵, —N(R⁵)₂, and C₁₋₆ alkyl: wherein each C₁₋₆ alkyl, is optionally substituted with one or more substituents independently selected from halogen, —OR⁵, —N(R⁵)₂, —C(O)R⁶, —C(O)N(R⁵)₂, —N(R⁵)C(O)R⁶, —C(O)OR⁵, —OC(O)R⁶, ═O, and —CN: each R⁵ is independently selected from: hydrogen; and C₁₋₆ alkyl optionally substituted with one more substituents independently selected from halogen, —O—C₁₋₆ alkyl, —O—C₁₋₆ haloalkyl, —NH₂. ═O, and —CN; and each R⁶ is independently selected from: C₁₋₆ alkyl optionally substituted with one more substituents independently selected from halogen, —O—C₁₋₆ alkyl, —O—C₁₋₆ haloalkyl, —NH₂, —O, and —CN.

In some embodiments, provided herein is a compound, or a pharmaceutically acceptable salt thereof, according to Formula (I), wherein R⁵ is hydrogen or C₁₋₆ alkyl; and R⁶ is C₁₋₆ alkyl. In some embodiments, provided herein is a compound, or a pharmaceutically acceptable salt thereof, according to Formula (I), wherein R¹ and R⁴ are each hydrogen; and each R² and R³ is independently hydrogen, halogen, —O—C₁₋₆ alkyl, or C₁₋₆ alkyl.

In some embodiments, provided herein is a compound, or a pharmaceutically acceptable salt thereof, according to Formula (I), wherein each R² and R³ is independently-H, —F, —Cl, —OCH₃, —OCH₂CH₃, —CH₃, or —CH₂CH₃. In some embodiments, provided herein is a compound, or a pharmaceutically acceptable salt thereof, according to Formula (I), wherein each R² is independently —Cl, —OCH₃, or —CH₃. In some embodiments, provided herein is a compound, or a pharmaceutically acceptable salt thereof, according to Formula (I), wherein and n is 1, and each R³ is independently —F or —CH₃. In some embodiments, n is 0. In some embodiments, n is 2, and each R³ is —CH₃. In some embodiments, m is 1 and R² is —C₁ or —CH₃. In some embodiments, m is 2 and R² is —OCH₃.

In some embodiments, provided herein is a compound, or a pharmaceutically acceptable salt thereof, according to Formula (I), wherein R² is —OCH₃. In some embodiments, provided herein is a compound, or a pharmaceutically acceptable salt thereof, according to Formula (I), wherein R² is —C₁ or —CH₃. In some embodiments, provided herein is a compound, or a pharmaceutically acceptable salt thereof, according to Formula (I), wherein R³ is —F or —CH₃.

In some embodiments, provided herein is a pharmaceutical composition comprising a compound of Formula (I) represented by one of the following structures:

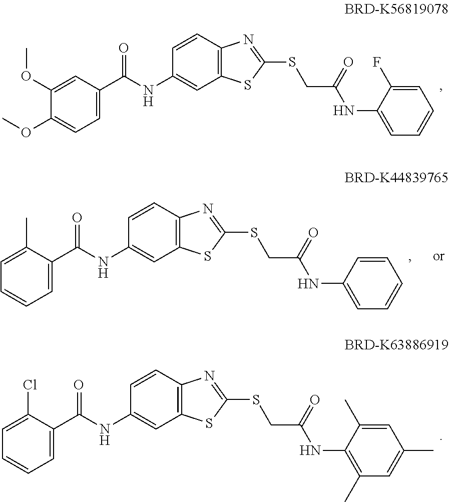 [SMILES: COc1ccc(C(=O)Nc2ccc3nc(SCC(=O)Nc4ccccc4F)sc3c2)cc1OC ; Cc1ccccc1C(=O)Nc1ccc2nc(SCC(=O)Nc3ccccc3)sc2c1 ; Cc1cc(C)c(NC(=O)CSc2nc3ccc(NC(=O)c4ccccc4Cl)cc3s2)c(C)c1]

In some embodiments, provided herein is a pharmaceutical composition comprising the compound:

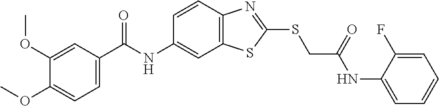 [SMILES: COc1ccc(C(=O)Nc2ccc3nc(SCC(=O)Nc4ccccc4F)sc3c2)cc1OC]

or a pharmaceutically acceptable salt thereof.

In some embodiments, provided herein is a pharmaceutical composition comprising the compound:

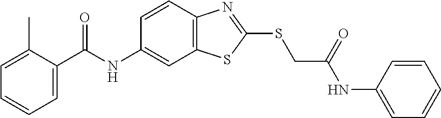 [SMILES: Cc1ccccc1C(=O)Nc1ccc2nc(SCC(=O)Nc3ccccc3)sc2c1]

or a pharmaceutically acceptable salt thereof.

In some embodiments, provided herein is a pharmaceutical composition comprising the compound:

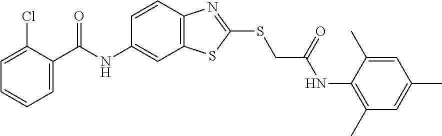 [SMILES: Cc1cc(C)c(NC(=O)CSc2nc3ccc(NC(=O)c4ccccc4Cl)cc3s2)c(C)c1]

or a pharmaceutically acceptable salt thereof.

In another aspect, the present disclosure provides a compound of Formula (II):

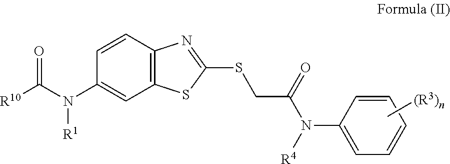

or a pharmaceutically acceptable salt thereof, wherein:
R¹ and R⁴ are each independently hydrogen or C₁₋₆ alkyl;
R¹⁰ is selected from

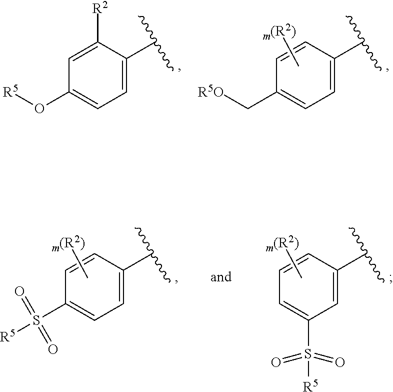

each R² and R³ is independently selected from the group consisting of halogen, —OR⁵, —SR⁵, —N(R⁵)₂, —C(O)R⁶, —C(O)N(R⁵)₂, —N(R⁵)C(O)R⁶, —C(O)OR⁵, —C(O)OR⁵, —OC(O)R⁶, —S(O)₂R⁶, —S(O)₂N(R⁵)₂, —NO₂, —CN, C₁₋₆ alkyl, C₂₋₆ alkenyl, and C₂₋₆ alkynyl; wherein each C₁₋₆ alkyl, C₂₋₆ alkenyl, and C₂₋₆ alkynyl is optionally substituted with one or more substituents independently selected from halogen, —OR⁵, —SR⁵, —N(R⁵)₂, —C(O)R⁶, —C(O)N(R⁵)₂, —N(R⁵)C(O)R⁶, —C(O)OR⁵, —OC(O)R⁶, —NO₂, ═O, and —CN;
each R⁵ is independently selected from: hydrogen; and C₁₋₆ alkyl optionally substituted with one more substituents independently selected from halogen, —O—C₁₋₆ alkyl, —O—C₁₋₆ haloalkyl, —NH₂, —NO₂, —O, and —CN;
each R⁶ is independently selected from: C₁₋₆ alkyl optionally substituted with one more substituents independently selected from halogen, —O—C₁₋₆ alkyl, —O—C₁₋₆ haloalkyl, —NH₂, —NO₂, ═O, and —CN;
m is 0, 1, 2, 3, or 4; and
n is 0, 1, 2, 3, 4, or 5.

In some embodiments of a compound of Formula (II),
R¹ and R⁴ are each hydrogen;
m is selected from 0 and 1;
R¹⁰ is selected from

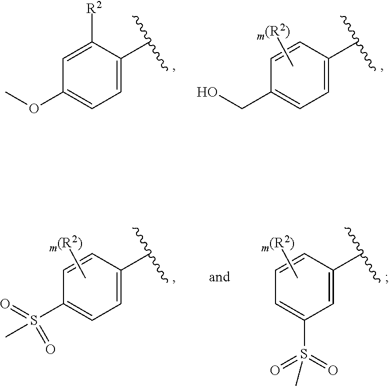

n is selected from 0 and 1; and
each R³ is independently halogen, and —S(O)₂N(R⁵)₂.

In some embodiments of a compound or salt of Formula (II), each m is selected from 0 and 1, each R² is independently selected from the group consisting of halogen, n is selected from 0 and 1; and each R³ is independently halogen, and —S(O)₂NH₂. In some cases, m is 0. In some cases, m is 1. In some cases, R² is fluorine. In some cases, n is 0. In some cases, n is 1. In some cases, R³ is —S(O)₂NH₂. In some cases, R³ is halogen. In some cases, R³ is selected from chlorine and fluorine. In some cases, R³ is Cl. In some cases, R³ is F.

In some embodiments of a compound or salt of Formula (II), each R⁵ is independently selected from: hydrogen; and C₁₋₆ alkyl optionally substituted with one more substituents independently selected from halogen, —O—C₁₋₆ alkyl, —O—C₁₋₆ haloalkyl, —NH₂, and —CN. In some cases, each R⁵ is independently selected from: hydrogen; and C₁₋₆ alkyl. In some cases, each R⁵ is independently selected from: hydrogen; and methyl. In some cases, R⁵ is methyl. In some cases, R⁵ is hydrogen.

In some embodiments of a compound or salt of Formula (II), R¹⁰ is selected from

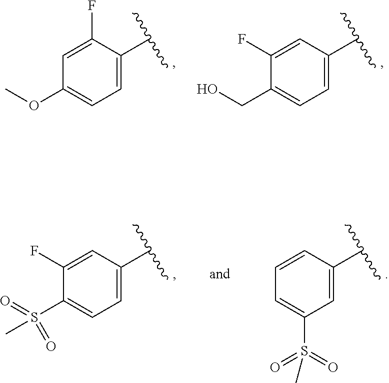

In some cases, R¹⁰ is selected from

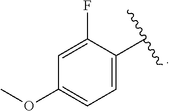 [SMILES: *c1ccc(OC)cc1F]

In some cases, R¹⁰ is

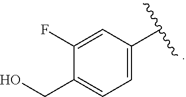 [SMILES: *c1ccc(CO)c(F)c1]

In some cases, R¹⁰ is

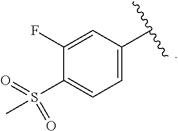 [SMILES: *c1ccc(S(C)(=O)=O)c(F)c1]

In some cases, R¹⁰ is

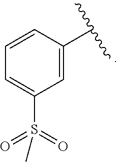 [SMILES: *c1cccc(S(C)(=O)=O)c1]

In another aspect, the present disclosure provides a pharmaceutical composition comprising a compound of Formula (II) and a pharmaceutically acceptable excipient.

The carrier(s) (or excipient(s)) is acceptable or suitable if the carrier is compatible with the other ingredients of the composition and not deleterious to the recipient (i.e., the subject) of the composition.

Exemplary pharmaceutical compositions are used in the form of a pharmaceutical preparation, for example, in solid, semisolid or liquid form, which includes one or more of a disclosed compound, as an active ingredient, in a mixture with an organic or inorganic carrier or excipient suitable for external, enteral or parenteral applications. In some embodiments, the active ingredient is compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

In some embodiments for preparing solid compositions such as tablets, the principal active ingredient is mixed with a pharmaceutical carrier, e.g., conventional tableting ingredients such as corn starch, lactose, sucrose, sorbitol, talc, stearic acid, magnesium stearate, dicalcium phosphate, or gums, and other pharmaceutical diluents, e.g., water, to form a solid preformulation composition containing a homogeneous mixture of a disclosed compound or a non-toxic pharmaceutically acceptable salt thereof. When referring to these preformulation compositions as homogeneous, it is meant that the active ingredient is dispersed evenly throughout the composition so that the composition is readily subdivided into equally effective unit dosage forms such as tablets, pills and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, cellulose, microcrystalline cellulose, silicified microcrystalline cellulose, lactose, sucrose, glucose, mannitol, and/or silicic acid: (2) binders, such as, for example, carboxymethylcellulose, hypromellose, alginates, gelatin, polyvinyl pyrrolidone, sucrose and/or acacia: (3) humectants, such as glycerol: (4) disintegrating agents, such as crospovidone, croscarmellose sodium, sodium starch glycolate, agar-agar, calcium carbonate, potato or tapioca starch, alginic acid, certain silicates, and sodium carbonate: (5) solution retarding agents, such as paraffin: (6) absorption accelerators, such as quaternary ammonium compounds: (7) wetting agents, such as, for example, docusate sodium, cetyl alcohol and glycerol monostearate: (8) absorbents, such as kaolin and bentonite clay: (9) lubricants, such a talc, calcium stearate, magnesium stearate, solid polyethylene glycols, sodium lauryl sulfate, and mixtures thereof; and (10) coloring agents. In the case of capsules, tablets and pills, in some embodiments, the compositions comprise buffering agents. In some embodiments, solid compositions of a similar type are also employed as fillers in soft and hard-filled gelatin capsules using such excipients as lactose or milk sugars, as well as high molecular weight polyethylene glycols and the like.

In some embodiments, a tablet is made by compression or molding, optionally with one or more accessory ingredients. In some embodiments, compressed tablets are prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. In some embodiments, molded tablets are made by molding in a suitable machine a mixture of the subject composition moistened with an inert liquid diluent. In some embodiments, tablets, and other solid dosage forms, such as dragees, capsules, pills, and granules, are scored or prepared with coatings and shells, such as enteric coatings and other coatings.

### Methods of Treatment

In some embodiments, compounds of Formula (I) or (II) may be used in a method of treating a disease or disorder, for example, a disease or disorder associated with aging. Accordingly, provided herein is a method of treating a disease or disorder in a subject, the method comprising administering to the subject a therapeutically effective amount of a compound according to Formula (I), or a pharmaceutically acceptable salt thereof. Also provided herein is a method of treating a disease or disorder in a subject, the method comprising administering to the subject a pharmaceutical composition comprising a compound of Formula (I) or (II),

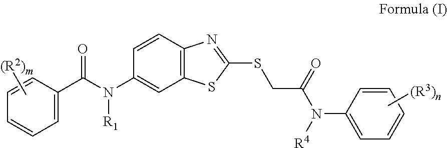

or a pharmaceutically acceptable salt thereof and a pharmaceutically acceptable excipient, wherein:
R¹ and R⁴ are each independently hydrogen or C₁₋₆ alkyl;
each R² and R³ is independently selected from the group consisting of halogen, —OR⁵, —SR⁵, —N(R⁵)₂, —C(O)R⁶, —C(O)N(R⁵)₂, —N(R⁵)C(O)R⁶, —C(O)OR⁵, —OC(O)R⁶, —NO₂, —CN, C₁₋₆ alkyl, C₂₋₆ alkenyl, and C₂₋₆ alkynyl; wherein each C₁₋₆ alkyl, C₂₋₆ alkenyl, and C₂₋₆ alkynyl is optionally substituted with one or more substituents independently selected from halogen, —OR⁵, —SR⁵, —N(R⁵)₂, —C(O)R⁶, —C(O)N(R⁵)₂, —N(R⁵)C(O)R⁶, —C(O)OR⁵, —OC(O)R⁶, —NO₂, ═O, and —CN;
each R⁵ is independently selected from: hydrogen; and C₁₋₆ alkyl optionally substituted with one more substituents independently selected from halogen, —O—C₁₋₆ alkyl, —O—C₁₋₆ haloalkyl, —NH₂, —NO₂, —O, and —CN;
each R⁶ is independently selected from: C₁₋₆ alkyl optionally substituted with one more substituents independently selected from halogen, —O—C₁₋₆ alkyl, —O—C₁₋₆ haloalkyl, —NH₂, —NO₂, ═O, and —CN;
m is 0, 1, 2, 3, 4, or 5; and
n is 0, 1, 2, 3, 4, or 5.

In some embodiments, provided herein is a method of treating a disease or disorder in a subject, the method comprising administering to the subject a pharmaceutical composition comprising a compound represented by one of the following structures:

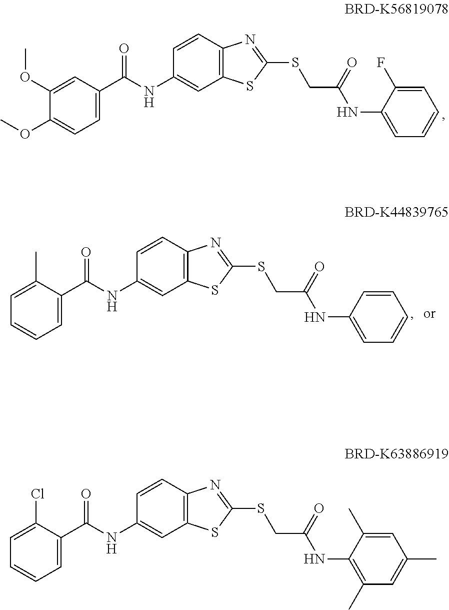 [SMILES: COc1ccc(C(=O)Nc2ccc3nc(SCC(=O)Nc4ccccc4F)sc3c2)cc1OC ; Cc1ccccc1C(=O)Nc1ccc2nc(SCC(=O)Nc3ccccc3)sc2c1 ; Cc1cc(C)c(NC(=O)CSc2nc3ccc(NC(=O)c4ccccc4Cl)cc3s2)c(C)c1]

or a pharmaceutically acceptable salt of any one thereof, and a pharmaceutically acceptable excipient.

In some embodiments, provided herein is a method of treating a disease or disorder in a subject, the method comprising administering to the subject a therapeutically effective amount of the compound:

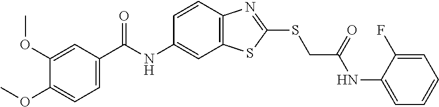 [SMILES: COc1ccc(C(=O)Nc2ccc3nc(SCC(=O)Nc4ccccc4F)sc3c2)cc1OC]

or a pharmaceutically acceptable salt thereof.

In some embodiments, the compound is formulated in a pharmaceutical composition further comprising one or more pharmaceutically acceptable excipients prior to the administering to the subject.

In some embodiments, the disease or disorder is a senescence-associated disease or disorder. In some embodiments, the senescence-associated disease or disorder is an age-related disease or disorder. In some embodiments, the disease or disorder is a senescence-associated disease or disorder, or an age-related disease or disorder described herein.

In some embodiments, provided herein is a method of decreasing senescent cell burden and/or mRNA expression of senescence-associated genes in a subject (e.g., a tissue or a cell) comprising contacting the senescent cell with a senolytic agent described herein. Also provided herein are methods for selectively killing a senescent cell comprising contacting the senescent cell with a senolytic agent described herein (i.e., facilitating interaction or in some manner allowing the senescent cell and senolytic agent to interact) under conditions and for a time sufficient to kill the senescent cell. In such embodiments, the agent selectively kills senescent cells over non-senescent cells (i.e., the agent selectively kills senescent cells compared with killing of non-senescent cells). In certain embodiments, the senescent cell to be killed is present in a subject (e.g., a human or non-human animal). The senolytic agent(s) may be administered to the subject according to the treatment cycles, treatment courses, and non-treatment intervals described above and herein.

In some embodiments, the compounds of Formula (I) or (II) may be applicable for diverse indications where senescent cell accumulation is a driver of disease pathology. In some embodiments, the compounds of Formula (I) or (II) are senescent cell-targeting, have favorable medicinal chemistry properties, and lack appreciable toxicity. In some embodiments, the compounds of Formula (I) or (II) may be relevant to medical aesthetics comprising decreasing biological age of skin, fibrosis and scarring, inflammation and inflammaging, sunburn and skin damage, and/or treatment of DNA-damanged skin.

In some embodiments, the compounds of Formula (I) or (II) may decrease the mRNA expression of p16, a key age-associated biomarker, as well as Col1a1 and MMP9 in aged or wounded ex vivo human skin models suggests that the compounds may improve tissue function and wound healing by eliminating senescent cells and reducing scarring. In some embodiments, the compounds of Formula (I) or (II) may decrease the mRNA expression of Col1a1. In some embodiments, the compounds provided herein may decrease the mRNA expression of MMP9.

### Pharmaceutical Formulation

In some embodiments, a compound of Formula (I), a compound of Formula (II), or a pharmaceutical composition of any one thereof, is formulated into a pharmaceutical formulation In some cases, the pharmaceutical formulation is a 5% topical ointment in 10% DMSO, 45% PEG300, and 45% water. In some cases, the formulation contains at least 5%, 6%, 7%, 8%, 9%, 10%, 11%, 12%, 13%, 14%, or 15% DMSO. In some cases, the formulation contains at most 15%, 14%, 13%, 12%, 11%, 10%, 9%, 8%, 7%, 6%, or 5% DMSO. In some cases, the formulation contains at least 40%, 41%, 42%, 43%, 44%, 45%, 46%, 47%, 48%, 49%, or 50% PEG300. In some cases, the formulation contains at most 50%, 49%, 48%, 47%, 46%, 45%, 44%, 43%, 42%, 41%, or 40% PEG300. In some cases, the formulation contains at least 40%, 41%, 42%, 43%, 44%, 45%, 46%, 47%, 48%, 49%, or 50% water. In some cases, the formulation contains at most 50%, 49%, 48%, 47%, 46%, 45%, 44%, 43%, 42%, 41%, or 40% water.

### Senescence-Associated Diseases and Disorders

Methods are provided herein for treating conditions, diseases, or disorders related to, associated with, or caused by cellular senescence, including age-related diseases and disorders in a subject in need thereof. Methods are provided herein for treating conditions, diseases, or disorders related to, associated with, or caused by cellular senescence, including age-related diseases and disorders in a subject in need thereof by administering to the subject in need thereof a compound of Formula (I).

A senescence-associated disease or disorder may also be called herein a senescent cell-associated disease or disorder. Senescence-associated diseases and disorders include, for example, cardiovascular diseases and disorders, inflammatory diseases and disorders, autoimmune diseases and disorders, pulmonary diseases and disorders, eve diseases and disorders, metabolic diseases and disorders, neurological diseases and disorders (e g., neurodegenerative diseases and disorders); age-related diseases and disorders induced by senescence: skin conditions; age-related diseases; dermatological diseases and disorders; and transplant related diseases and disorders. A prominent feature of aging is a gradual loss of function, or degeneration that occurs at the molecular, cellular, tissue, and organismal levels. Age-related degeneration gives rise to well-recognized pathologies, such as sarcopenia, atherosclerosis and heart failure, osteoporosis, pulmonary insufficiency, renal failure, neurodegeneration (including macular degeneration, Alzheimer's disease, and Parkinson's disease), and many others. Although different mammalian species vary in their susceptibilities to specific age-related pathologies, collectively, age-related pathologies generally rise with approximately exponential kinetics beginning at about the mid-point of the species-specific life span (e.g., 50-60) years of age for humans).

Examples of senescence-associated conditions, disorders, or diseases that may be treated by administering any one of the senolytic agents (e.g., compounds of Formula (I)) described herein according to the methods described herein include, cognitive diseases (e g., mild cognitive impairment (MCI). Alzheimer's disease and other dementias: Huntington's disease); cardiovascular disease (e.g., atherosclerosis, cardiac diastolic dysfunction, aortic aneurysm, angina, arrhythmia, cardiomyopathy, congestive heart failure, coronary artery disease, myocardial infarction, endocarditis, hypertension, carotid artery disease, peripheral vascular diseases, cardiac stress resistance, cardiac fibrosis): metabolic diseases and disorders (e.g., obesity, diabetes, metabolic syndrome): motor function diseases and disorders (e.g., Parkinson's disease, motor neuron dysfunction (MND), Huntington's disease): cerebrovascular disease: emphysema; osteoarthritis: benign prostatic hypertrophy: pulmonary diseases (e.g., idiopathic pulmonary fibrosis, chronic obstructive pulmonary disease (COPD), emphysema obstructive bronchiolitis, asthma): inflammatory/autoimmune diseases and disorders (e.g., osteoarthritis, eczema, psoriasis, osteoporosis, mucositis, transplantation related diseases and disorders), ophthalmic diseases or disorders (e.g., age-related macular degeneration, cataracts, glaucoma, vision loss, presbyopia); diabetic ulcer; metastasis: a chemotherapeutic side effect, a radiotherapy side effect; aging-related diseases and disorders (e.g., kyphosis, renal dysfunction, frailty, hair loss, hearing loss, muscle fatigue, skin conditions, sarcopenia, and herniated intervertebral disc) and other age-related diseases that are induced by senescence (e.g., diseases/disorders resulting from irradiation, chemotherapy, smoking tobacco, eating a high fat/high sugar diet, and environmental factors), wound healing: skin nevi: fibrotic diseases and disorders (e g. , cystic fibrosis, renal fibrosis, liver fibrosis, pulmonary fibrosis, oral submucous fibrosis, cardiac fibrosis, and pancreatic fibrosis). In certain embodiments, any one or more of the diseases or disorders described above or herein may be excluded.

In a more specific embodiment, methods are provided for treating a senescence-associated disease or disorder by killing senescent cells (i.e., established senescent cells) associated with the disease or disorder in a subject who has the disease or disorder by administering a senolytic agent, where m the disease or disorder is osteoarthritis: idiopathic pulmonary fibrosis: chronic obstructive pulmonary disease (COPD); or atherosclerosis.

Subjects (i.e., patients, individuals (human or non-human animals)) who may benefit from use of the methods described herein that comprise administering a senolytic agent (e.g., a compound of Formula (I)) include those who may also have a cancer. The subject treated by these methods may be considered to be in partial or complete remission (also called cancer remission) As discussed in detail herein, the senolytic agents for use in methods for selective killing of senescent cells are not intended to be used as a treatment for cancer, that is, in a manner that kills or destroys the cancer cells in a statistically significant manner. Therefore, the methods disclosed herein do not encompass use of the senolytic agents m a manner that would be considered a primary therapy for the treatment of a cancer. Even though a senolytic agent, alone or with other chemotherapeutic or radiotherapy agents, are not used in a manner that is sufficient to be considered as a primary cancer therapy, the methods and senolytic agents described herein may be used in a manner (e.g, a short term course of therapy) that is useful for inhibiting metastases. In other certain embodiments, the subject to be treated with the senolytic agent does not have a cancer (i.e., the subject has not been diagnosed as having a cancer by a person skilled in the medical art).

### Age-Related Diseases and Disorders

A senolytic agent may also be useful for treating or preventing (I.e., reducing the likelihood of occurrence) of an age-related disease or disorder that occurs as part of the natural aging process or that occurs when the subject is exposed to a senescence inducing agent or factor (e.g., irradiation, chemotherapy, smoking tobacco, high-fat/high sugar diet, other environmental factors). An age-related disorder or disease or an age-sensitive trait may be associated with a senescence-inducing stimulus. The efficacy of a method of treatment described herein may be manifested by reducing the number of symptoms of an age-related disorder or age-sensitive trait associated with a senescence-inducing stimulus, decreasing the severity of one or more symptoms, or delaying the progression of an age-related disorder or age-sensitive trait associated with a senescence-inducing stimulus. In other particular embodiments, preventing an age-related disorder or age-sensitive trait associated with a senescence-inducing stimulus refers to preventing (i.e., reducing the likelihood of occurrence) or delaying onset of an age-related disorder or age-sensitive trait associated with a senescence-inducing stimulus, or reoccurrence of one or more age-related disorder or age-sensitive trait associated with a senescence-inducing stimulus. Age related diseases or conditions include, for example, renal dysfunction, kyphosis, herniated intervertebral disc, frailty, hair loss, hearing loss, vision loss (blindness or impaired vision), muscle fatigue, skin conditions, skin nevi, diabetes, metabolic syndrome, and sarcopenia. Vision loss refers to the absence of vision when a subject previously had vision Various scales have been developed to describe the extent of vision and vision loss based on visual acuity. Age-related diseases and conditions also include dermatological conditions, for example without limitation, treating one or more of the following conditions: wrinkles, including superficial fine wrinkles; hyperpigmentation; scars; keloid; dermatitis; psoriasis; eczema (including seborrheic eczema); rosacea; vitiligo; ichthyosis vulgaris: dermatomyositis; and actinic keratosis.

Frailty has been defined as a clinically recognizable state of increased vulnerability resulting from aging-associated decline in reserve and function across multiple physiologic systems that compromise a subject's ability to cope with every day or acute stressors. Frailty has been may be characterized by compromised energetics characteristics such as low grip strength, low energy, slowed waking speed, low physical activity, and/or unintentional weight loss. Studies have suggested that a patient may be diagnosed with frailty when three of five of the foregoing characteristics are observed. In certain embodiments, aging and diseases and disorders related to aging may be treated or prevented (i.e., the likelihood of occurrence of is reduced) by administering a senolytic agent. The senolytic agent may inhibit senescence of adult stem cells or inhibit accumulation, kill, or facilitate removal of adult stem cells that have become senescent.

The effectiveness of a senolytic agent with respect to treating a senescence-associated disease or disorder described herein can readily be determined by a person skilled in the medical and clinical arts. One or any combination of diagnostic methods appropriate for the particular disease or disorder, which methods are well known to a person skilled in the art, including physical examination, patient self-assessment, assessment and monitoring of clinical symptoms, performance of analytical tests and methods, including clinical laboratory tests, physical tests, and exploratory surgery, for example, may be used for monitoring the health status of the subject and the effectiveness of the senolytic agent. The effects of the methods of treatment described herein can be analyzed using techniques known in the art, such as comparing symptoms of patients suffering from or at risk of a particular disease or disorder that have received the pharmaceutical composition comprising a senolytic agent with those of patients who were not treated with the senolytic agent or who received a placebo treatment.

As understood by a person skilled in the medical art, the terms. “treat” and “treatment.” refer to medical management of a disease, disorder, or condition of a subject (i.e., patient). In general, an appropriate dose and treatment regimen provide the senolytic agent in an amount sufficient to provide therapeutic and/or prophylactic benefit Therapeutic benefit for subjects to whom the senolytic agents described herein are administered, includes, for example, an improved clinical outcome, wherein the object is to prevent or slow or retard (lessen) an undesired physiological change associated with the disease, or to prevent or slow or retard (lessen) the expansion or severity of such disease. As discussed herein, effectiveness of the one or more senolytic agents may include beneficial or desired clinical results that comprise, but are not limited to, abatement, lessening, or alleviation of symptoms that result from or are associated with the disease to be treated; decreased occurrence of symptoms: improved quality of life; longer disease-free status (i.e., decreasing the likelihood or the propensity that a subject will present symptoms on the basis of which a diagnosis of a disease is made): diminishment of extent of disease, stabilized (i.e., not worsening) state of disease; delay or slowing of disease progression; amelioration or palliation of the disease state; and remission (whether partial or total), whether detectable or undetectable; and/or overall survival. The effectiveness of the senolytic agents described herein may also mean prolonging survival when compared to expected survival if a subject were not receiving the senolytic agent that selectively kills senescent cells.

Administration of a senolytic agent described herein can prolong prolonging survival when compared to expected survival if a subject were not receiving treatment. Subjects in need of treatment include those who already have the disease or disorder as well as subjects prone to have or at risk of developing the disease or disorder, and those in which the disease, condition, or disorder is to be treated prophylactically. A subject may have a genetic predisposition for developing a disease or disorder that would benefit from clearance of senescent cells or may be of a certain age wherein receiving a senolytic agent would provide clinical benefit to delay development or reduce severity of a disease, including an age-related disease or disorder.

In another embodiment, a method is provided for treating a senescence-associated disease or disorder that further comprises identifying a subject who would benefit from treatment with a senolytic agent described herein (i.e., phenotyping: individualized treatment). This method comprises first detecting the level of senescent cells in the subject, such as in a particular organ or tissue of the subject A biological sample may be obtained from the subject, for example, a blood sample, serum or plasma sample, biopsy specimen, body fluids (e.g., lung lavage, ascites, mucosal washings, synovial fluid, vitreous fluid, spinal fluid), bone marrow, lymph nodes, tissue explant, organ culture, or any other tissue or cell preparation from a subject. The level of senescent cells may be determined according to any of the in vitro assays or techniques described herein. For example, senescence cells may be detected by morphology (as viewed by microscopy, for example), production of senescence associated markers such as, senescence-associated β-galactosidase (SA-β-gal), p16INK4a, p21. PAI-1, or any one or more SASP factors (e.g., IL-6. MMP3). The senescent cells and non-senescent cells of the biological sample may also be used in an in vitro cell assay in which the cells are exposed to any one of the senolytic agents described herein to determine the capability of the senolytic agent to kill the subject's senescent cells without undesired toxicity to non-senescent cells. As positive controls in these assays, the assay may incorporate any one of the senolytic agents described herein. The subject then may be treated with an appropriate senolytic agent, which may be a MDM2 mhibitor; an inhibitor of one or more Bel-2 anti-apoptotic protein family members wherein the inhibitor inhibits at least Bel-XL (e.g., a Bcl-xL selective inhibitor, Bel-2/Bel-xL/Bel-w inhibitor, a Bcl-2/Bel-xL or a Bel-xL/Bcl-w inhibitor); or an Akt specific inhibitor. In addition, these methods may be used to monitor the level of senescent cells in the subject before, during, and after treatment with a senolytic agent. In certain embodiments, the presence of senescence cells, may be detected (e.g., by determining the level of a senescent cell marker expression of mRNA, for example), and the treatment course and/or non-treatment interval can be adjusted accordingly.

A subject, patient, or individual in need of treatment with a senolytic agent as described herein may be a human or may be a non-human primate or other animal (i.e., veterinary use) who has developed symptoms of a senescence cell-associated disease or disorder or who is at risk for developing a senescence cell-associated disease or disorder. Non-human animals that may be treated include mammals, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, elephants, bears and other domestic, farm, and zoo animals.

### EXAMPLES

### Example 1—Identification of Senolytics with Deep Neural Networks

Cell culture. Human lung fibroblast (IMR-90) cells were obtained from ATCC (CCL-186) and passaged less than 10 times (less than 30 population doublings) for all experiments described in this example, with the exception of high-passage cells used in the model of replicative senescence (see the section on Replicative senescence below). Cells were cultured in growth media comprising Eagle's Minimum Essential Medium (EMEM: ATCC 30-2003) supplemented with 10% fetal bovine serum (FBS: Thermo Fisher 16140071) and 1% penicillin-streptomycin (Thermo Fisher 15070063). Cells were incubated in a humidity-controlled incubator at 37° C., with 5% CO₂. Cells were detached using trypsin-EDTA (0.05%; Gibco 25300120).

Etoposide-induced senescence. IMR-90 cells were cultured as described above. At between 30 to 50% confluence, the media was replaced with complete growth media containing vehicle (0.5% dimethyl sulfoxide: DMSO, MilliporeSigma D5879) or complete growth media containing 50 μM etoposide (prepared as a 1:200 dilution of a 10 mM stock solution in DMSO; etoposide, MilliporeSigma E1383). Cells were treated for 2 days, after which the media was replaced with fresh growth media and cells were allowed to recover for 4 days. Cells were then plated as described below.

Doxorubicin-induced senescence. IMR-90 cells were cultured as described above. At between 30 to 50% confluence, the media was replaced with complete growth media containing vehicle (0.5% DMSO) or complete growth media containing 0.5 μM doxorubicin (prepared as a 1:200 dilution of a 0.1 mM stock solution in DMSO: doxorubicin, Cayman Chemical 15007). Cells were treated for 2 days, after which the media was replaced with fresh growth media and cells were allowed to recover for 4 days. Cells were then plated as described below.

Replicative senescence. Early-passage IMR-90 cells (passage number <3, corresponding to less than 9 population doublings from supplier's stock) were used. High-passage IMR-90 cells were cultured as described above and passaged until cells became non-dividing (passage number >10, corresponding to at least 30 population doublings from supplier's stock). Senescence was confirmed with SA-β-gal staining and mRNA quantification, as described elsewhere herein. Cells were then plated as described below.

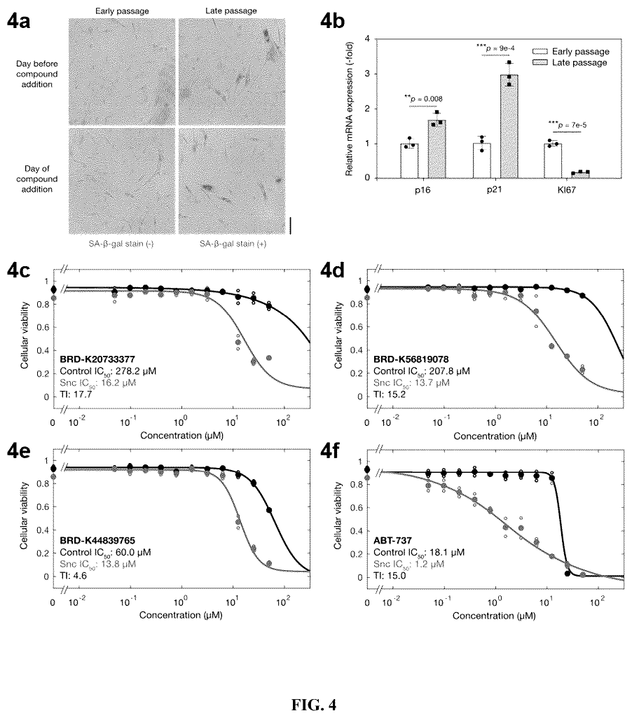

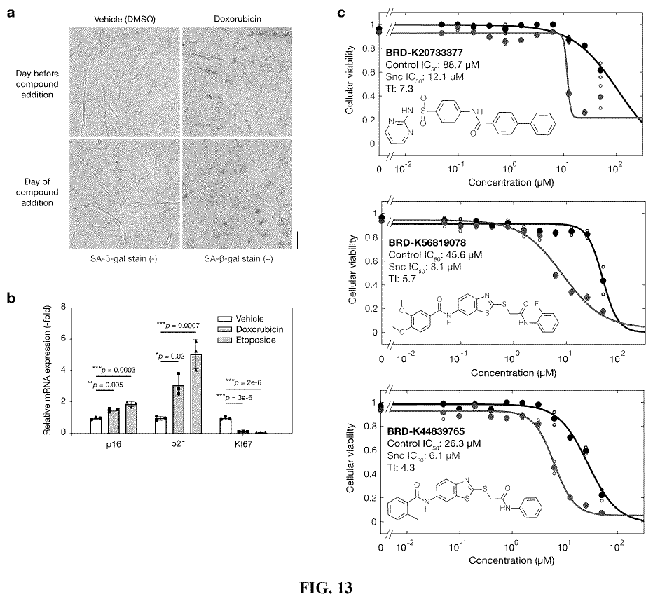

Senescence-associated β-galactosidase staining. On each of two days before or on the day of compound addition—corresponding to days 0 and 1 shown in FIG. 7—cells treated with vehicle, etoposide, or doxorubicin (models of therapy-induced senescence) or early-passage and late-passage cells (model of replicative senescence) were plated onto 6-well plates at an initial density of 0.2 to 0.5×10⁶ cells/well. Cells were then incubated overnight for adhesion. The following day, SA-β-gal staining was performed using a commercial staining kit (Cell Signaling Technology 9860) following the manufacturer's instructions. Briefly, cells in each well were rinsed once with 2 mL of Dulbecco's phosphate-buffered saline (DPBS: VWR 02-0119-0500). Cells in each well were fixed for 15 min at room temperature using 1 mL of 1× fixative solution. Next, cells in each well were rinsed twice with DPBS, then 1 mL of β-galactosidase staining solution (pH 6.0) was added. Plates were sealed with parafilm and incubated overnight at 37° C., in a dry incubator. The next day, cells in each well were imaged with a light microscope to detect staining, as shown in Panel 1b of FIG. 1, Panel 4a of FIG. 4, and FIG. 13.

mRNA quantification using quantitative PCR. Total RNA was extracted using an RNeasy& mini kit from Qiagen (Qiagen 74104) following the manufacturer's instructions. For qPCR analysis, cDNAs were synthesized using a QuantiTect® Reverse Transcription Kit from Qiagen following the manufacturer's instructions (Qiagen 205311). Real-time PCR amplifications were performed in 96-well optical reaction plates using a Power SYBR™ Green PCR Master Mix from Thermo-Fisher (Thermo-Fisher 4368577). The following primers were used, and the relative expression of each gene was determined by normalization to GAPDH expression for each sample:

p16 Forward primer:
CCCAACGCACCGAATAGTTA
p16 Reverse primer:
ACCAGCGTGTCCAGGAAG
p21 Forward primer:
TGTCCGTCAGAACCCATGC
p21 Reverse primer:
AAAGTCGAAGTTCCATCGCTC
KI67 Forward primer:
GAGGTGTGCAGAAAATCCAAA
KI67 Reverse primer:
CTGTCCCTATGACTTCTGGTTGT
GAPDH Forward primer:
GGAGCGAGATCCCTCCAAAAT
GAPDH Reverse primer:
GGCTGTTGTCATACTTCTCATGG

Relative expression values for each of p16, p21, and KI67 were then normalized to those of control (DMSO-treated or early passage) cells for comparison, as shown in Panel 1c of FIG. 1. Panel 4b of FIG. 4, and FIG. 13.

Chemical compounds for screens. A screening library was obtained from MicroSource Discovery Systems comprising FDA-approved drugs, drugs currently in clinical trials, and natural products (2.560) compounds total). The screening library was supplemented with 20 compounds, most of which have documented senolytic activity, and all of which were procured from commercial suppliers. After deduplication of 228 compounds, the 2.352 unique compounds were screened for senolytic activity as described below.

Chemical screening. For all screening experiments. 99 μL of cells were plated into each well of a 96-well clear flat-bottom, black polystyrene tissue-culture-treated plate (Corning 3904) at a density of ˜10+ cells/well. Plates were incubated overnight for adhesion. The day after plating. 1 μL of each compound, prepared as either a 1 mM stock solution in DMSO (10 μM final concentration screen) or 0.1 mM stock solution in DMSO (1 μM final concentration screen), was added to each well using an Agilent Bravo liquid handler. A built-in slow mixing step involving aspirating and dispensing was used to enhance distribution of the added compounds in solution. Cells were incubated for 3 days, after which resazurin (MilliporeSigma R⁷⁰¹⁷) was added to each well to a final concentration of 0.15 mM. After an additional 1 day of incubation, the fluorescence excitation/emission at 550/590 nm was read using a SpectraMaxR M3 plate reader and manufacturer software (SoftMax Pro R 6). Experiments were performed in biological duplicate.

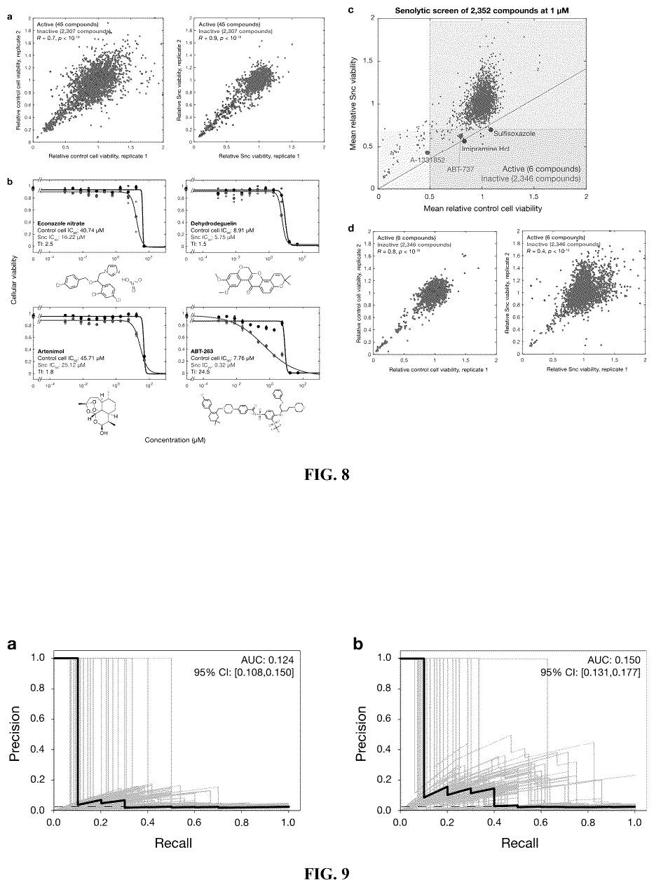

Validation dose-response measurements are shown in FIG. 8. Three-fold serial dilutions of compound were prepared in DMSO, then added to cell plates (final concentration of DMSO. 1%). Cell viability was then assayed as detailed above, and resazurin fluorescence values were linearly interpolated with respect to values from empty and positive control (non-treatment) wells. Experiments were performed in biological duplicate each on two independent occasions. When possible, chemical stocks from commercial suppliers were used: econazole nitrate (Cayman Chemical 20223), artenimol (Cayman Chemical 19846), and ABT-263 (Cayman Chemical 11500) were from Cayman Chemical, and dehydrodeguelin was aliquoted from the screening library.

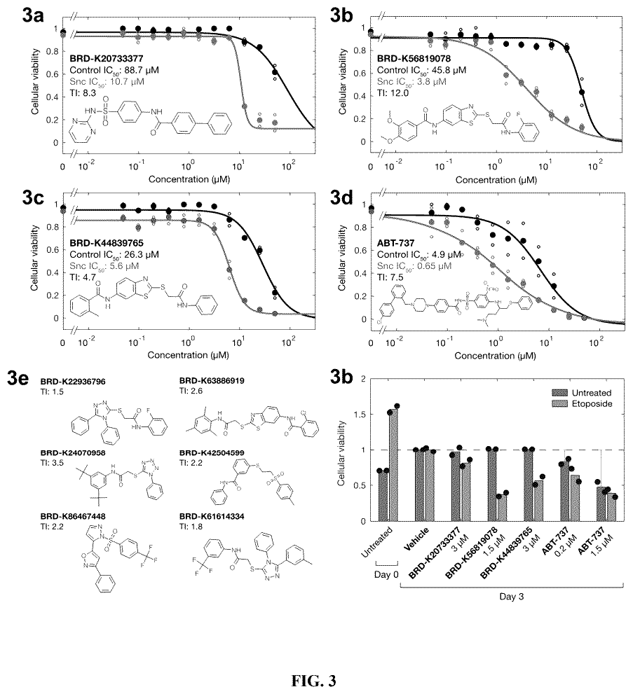

Calculation of cellular viability values in dose-response curves. To determine relative cell viability values for the dose-response curves shown in FIGS. 3, 4, 8, 11, and 13, resazurin fluorescence intensity measurements were measured after 3 days of compound treatment and 1 day of resazurin incubation. For each sample, the fluorescence intensity value was normalized by the mean of two untreated control values. Cellular viability values are therefore indicated as fractions of the cellular viability of untreated controls. Vehicle (1% DMSO) control values are included in each dose-response curve. Experiments were performed in biological duplicate each on two independent occasions.

Calculation of baseline cellular viability values. As shown in Panel 3f of FIG. 3, cellular viability was calculated as above for untreated IMR-90 cells and etoposide-treated IMR-90 cells, which were treated with DMSO or compounds for 3 days and then incubated in the presence of resazurin for 1 day. Additionally, baseline cellular viability values were calculated for untreated cells at the start of the experiment. Resazurin was added to a final concentration of 0.15 mM on the day of seeding (corresponding to day 0 in FIG. 7). Cells were incubated for 1 day, then fluorescence intensity values were read. These fluorescence intensity values were normalized by the mean of two vehicle (1% DMSO)-treated control values at day 3, such that cell proliferation between days 0 and 3 in the presence of DMSO vehicle is indicated by an increase in cellular viability values. Experiments were performed in biological duplicate on one independent occasion.

Curve-fitting and estimation of IC₅₀ values. To estimate IC₅₀ values in dose-response curves, nonlinear least-squares fitting (the Isqcurvefit function in MATLAB, ver. R2019b) was used to fit relative growth values to Hill functions of the form

$$H ( x ) = {b}_{0} + \frac{m {x}^{β}}{{x}_{0.5}^{β} + {x}^{β}} ,$$

while enforcing H≥0) for all x. IC₅₀ values were determined by numerically solving the best-fit Hill function for x given H(x)=0.5.

Deep learning model. The deep learning model used builds on that applied in Stokes. J. M, et al. Cell 2020, 180 (4). 688-702, which is incorporated herein by reference in its entirety, and uses Chemprop (github.com/chemprop/chemprop), a software package for molecular property prediction that implements the graph neural networks described below and in the main text. For each compound, a graph-based molecular representation was generated from the compound's simplified molecular-input line-entry system (SMILES) string using RDKit (ver. 2021.09.01). A feature vector for each atom and bond in the compound was generated based on the following computable features:
1. atom features including the atomic number, number of bonds for each atom, formal charge, chirality, number of bonded hydrogen atoms, hybridization, aromaticity, and atomic mass;
2. bond features including the bond type (single, double, tripe, or aromatic), conjugation, ring membership, and stereochemistry.

The model implements the bond-based message-passing convolutional neural network. Here, each message (a real number) associated with a bond was updated by summing the messages from neighboring bonds, concatenating the current bond's message with the sum, and applying a single neural network layer with a non-linear activation function. After a fixed number of message-passing steps, the messages across the molecule were summed to produce a final message representing the molecule. This message was inputted into a feed-forward neural network, which output a final prediction of the compound's senolytic activity. The final output was a real number between 0 (is not senolytic) and 1 (is senolytic), describing the probability that the compound was predicted to be senolytic.

Model optimization. Three model optimizations were used to improve model performance. First. 200 molecule-level features computed with RDKit were added to the graph-based representation of each compound. This step was performed in order to provide additional information about predicted global properties of each compound, augmenting the local message-passing approach. Second. Bayesian hyperparameter optimization was used in order to select hyperparameters for the model. Doing so using Chemprop's hyperopt function, the following hyperparameters were found and used for all Chemprop models: depth. 2: dropout. 0 number of feedforward layers. 3: hidden size. 600. Finally, ensembling was used to increase the robustness of Chemprop model predictions, as detailed separately for Model evaluation and Model predictions and filtering below.

Model evaluation. Each compound in the initial training dataset of 2.352 compounds was assigned a binary activity value of 0 (no senolytic activity) or 1 (possesses senolytic activity), as shown in Panel 1d of FIG. 1. To evaluate model performance using the auPRC, the initial training dataset was partitioned such that 80% of the compounds (1.882 compounds) were reserved for training and validation and 20% of compounds (470 compounds) were withheld for testing and calculation of PRCs. Active compounds in each group were distributed similarly as in the overall dataset (10 of 470 compounds, or 2.1%). For each Chemprop model, training was performed for 30 epochs using random 80-10⁻¹⁰ training-validation-testing splits of the training subset, with each model being assigned a different random seed. By default, the binary cross entropy was used as the loss function. Ten models were then pooled together to form an ensemble. This ensemble of models was applied to the withheld testing subset, and prediction scores of the ensemble were taken as the average of the prediction scores of all 10 models in the ensemble. Precision-recall curves were generated by comparing the prediction score to the withheld activity value for each compound in the testing subset using scikit-learn.

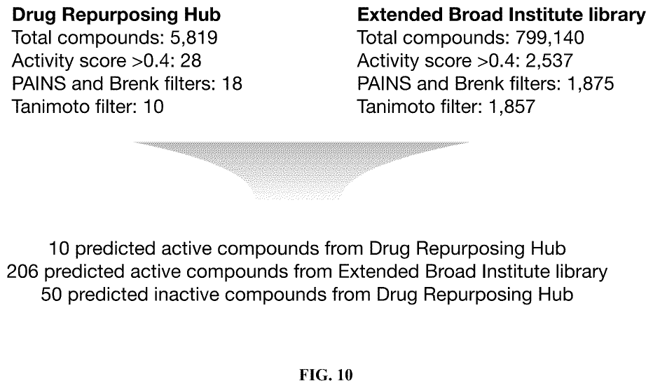

Random forest classifiers were independently trained using scikit-learn. The same training and withheld test sets as above were used, and an exhaustive hyperparameter grid search was performed. 360 random forest models were trained for hyperparameters in the following combinatorial space: max depth between 5 to 40, in intervals of 5: number of estimators between 20) and 100, in intervals of 20; max features between 20 and 180, in intervals of 20. Precision-recall curves were generated using scikit-learn as above. For both Chemprop and random forest models, bootstrapping with 100 subsamples, each subsample with size equal to the test set, was used to calculate 95% confidence intervals for the auPRC and bootstrapped variations of precision-recall curves (Panel 1e of FIG. 1 and FIG. 9).

t-SNE and visualization, sklearn, manifold's TSNE function was used in conjunction with Morgan fingerprint representations of all compounds (radius=2 and number of bits=2048) to visualize compounds (Panel 1g of FIG. 1). The Jaccard distance, also known as the Tanimoto distance, was used as the distance metric: the Tanimoto distance is defined as Tanimoto distance =1-Tanimoto similarity, and the Tanimoto similarity between two fingerprints is given by the quotient of the number of 1-bits in the intersection of both fingerprints divided by the number of 1-bits found in their union. Calculations of Tanimoto similarity as described herein were generally based on Morgan fingerprint representations of all compounds (radius=2 and number of bits=2.048). The choice of the Jaccard metric implies that the distance between points reflects the Tanimoto similarity of the corresponding compounds, with greater t-SNE distance corresponding to lower Tanimoto similarity. A perplexity parameter of 10 was used to generate plots with well-spaced data points.

Model predictions and filtering. For the final model. 20 Chemprop models were each trained for 30 epochs using random 80-10⁻¹⁰ training-validation-testing splits. The models were then deployed to predict the senolytic activities of 804.959 compounds comprising the Broad Institute's Drug Repurposing Hub (with 5819 unique compounds scored) and an extended Broad Institute library of 799.140 compounds. For each compound, the prediction scores of all models were averaged to determine the final prediction score for the compound. Following prediction of senolytic activity, compounds with high prediction scores (>0.4) possessing PAINS and Brenk substructures were filtered out using rdkit. Chem's FilterCatalog package. The remaining compounds with high prediction scores were filtered based on the Tanimoto similarity, and all calculations of Tanimoto similarity were performed as described above.

Chemical curation. ABT-737 was from Cayman Chemical (Cayman Chemical 11501). Other compounds were procured from the Broad Institute Center for the Development of Therapeutics. ChemBridge, and May bridge.

Calculation of physicochemical properties. For each compound, the physicochemical properties shown in Table 1 herein below were calculated from the corresponding SMILES string using SwissADME.

Measuring cytotoxicity against HepG2 and HEK293 cells. Cytotoxicity in human embryonic kidney (HEK293) and liver carcinoma (HepG2) cells was assayed as above using a resazurin assay. Cells were obtained from ATCC (ATCC CRL-1573 and HB-8065), passaged <10 times, and grown in Dulbecco's modification of Eagle's Medium (DMEM: Corning 10⁻⁰¹³-CV) supplemented with 10% FBS and 1% penicillin-streptomycin. 99 μL of cells were plated into each well of a 96-well clear flat-bottom, black polystyrene tissue-culture-treated plate at a density of ˜10+ cells/well, and plates were incubated overnight for adhesion. The day after plating. 1 μL of each compound, prepared as two-fold serial dilutions of 5 mM stock solutions in DMSO, was added to each well. Cells were incubated for 3 days, after which resazurin was added to each well to a final concentration of 0.15 mM. After an additional 1 day of incubation, the fluorescence excitation/emission at 550/590 nm was read using a SpectraMax M3 plate reader. Experiments were performed in biological duplicate. IC₅₀ values were determined by normalizing with respect to the fluorescence intensity values of untreated control cells on day 3, as described in Calculation of cellular viability values in dose-response curves above.

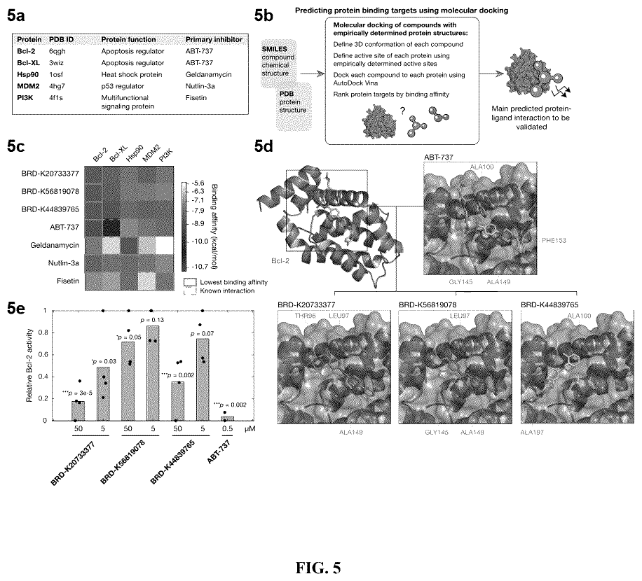

Molecular docking simulations. Molecular docking simulations were performed using AutoDock Vina 1.2.0. Compounds were provided as SMILES strings and represented in three dimensions using OpenBabel. Protein structures were curated from the PDB using the accession codes tabulated in Panel 5a of FIG. 5, and the coordinates of the bound inhibitors were retrieved using PyMOL to define the active site of each protein (as detailed further below). Next. AutoDockTools (ver. 1.5.7) was used to prepare each protein and compound for docking by converting each file into AutoDock Vina's PDBQT format. For compound preparation, hydrogen atoms were added at pH 7.4, and hydrated docking was used whenever possible. For protein preparation, the default prepare_receptor command was used. For each protein, the active site was based on the bounding box of the corresponding bound inhibitor from the PDB: the center of the active site was taken to coincide with the center of the bounding box, and the length of each edge of the bounding box was multiplied by a factor of 1.5× to specify the corresponding edge of the active site, in order to allow for broader conformational sampling. Docking was performed with a default exhaustiveness of 32, which specifies the number of runs that start with a random ligand conformation, and a default n poses of 20, which specifies the final number of ligand poses to report. All binding affinities predicted by docking simulations are reported in Panel 5c of FIG. 5. The predicted bound conformations shown in Panel 5d of FIG. 5 were visualized using PyMOL (ver. 2.5.2).

BCL-2 TR-FRET. Inhibition of Bcl-2 binding to a peptide ligand was measured using the BCL-2 TR-FRET Assay Kit from BPS Bioscience (BPS Bioscience 50222). Briefly, the provided BCL TR-FRET assay buffer was diluted 1:3 with ultrapure Milli-Q water. The anti-His terbium-labeled donor and dye-labeled acceptor were each diluted 1:100 with diluted assay buffer. The BCL-2 peptide ligand was thawed on ice and diluted 1:40 with diluted assay buffer, and BCL-2 protein was diluted with diluted assay buffer to a working concentration of 1 ng/μL. Test compounds at the indicated final concentrations were prepared as stock 10% DMSO solutions in ultrapure Milli-Q water. The reaction was performed by combining 5 μL diluted donor. 5 μL diluted acceptor. 2 μL test compound. 5 μL diluted ligand, and 3 μL diluted protein in each well of the provided white, flat-bottom 384-well plate. Positive control reactions had 2 μL 10% DMSO in water in lieu of test compound. Negative control reactions had 2 μL 10% DMSO in water and 5 μL diluted assay buffer in lieu of test compound and diluted ligand. Reactions were incubated at room temperature for 3 hours, and fluorescent intensities were measured using a SpectraMax® M5 plate reader and manufacturer software (SoftMax Pro 6) with the following TR-FRET settings; for terbium-labeled donor emission, ex/em. 340/620 nm: lag time. 100 μs: integration time. 500 μs: for dye-acceptor emission, ex/em. 340/665 nm: lag time. 100 μs; integration time. 500 μs. The TR-FRET ratio. 665 nm emission/620 nm emission, was calculated for all reactions, and percentage activity was calculated by linearly interpolating the positive and negative control TR-FRET ratio values between relative activity values of 0) and 1. Experiments were performed in biological duplicate and repeated on independent occasions.

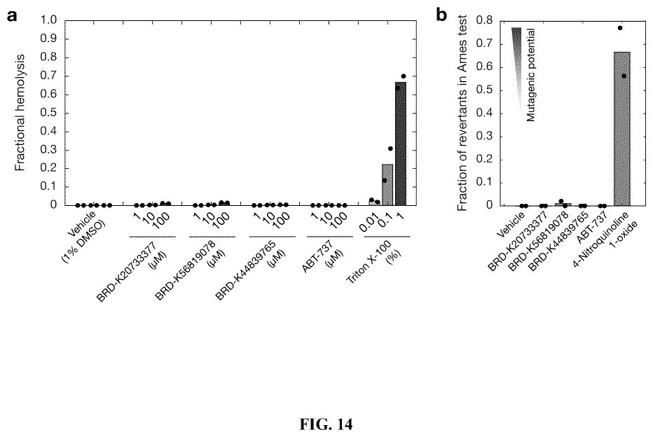

Hemolysis assay. Whole human blood containing EDTA (Innovative Resarch IWB1K2E) was centrifuged at 120×g at 4° C., for 5 min and resuspended in Dulbecco's PBS (DPBS: VWR 02-0119-0500). These washing steps were repeated until the supernatant was clear. Red blood cells were then resuspended in DPBS to 5×10⁸ cells/mL, and 100 μL of cells was plated into each well of a 96-well clear round-bottom plate. Compounds were added to the indicated concentrations, and DMSO was used as a vehicle. Samples were incubated for 1 h at 37° C., without shaking, after which plates were centrifuged at 1500×g at room temperature for 5 min to pellet cells. 60 μL of the supernatant from each sample was then transferred to a 96-well clear flat-bottom plate, and the absorbance was read at 405 nm using a SpectraMax M3 plate reader to quantify the amount of soluble hemoglobin. Fractional hemolysis was determined by linearly interpolating absorbance values with respect to a positive control (saturation with 10% Triton X-100) and a negative control (1% DMSO vehicle). Results are depicted in FIG. 14.

Genotoxicity assay. An Ames 384-ISO test (6041-1S) from Environmental Bio-Detection Products, Inc, was used following the manufacturer's instructions. Briefly. Salmonella typhimurium TA100 was grown overnight (16-18 h) at 37° C., with shaking at 300 rpm and treated with the provided exposure media and compound samples at the final concentrations indicated. Treatment with the provided 4-nitroquinoline 1-oxide (4NQO), a mutagen, was used as a positive control. Cells were then added to the provided reversion solution, and each sample was split into 48 wells of 384-well plates. Plates were incubated at 37° C., for 2 days, after which the number of revertant (yellow-colored) wells corresponding to each sample was counted. Additionally, we verified that each test compound did not inhibit the growth of S, typhimurium TA100. An overnight bacterial culture was diluted 1:10.000 in LB medium (Becton Dickinson 244620) and plated using 99 μL working volumes into the wells of a clear flat-bottom 96-well plate. One μL of two-fold dilutions of each test compound in DMSO, starting from a final concentration of 500 μM, was added across wells, and plates were sealed and incubated overnight at 37° C., to determine bacterial growth. Results are shown in FIG. 14.

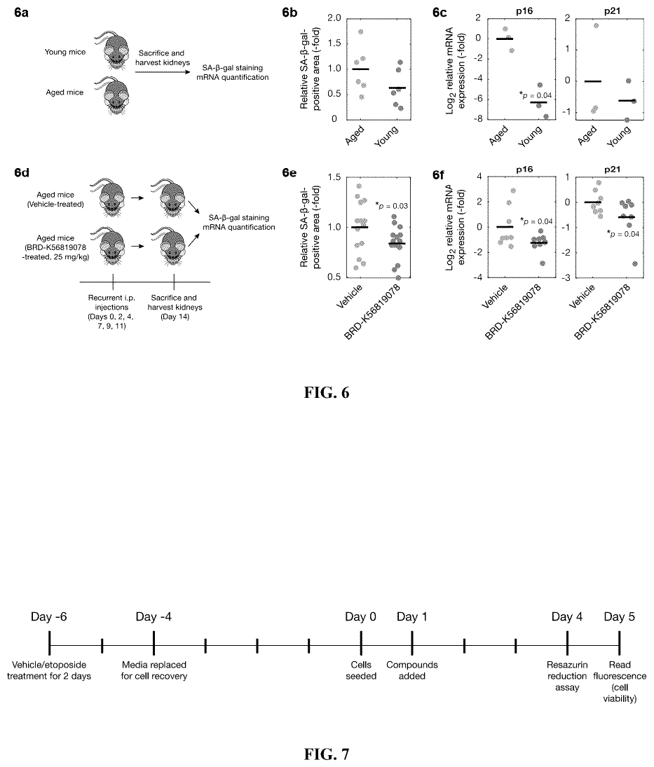

Aged mouse model experiments. For the baseline experiment, the results of which are shown in Panels 6a-6e of FIG. 6, young (6- to 8-week-old) and aged (90-week-old) female C₅₇BL/6J mice were procured from The Jackson Laboratory and quarantined at least 2 days prior to use. For the experiment the results of which are shown in Panels 6d-6f of FIG. 6, aged (80-week-old) female C57BL/6J mice were procured from The Jackson Laboratory and quarantined at least 2 days prior to use. Animals were housed in a facility maintained at 20-26° C., ambient temperature. 40-65% relative humidity, and a 12:12 light-dark cycle. Enrichment devices were included in the animal environments as required and changed bi-weekly. All mice were treated in accordance with protocol IS00000852-6, approved by Harvard Medical School Institutional Animal Care and Use Committee and the Committee on Microbiological Safety.

For compound administration. BRD-K56819078 (ChemBridge 7507010) was prepared fresh in 10% DMSO: 45% PEG300:45% water for injection w/w, and for each mouse a total of six intraperitoneal injections over a 14-day period were performed at 25 mg/kg per injection, as determined by weighing each mouse immediately prior to injection. All mice were euthanized by CO₂ asphyxiation and dissected, and one kidney was harvested per mouse. Each kidney was divided for SA-β-gal staining and mRNA measurements. Samples that were designated for SA-β-gal staining were embedded in OCT medium and flash-frozen in liquid nitrogen. Samples that were designated for mRNA measurements were placed in RNAlater™ Stabilization Solution (Thermo Fisher AM7021) and flash-frozen with dry ice.

For SA-β-gal staining, kidney samples were oriented and cut into 10 μm-thin sections using a Leica CM1950 cryostat. SA-β-gal staining was then performed similarly to the above, but with modifications. Frozen sections were fixed for 15 minutes using 2% formaldehyde and 0.2% glutaraldehyde in PBS (pH 7.4). Sections were then washed in PBS and incubated overnight at 37° C., in a dry incubator with β-gal staining solution, an aqueous solution containing 40 mM citric acid/sodium phosphate (MilliporeSigma C0759 and S9763). 5 mM potassium ferrocyanide (MilliporeSigma P9387). 5 mM potassium ferricyanide (MilliporeSigma P8131). 2 mM magnesium chloride (MilliporeSigma M8266). 150 mM sodium chloride (Fisher Scientific S271), and 1 mg/mL X-gal (MilliporeSigma 9660), titrated to pH 6.0. Sections were washed in PBS, counterstained with Nuclear Fast Red (VWR AAJ61010-AP) for 5 min at room temperature, then washed again in PBS before imaging. Imaging was performed on an EVOS XL Core or a Leica DMil equipped with a Flexacam CI camera. Two fields of view were captured for each kidney section, the images were thresholded by color using ImageJ ver. 2.0.0-rc-69/1.52p (National Institutes of Health), and the ratios of blue area (SA-β-gal-positive area) to total red and blue area (all cells) were calculated for each field of view. One kidney sample from each of the vehicle- and BRD-K56819078-treated aged mice groups failed to stain for SA-β-gal, which may arise if the sections did not contain any kidney cortical region: data from these samples were discarded.

For mRNA measurements, kidney samples were homogenized using an SP Bel-Art ProCulture® cordless homogenizer, and mRNA was extracted and quantified as described above in mRNA quantification using quantitative PCR, using a PureLink™ RNA Mini Kit (Thermo Fisher 12183020) for extraction. The following primers were used, and the relative expression of each gene was determined by normalization to β-actin expression for each sample:

p16 Forward primer:
AGGGCCGTGTGCATGACGTG
p16 Reverse primer:
GCACCGGGCGGGAGAAGGTA
p21 Forward primer:
AACATCTCAGGGCCGAAA
p21 Reverse primer:
TGCGCTTGGAGTGATAGAAA
β-actin Forward primer:
GGCTGTATTCCCCTCCATCG
β-actin Reverse primer:
CCAGTTGGTAACAATGCCATGT

Two-sided, two-sample unpaired 1-tests or one-way ANOVA tests were performed using MATLAB, as shown in Panel 1c of FIG. 1, Panel 4b of FIG. 4, and FIG. 13 to test the hypothesis that mRNA expression values for p16, p21, and KI67 were different from control (vehicle-treated or early passage) cell values in the therapy-induced and replicative senescence models. Two-sided, two-sample unpaired t-tests were performed using MATLAB as shown in Panel 5e of FIG. 5 to test the hypothesis that the Bcl-2 activity values in each treatment condition had mean values different from that of corresponding positive control measurements (relative Bcl-2 activity values of 1). One-sided, two-sample permutation tests for differences in mean value were performed using MATLAB as shown in Panels 6c, 6e, and 6f of FIG. 6 to test the hypothesis that SA-β-gal-positive areas or mRNA expression values for p16 and p21 were different from control (naïve aged mice or vehicle-treated aged mice) values for mouse model experiments. Exact permutation tests, in which all possible combinations were considered, were used for all comparisons with the exception of that depicted in Panel 6e of FIG. 6, for which 100,000 random combinations were used due to the larger sample sizes. Where relevant, data distribution was assumed to be normal.

TABLE 1
Physicochemical properties and cytotoxicity of identified compounds.

| Compound | BRD-K20733377 | | BRD-K56819078 | | BRD-K44839765 | | ABT-737 | |
|---|---|---|---|---|---|---|---|---|
| Canonical | C1═CC═C(C═C1) | | COC1═C(C═C(C═C1) | | CC1═CC═CC═C1C | | CN(C)CCC(CSC1═CC═CC═C1) | |
| simplified | C2═CC═C(C═C2) | | C(═O)NC2═CC3═C(C═C2) | | (═O)NC2═CC3═C | | NC2═C(C═C(C═C2)S(═O) | |
| molecular-input | C(═O)NC3═CC═C | | N═C(S3)SCC(═O) | | (C═C2)N═C(S3)SCC | | (═O)NC(═O)C3═CC═C | |
| line-entry system | (C═C3)S(═O)(═O) | | NC4═CC═CC═C4F)OC | | (═O)NC4═CC═CC═C4 | | (C═C3)N4CCN(CC4) | |
| (SMILES) string | NC4═NC═CC═N4 | | | | | | CC5═CC═CC═C5C6═CC═C | |
| | | | | | | | (C═C6)Cl)[N+](═O)[O—] | |
| Molecular weight | 430.48 | Da | 497.56 | Da | 433.55 | Da | 813.43 | Da |
| Number of heavy | 31 | | 34 | | 30 | | 56 | |
| atoms | | | | | | | | |
| Number of | 7 | | 10 | | 8 | | 17 | |
| rotatable bonds | | | | | | | | |
| Topological polar | 109.43 | Å² | 143.09 | Å² | 124.63 | Å² | 164.49 | Å² |
| surface area | | | | | | | | |
| (TPSA) | | | | | | | | |
| Lipinski- | Yes | | Yes | | Yes | | No; | |
| conforming | | | | | | | MW > 500 g/mol, | |
| | | | | | | | TPSA > 140 Å² | |
| Veber-conforming | Yes | | No; | | Yes | | No; number of | |
| | | | TPSA > 140 Å² | | | | rotatable | |
| | | | | | | | bonds > 10, | |
| | | | | | | | TPSA > 140 Å² | |
| Pan-assay | None | | None | | None | | None | |
| interference | | | | | | | | |
| compounds | | | | | | | | |
| (PAINS) | | | | | | | | |
| Brenk | None | | None | | None | | Yes; nitro group | |
| substructures | | | | | | | and oxygen- | |
| | | | | | | | nitrogen single | |
| | | | | | | | bond | |
| HEK293 IC₅₀ | 51.1 | μM | 21.4 | μM | 159.5 | μM | 28.8 | μM |
| HepG2 IC₅₀ | 70.3 | μM | 420.7 | μM | 382.6 | μM | 131.9 | μM |

For comparison, values for ABT-737 are shown. Lipinski-conforming indicates that a compound violates no more than one of Lipinski's rule of five: (a)≤5 hydrogen bond donors, (b) ≤10 hydrogen-bond acceptors, (c) molecular weight <500 Da, and (d) log P partition coefficient <5. Veber-conforming indicates that a compound violates none of Veber's rules for oral bioavailability: (a)≤10 rotatable bonds and (b) TPSA≤140 Å2. IC₅₀ values for human embryonic kidney (HEK293) and human liver carcinoma (HepG2) cells represent values inferred from curve-fitting with data from two biological replicates, as detailed elsewhere herein.

### Chemical Screens of 2,352 Compounds Identify Senolytics.

To identify compounds with senolytic activity used to train the model. 2.352 compounds, largely from a library of FDA-approved drugs and drugs undergoing clinical trials, were screened for senolytic activity in a model of therapy-induced senescence as described above. Human lung (IMR-90) fibroblasts were treated with etoposide to induce senescence via the formation of double-stranded DNA breaks, and cells were allowed to recover in etoposide-free medium. Senescence was confirmed by staining for senescence-associated β-galactosidase (SA-β-gal) at timepoints corresponding to one day before compound treatment and the day of compound treatment, which indicated substantively increased straining in etoposide-treated Sncs relative to vehicle (0.5% dimethyl sulfoxide)-treated controls (Panel 1b of FIG. 1). Complementing these observations of SA-β-gal staining, p16, p21, and KI67 mRNA levels were quantified using quantitative PCR (Panel 1c of FIG. 1). Increased p16 and p21 mRNA levels are associated with senescence, and decreased mRNA levels of KI67, a proliferation marker, are associated with growth arrest. The measurements indicated at least one-fold increases (p16 and p21) and decreases (KI67) in mRNA levels in Sncs relative to vehicle-treated controls (Panel 1c of FIG. 1). Sncs were then treated with 10 μM of each compound, and relative viability was measured by reduction of resazurin, a metabolic indicator, after a three-day course of treatment (FIG. 7). As controls, vehicle-treated cells were counter-screened in the same way.

As a starting point for senolytic activity, active compounds were defined as those for which relative Snc viability was <0.5, relative viability of control cells was >0.5, and the ratio of relative Snc/control viability was <0.7 (Panel 1d of FIG. 1). Requiring the relative viability of control cells to be >0.5 ensured that active compounds were not strongly cytotoxic, while requiring the ratio of relative Snc/control viability to be <0.7 ensured that active compounds have at least moderate selectivity against Sncs. With these criteria. 45 compounds emerged as active from the initial screens (Panel 1d of FIG. 1). These compounds included documented senolytics such as ABT-737. ABT-263. A-1331852. A-1155463, and ouabain. A subset of the active compounds was further validated by performing detailed dose-response measurements, which demonstrated that these compounds exhibit therapeutic indices against Sncs—that is, the ratio of Snc to control cell IC₅₀ values-ranging from 1.5 (weakly selective) to 24.5 (selective) and IC₅₀ values against Sncs ranging from 0.32 to 25.1 μM (FIG. 8). To explore the effects of different screening concentrations on the identification of active compounds, the screen was repeated with treatment at 1 μM and significantly fewer-only six-active compounds were found, which did not include documented senolytics (FIG. 8).

Based on the foregoing criteria for active compounds, a screen at 10 μM revealed more active compounds, including documented senolytics, than the screen at 1 μM. The screening results at 10 μM were thus used to train subsequent models, including a binary classifier.

### Design and Validation of Graph Neural Network Models

The screening data was then used to train deep learning models that predict senolytic activity based on chemical structure. Message-passing graph neural network (MPNN) models were trained and deployed to predict senolytic activity based on chemical structure as described herein above. MPNNs are a type of supervised model that takes as input a chemical structure of a molecule, integrates local information contained at each atom and bond, and produces as output a prediction score representing the probability that the molecule possesses a property of interest. The ability of these models to predict senolytic activity was assessed by training and testing on 80-20 splits of the screening data. Precision-recall curves, which plot the true positive rate against the positive predictive value (Panel 1e of FIG. 1), were generated. The area under the precision-recall curve (auPRC), which measures the ability of the model to correctly identify a senolytic compound, was favorable, with a value of 0.24 (95% confidence internal: [0.14, 0.34]). This indicates that the model could more accurately identify senolytic compounds in the training set as compared to random (auPRC of 0.019). In contrast, alternative models based on random forests resulted in reduced performance, with at most an auPRC of 0.15 (FIG. 9). Additional benchmarks of model performance using different metrics, including the positive predictive value at different prediction score thresholds, similarly indicated better performance in the graph neural network model.

The model was then retrained using the entire screening dataset, and it was applied to predict the senolytic activities of 804.959 compounds comprising the Broad Institute's Drug Repurposing Hub (with 5819 unique compounds scored) and an extended Broad Institute library of 799.140 compounds. The compounds exhibited a range of prediction scores, from 2.1×10⁻⁶ to 0.70 (Panel 1f of FIG. 1), suggesting that the model was able to discriminate between predicted active and inactive compounds. Model predictions were also structurally diverse, as indicated by t-distributed stochastic neighborhood embedding (t-SNE) visualization. Compounds corresponding to closer points are more structurally similar, and the plot revealed that the chemical spaces covered by high-ranking compounds from the Drug Repurposing Hub and the extended Broad Institute library are similar, but extend beyond, that of active compounds from the screen (Panel 1g of FIG. 1). Furthermore, high-ranking compounds were largely separated from low-ranking compounds, indicating that the model demonstrates discriminatory ability.

The search space was narrowed by applying filters selective for favorable medicinal chemistry properties and structural novelty (FIG. 10). First, compounds with promiscuously reactive substructures (PAINS) and pharmacokinetically unfavorable substructures (Brenk substructures) were filtered out. Second, the Tanimoto similarity was used to curate a structurally diverse set of compounds. The Tanimoto similarity a set-based measure of similarity that has a value of 1 when two compounds are identical and a value of 0 if two compounds have no substructure in common. Of the remaining compounds, only those with low Tanimoto similarity (≤0.5) to any compound in the training dataset were retained. From the filtered compounds, we all 10 compounds with prediction scores >0.4 from the Drug Repurposing Hub, and 206 in-stock compounds with prediction scores >0.4 from the extended Broad Institute library were curated. As a negative control, the bottom-ranking 50 filtered compounds, with prediction scores <7× 10⁻⁵, from the Drug Repurposing Hub were also curated.

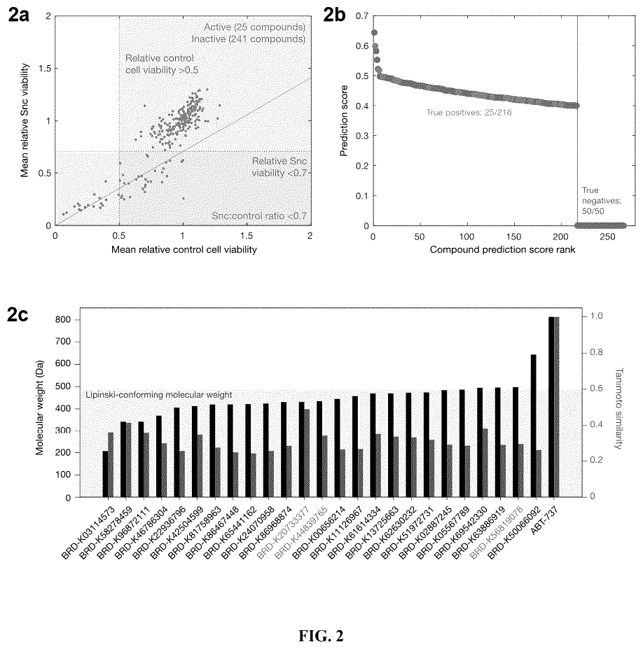

Measuring senolytic activity as before, the preliminary screens revealed that 25 of the 216 curated high-ranking compounds were active, in contrast to none of the 50 curated low-ranking compounds (Panels 2a and 2b of FIG. 2). The working hit rate (positive predictive value) of the approach was 11.6%, suggesting that the platform enriched for active compounds relative to the initial screen (1.9% baseline enrichment), and that improvements to the model may further increase prediction accuracy. The low false negative rate of the approach also suggests that the model can contribute to filtering out molecules that likely do not possess senolytic activity in high-throughput screens. Notably, nearly all validated hits possessed Lipinski-conforming molecular weights of <500 Da and were structurally dissimilar to all molecules in the training set, with Tanimoto similarity scores between 0.24 and 0.49 (Panel 2c of FIG. 2). This indicated that the hit compounds were drug-like and structurally novel.

### Validation of Compounds in Models of Therapy-Induced Senescence

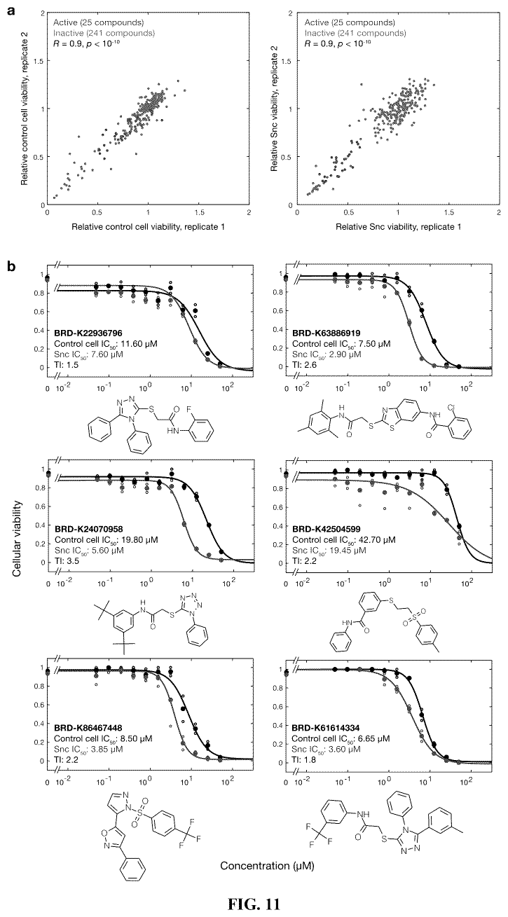

The dose-response of several compounds that were particularly selective at 10 μM was examined in detail: of these, three compounds. BRD-K20733377. BRD-K56819078, and BRD-K44839765, exhibited encouraging therapeutic indices of 8.3, 12.0, and 4.7, respectively, which were at least comparable to the therapeutic index of ABT-737 (7.5: Panels 3a-3e of FIG. 3 and FIG. 11). Measurements of cellular viability across time indicate that all three compounds could selectively kill Sncs without inhibiting the growth of control cells at selective concentrations, in contrast to ABT-737 (Panel 3f of FIG. 3). Cellular viabilities of control, non-senescent cells were increased after three days of incubation with 1% dimethyl sulfoxide (DMSO) as a vehicle, demonstrating proliferation of control cells: in contrast, viabilities of etoposide-treated Sncs were decreased, an effect which could arise from post-treatment etoposide lethality on the timescale of the experiments. Nevertheless, as compared to DMSO-treated Sncs and control cells, treatment with BRD-K20733377. BRD-K56819078, and BRD-K44839765 at concentrations between 1.5 to 3 μM resulted in the selective elimination of Sncs and no difference in the viabilities of control cells. In contrast, treatment with ABT-737 resulted in decreases in the viabilities of control cells, by ˜20 to 50%, at concentrations including 0.2 and 1.5 μM (Panel 3f of FIG. 3).

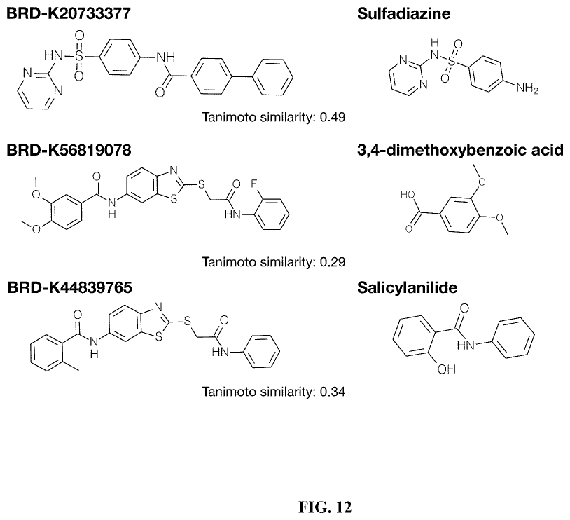

Given that the identified compounds may exhibit more promising selectivity than ABT-737, their structural and physicochemical properties were investigated in greater detail. All three compounds are drug-like compounds from the extended Broad Institute library with no current clinical use. BRD-K56819078 and BRD-K44839765 share a benzothiazole-containing substructure, and all three compounds occupy a chemical space distinct from that of the training dataset, with the closest compounds being sulfadiazine. 3.4-dimethyoxybenzoic acid, and salicylanilide, as measured by Tanimoto similarity (FIG. 12). Importantly, all three compounds are Lipinski-conforming and possess 7 to 10 rotatable bonds and topological polar surface areas between 109 and 143 A2 (Table 1). These chemical properties suggest that BRD-K20733377 and BRD-K44839765 can be orally bioavailable, as they satisfy the Veber criteria of ≤10 rotatable bonds and topological polar surface area of ≤140 A2. Additionally, the 143 A2 topological polar surface area of BRD-K56819078 is only slightly larger than the 140 A2 threshold. In contrast. ABT-737 possesses a molecular weight of 879.5 Da. 17 rotatable bonds, and a topological polar surface area of 164 A2.

To further investigate the senolytic activities of BRD-K20733377. BRD-K56819078, and BRD-K44839765, the selectivity of these compounds against Sncs was verified in in an IMR-90 model of therapy-induced senescence using doxorubicin (FIG. 13). SA-β-gal staining and quantification of p16, p21, and KI67 mRNA levels revealed that doxorubicin-treated wells exhibited a senescence phenotype similar to that of etoposide-treated cells, and all three compounds were indeed selective against doxorubicin-treated cells, with therapeutic indices between 4.3 and 7.3 and IC₅₀ values≤12.1 μM (FIG. 13). Furthermore. IC₅₀ values for all three compounds were ≥20 μM for human embryonic kidney (HEK293) and liver carcinoma (HepG2) cells (Table 1), suggesting that all three compounds may not be strongly nephrotoxic or hepatotoxic at concentrations that are selective against Sncs, and that the compounds might target pathways specifically involved in cellular senescence.

### Validation of Compounds in a Model of Replicative Senescence

Orthogonal to models of therapy-induced senescence, the efficacy of BRD-K20733377. BRD-K56819078, and BRD-K44839765 in a model of replicative senescence was measured. Early- and late-passage IMR-90 cells were cultured, and late-passage cells were passaged until they became non-dividing. To confirm senescence of late-passage cells. SA-β-gal staining (Panel 4a of FIG. 4) and quantitation of p16, p21, and KI67 mRNA levels (Panel 4b of FIG. 4) were performed as described herein above: this revealed substantive SA-β-gal staining in late-passage cells and similarly increased or decreased p16, p21, and KI67 mRNA levels relative to those in the etoposide model. Treating early- and late-passage cells with each compound, it was found that all three compounds were selective against late-passage Sncs (Panels 4c-4f of FIG. 4). BRD-K20733377 and BRD-K56819078 were similarly selective against late-passage Sncs than ABT-737, while BRD-K44839765 was less-largely consistent with the findings for etoposide-treated Sncs (Panels 3a-3d of FIG. 3). The IC₅₀ values of all compounds were generally increased in early-passage cells as compared to vehicle-treated controls in the etoposide model, and in late-passage cells as compared to etoposide-treated Sncs. Despite differences in IC₅₀ values, these findings consistently support the selectivity of the identified compounds in a model of replicative senescence.

### Molecular Docking and TR-FRET Study of Identified Compounds

Given that BRD-K20733377. BRD-K56819078, and BRD-K44839765 are selective against Sncs in different models of senescence, it was hypothesized that they may act on targets conserved in senescence pathways. As a starting point for determining their potential mechanisms of action, attention was to focused on documented senolytic protein targets, including Bcl-2 and Bcl-2 family proteins, heat shock proteins such as Hsp90, and proteins involved in cell cycle regulation such as MDM2 and PI3K (Panel 5a of FIG. 5). Bcl-2 and Bcl-2 family proteins, including Bcl-XL, regulate cell death by apoptosis and are selectively inhibited by several senolytics, including ABT-737. ABT-263, and A-1331852. Hsp90 is a ubiquitously expressed chaperone and stress response protein that stabilizes various client proteins including those involved in oncogenesis and apoptosis, of which Akt aids in preventing apoptosis in Sncs. Hsp90 inhibitors, including geldanamycin and 17-DMAG, have been identified as effective senolytics. MDM2 directly binds to the transactivation domain of p53 and inhibits its transcriptional activity, an interaction that also results in the ubiquitination and proteomic degradation of p53, and senolytics including nutlin-3a and UBX0101 bind MDM2, inhibit the MDM2-p53 interaction, and increase the availability of pro-apoptotic p53. PI3K activation phosphorylates and activates Akt, which, as mentioned above, prevents apoptosis in Sncs, and senolytics including fisetin and quercetin bind PI3K.

Molecular docking simulations were performed to predict likely protein targets of BRD-K20733377. BRD-K56819078, and BRD-K44839765 (Panel 5b of FIG. 5). As controls, documented ligands of Bcl-2. Bcl-XL. Hsp90. MDM2, and PI3K (Panel 5a of FIG. 5) were included, and the binding of each molecule to each of these proteins was simulated with AutoDock Vina. The protein structure of each target was obtained from a corresponding complex with a bound inhibitor from the Protein Data Bank (PDB), and the active site of each protein structure was defined based on the conformation of the corresponding bound inhibitor. Representing each chemical compound in three dimensions, docking each compound into the active site of each structure resulted in a range of predicted binding affinity values (Panel 5c of FIG. 5). Notably, taking the protein-ligand interaction with the lowest binding affinity (highest activity) to be the most likely—of the targets studied—for any given compound, this approach accurately predicted the documented binding interactions of ABT-737 to Bcl-2, geldanamycin to Hsp90, and fisetin to PI3K (Panel 5c of FIG. 5). On the other hand, nutlin-3a was predicted to bind Bcl-2 with lower affinity than that of its known primary target. MDM2. However, nutlin-3a has also been documented to bind Bcl-2 family proteins, including Bcl-2 and Bcl-XL, suggesting that nutlin-3a may be promiscuous. The docking predictions for BRD-K20733377. BRD-K56819078, and BRD-K44839765 were further investigated, which predicted that all three compounds most likely bind Bcl-2 with binding affinities comparable to those of the geldanamycin-Hsp90 and nutlin-3a-Bcl-2 interactions (Panel 5c of FIG. 5). To further interpret each predicted binding interaction, visualized the docked pose of each compound in comparison to that of ABT-737 was visualized (Panel 5d of FIG. 5). Examination of each binding pose suggested that the three identified compounds could, like ABT-737, interact with residues in common binding pockets of Bcl-2, including those containing L97. A100. G145, and A149 (Panel d of FIG. 5).

The molecular docking simulations suggested that BRD-K20733377. BRD-K56819078, and BRD-K44839765 most likely bind Bcl-2, and that this binding might involve similar residues or binding pockets as those of ABT-737. This hypothesis was directly tested using a time-resolved fluorescence resonance energy transfer (TR-FRET) assay, in which Bcl-2 binding activity to a peptide ligand specific to its active site was measured. It was found that all three compounds indeed inhibited Bcl-2, and this inhibition occurred at micromolar concentrations comparable to the corresponding Snc IC₅₀ values in the therapy-induced and replicative senescence models (Panel e of FIG. 5). Taken together, these results support the hypothesis that BRD-K20733377. BRD-K56819078, and BRD-K44839765 selectively target Snes in part by inhibiting Bcl-2.

### Initial Toxicity Profiling of Identified Compounds

As BRD-K20733377. BRD-K56819078, and BRD-K44839765 exhibit promising senolytic activities and physicochemical properties, their toxicity profiles were further assessed. Mechanistic toxicity, as surveyed by hemolysis, and genotoxicity, as assessed by mutagenic potential, was investigated for the three compounds. Hemolysis is can be a severe toxic liability of systemically-administered compounds that kill cells, and hemolytic activity may preclude the use of compounds for injection. Measuring the release of hemoglobin from human red blood cells extracted from whole blood, it was found that treatment with all three compounds, in addition to ABT-737, did not induce significant hemolysis up to a final concentration of 100 μM—approximately 10× the corresponding therapeutic concentrations in the therapy-induced and replicative senescence models (FIG. 14). In contrast, treatment with Triton X-100, a detergent with hemolytic activity, resulted in substantial hemolysis at concentrations ≥0.01% (w/w). Furthermore, potential genotoxic effects were assessed using a bacterial Ames test, in which the number of bacterial revertants from a base-pair substitution is measured after compound treatment to assess mutagenic potential. In contrast to treatment with ˜1 μM 4-nitroquinoline 1-oxide (4NQO), a potent mutagen, treatment with all three compounds at 100 μM, in addition to ABT-737, did not induce significant reversion of bacterial cultures (FIG. 14). These findings suggest that the identified compounds may possess favorable toxicity profiles.

### In Vivo Efficacy of BRD-K56819078 in an Aged Mouse Model

Given the favorable selectivity and toxicity profiles of BRD-K20733377. BRD-K56819078, and BRD-K44839765. BRD-K56819078, one of the more selective of the three compounds across all senescence models was selected for in vivo testing. Documented measurements of Sncs in animal models have focused on Snc accumulation in the kidneys, which has been suggested to exhibit more salient increases in senescence-associated biomarkers than in other tissues in humans. As a baseline experiment, the kidneys of naïve young and aged C₅₇BL/6J mice were harvested, and SA-β-gal staining and p16 and p21 mRNA expression were measure (Panel 6a of FIG. 6). It was found that the SA-β-gal-positive area in different images of young mouse kidneys were, on average. ˜ 40% less than that of aged mouse kidneys (Panel 6b of FIG. 6). Consistent with a decrease in senescent cell burden, average mRNA expression of p16 and p21 were decreased by >90% and ˜30%, respectively, in young mice relative to aged mice (Panel 6c of FIG. 6). These findings indicate that differences in mouse age, and hence the accumulation of Sncs, are reflected in senescence-associated biomarkers in the kidneys.

BAGedC57BL/6J mice were then treated with vehicle (10% DMSO: 45% PEG300:45% water for injection w/w) or BRD-K56819078 (intraperitoneally at 25 mg/kg per injection) on days 0, 2. 4, 7. 9, and 11 of a 14-day experiment (Panel 6d of FIG. 6). Notably. BRD-K56819078 was well-tolerated by all treated mice and did not result in obvious toxicity, abnormal behavior, or abnormal decreases in weight. The kidneys of all mice were harvested on day 14, and SA-β-gal staining and p16 and p21 mRNA expression were measured. These measurements revealed significant decreases in the SA-β-gal-positive areas of mice treated with BRD-K56819078, with average decreases of ˜20% relative to vehicle-treated mice (Panel 6e of FIG. 6), mRNA expression of p16 and p21 was significantly decreased in mice treated with BRD-K56819078, with average decreases of ˜60% and ˜30%, respectively, in mice treated with BRD-K56819078 relative to vehicle-treated mice (Panel 6f of FIG. 6). Taken together, these in vivo experiments indicate that BRD-K56819078 significantly decreased senescent cell burden and mRNA expression of senescence-associated genes in the kidneys.

### Bcl-XL Targeting of Compounds

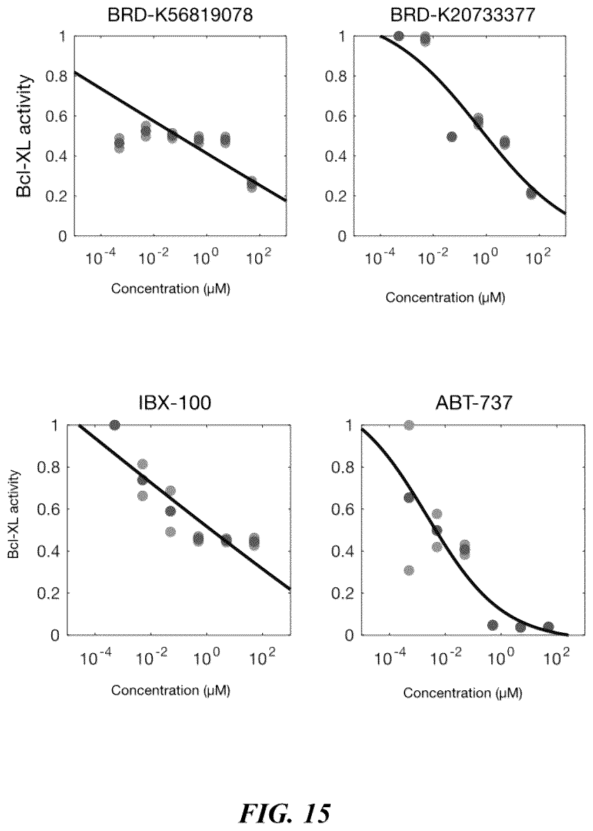

In addition to Bcl-2. IBX-100, and analogs thereof are selective inhibitors of the protein Bcl-XL (IC₅₀ ˜1 μM for all three compounds), without substantively inhibiting the proteins Bcl-w. Bcl2-Al, and Mcl-1 (IC₅₀>100 μM for all three compounds), as demonstrated using TR-FRET experiments (FIG. 15). Our findings appear to indicate that the compounds act similarly to ABT-737 and ABT-263 in terms of primary binding targets.

Although the compounds may share a similar mechanism of action with ABT-737 and ABT-263, it is notable that IBX-100 and analogs are not cytotoxic to T-cells and other immune cells (see Toxicological Properties below). This suggests that the compounds can kill senescent cells without resulting in thrombocytopenia or neutropenia, which has been a challenge in the clinical use of ABT-737 and ABT-263. This may be due to weaker Bcl-XL inhibition than ABT-737 and ABT-263, or the presence of potentially promiscuous and/or potentially toxic chemical moieties in ABT-737 and ABT-263.

### In Vitro Senescent Human Fibroblast Cells

DNA Damage-Induced Senescence

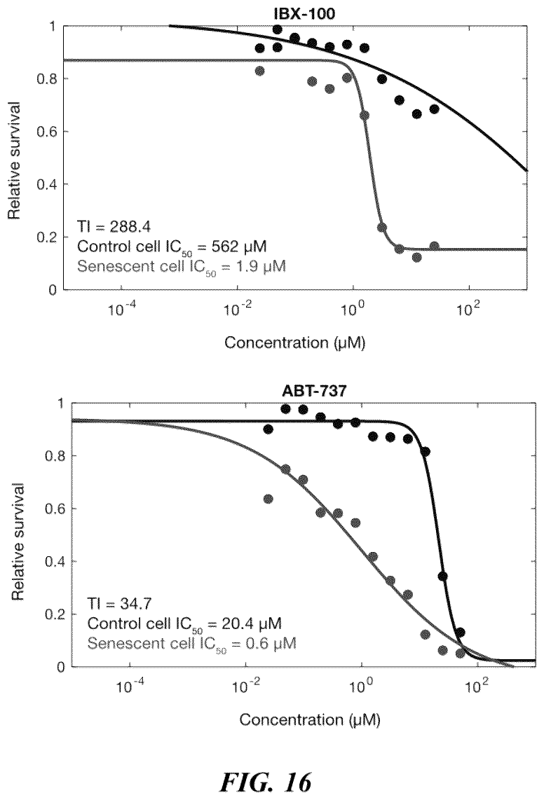

In a model of etoposide-induced senescence in IMR-90 human lung fibroblasts, the compounds disclosed herein including IBX-100 exhibited comparable, if not higher, selectivity against senescent cells than ABT-737. As shown in FIG. 16, the therapeutic index (TI) of IBX-100 was 288.4 while the therapeutic index for ABT-737 was 34.7.

Reactive Oxygen Species-Induced Senescence and Replicative Senescence

Similar results as for DNA damage-induced senescence were found for IMR-90) fibroblasts where senescence was induced by (1) H₂O₂ treatment and (2) continued passaging until cells became non-dividing. In both cases, selectivities were comparable to those in the DNA damage-induced senescence model.

### In Vitro Senescent Human Endothelial Cells and Human Primary Muscle Cells

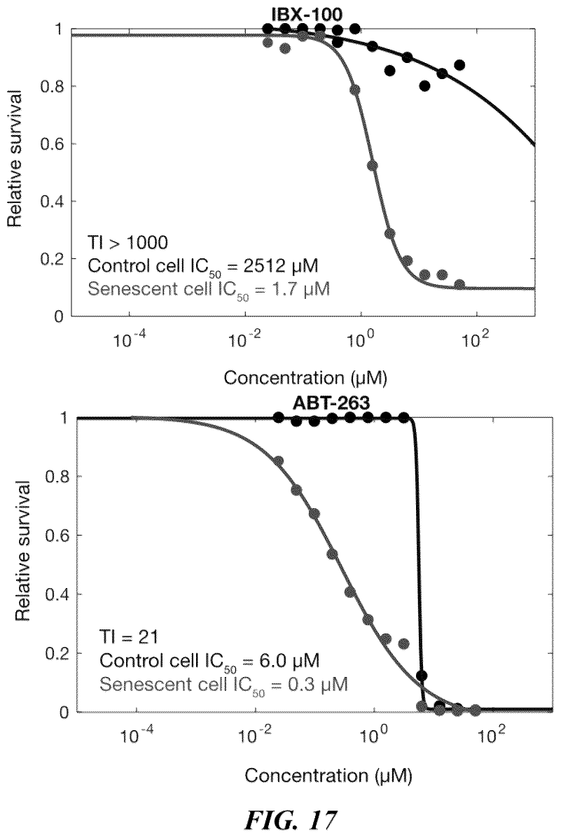

Similar results as for IMR-90 human lung fibroblasts were found in vitro for two diverse cell types, human umbilical vein endothelial cells (HUV-EC-C) and human primary skeletal muscle cells (ATCC PCS-950-010) (FIG. 17), indicating that the senolytic character of the compounds are comparable across different cell types.

### Ex Vivo Aged Human Skin Models

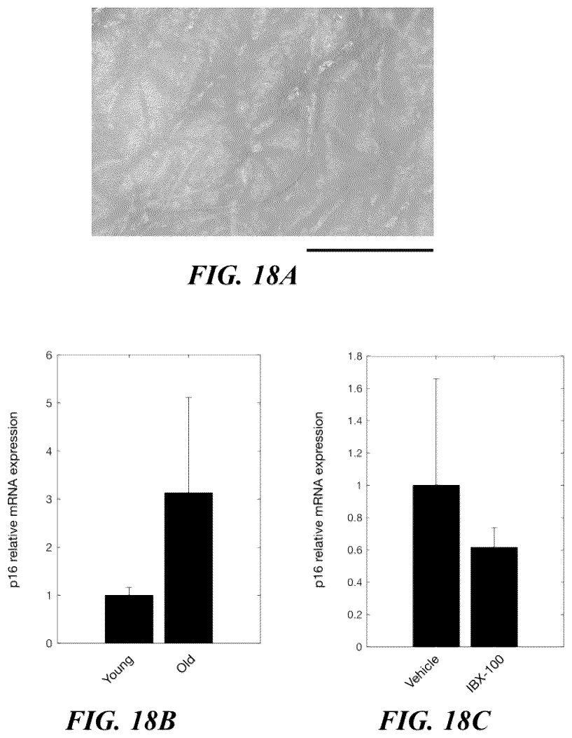

As baseline, mRNA expression of p16 was found to be increased in an older subject (e.g., 70-year-old Caucasian female. Fitzpatrick type 2), by ˜3× compared to a younger subject (e.g., 29-year-old Caucasian female, Fitzpatrick type 3), as shown in FIG. 18.

IBX-100 was first tested in ex vivo skin for safety. IBX-100 was formulated as a 5% topical ointment in 10% DMSO: 45% PEG300:45% water for injection (w/w) and applied directly on an older subject (e.g., 70-year-old Caucasian female, Fitzpatrick type 2) and a younger subject (e.g., 29-year-old Caucasian female, Fitzpatrick type 3) skin. No adverse effects were observed after application.

IBX-100 was further tested in ex vivo skin for efficacy, p16 mRNA expression was measured after 5 days of topical application of IBX-100 on an older subject (e.g., 70-year-old Caucasian female, Fitzpatrick type 2) skin, and compared to vehicle values. We found that p16 mRNA expression decreased, on average, by approximately 40% with administration of IBX-100 for the three samples tested (FIG. 18).

### Ex Vivo Wounded Skin Models

Senescent cells can accumulate at skin wounds and contribute to scarring after wound recovery. Compounds that selectively eliminate senescent cells in wounded skin may facilitate scarless recovery at the cost of longer healing times.

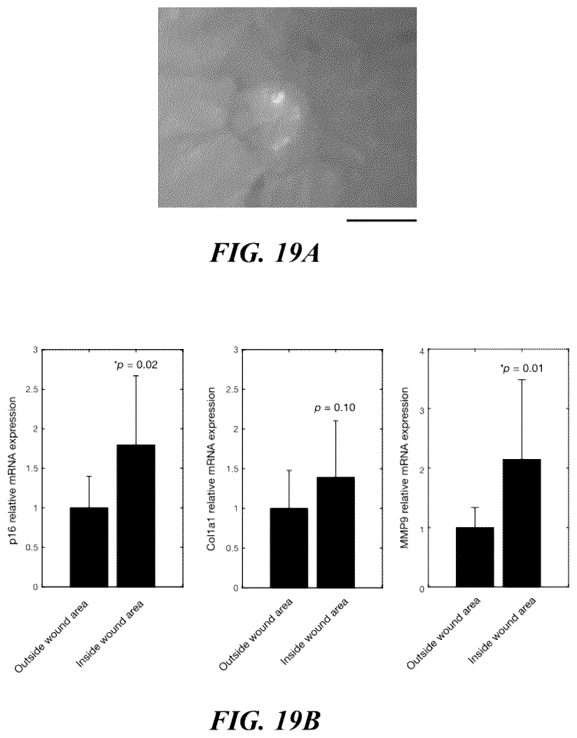

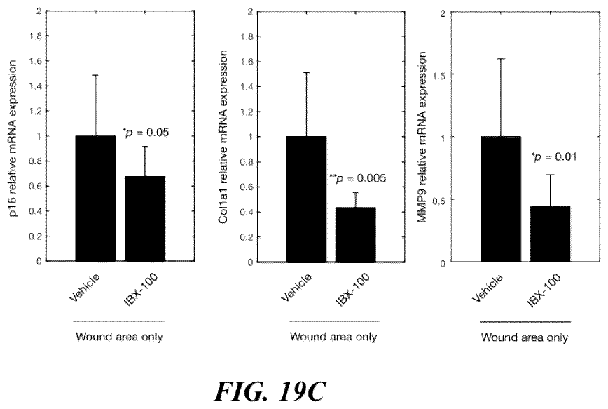

As baseline, skin biopsies were wounded with a 2 mm diameter hole punch, and mRNA expression of p16 was found to be increased in the wound region. This result was comparable for two different sets of skin biopsy samples, including middle-aged skin (e.g., 46-year-old Hispanic female, Fitzpatrick type 3) and younger skin (e.g., 36-year-old Caucasian female, Fitzpatrick type 2-3). For the younger skin sample, RNA expression levels of collagen (Col1a1) and matrix metallopeptidase 9 (MMP9) were additionally measured. Higher levels of Col1a1 may be associated with senescence and scarring. Higher levels of MMP9 may be associated with scarring, arthritis, and inflammation, p16, Col1a1, and MMP9 expression levels were found to be increased, by ˜2×, ˜1.5×, and ˜2×, respectively, inside the wound area as compared to outside the wound area (FIG. 19).

IBX-100 was tested again for safety in a combination dosing regimen. IBX-100 was formulated as a 10% solution in 100% DMSO and applied directly by pipetting 5 μL on wounded younger skin (e.g., 36-year-old Caucasian female, Fitzpatrick type 2-3). Additionally, IBX-100 was administered to the culture medium at a final concentration of about 100 μg/mL, by adding 1 μL of a 100 mg/mL solution in DMSO to 1 mL of culture media, as a model of systemic administration. For both administration routes, no adverse effects were observed after application.

To test efficacy. IBX-100 was applied both topically and systemically as described above every 24 h for 5 days. On day 6, the skin biopsies were excised, and p16. Col1a1, and MMP9 RNA expression was measured inside each wound. Compared to vehicle (e.g., DMSO) values, p16. Col1a1, and MMP9 expression was decreased, on average, by ˜30%. ˜ 60%, and ˜60%, respectively, with administration of IBX-100 (FIG. 19).

### Additional Medicinal Chemistry and Toxicological Properties

Favorable medicinal chemistry properties: IBX-100 and analogs possess more favorable medicinal chemistry properties compared to ABT-737 and ABT-263, with no Lipinski or Veber rule violations. Brenk substructures, and PAINS. The molecular weights of IBX-100 and all analogs are less than 550 Da, suggesting favorable skin permeability.

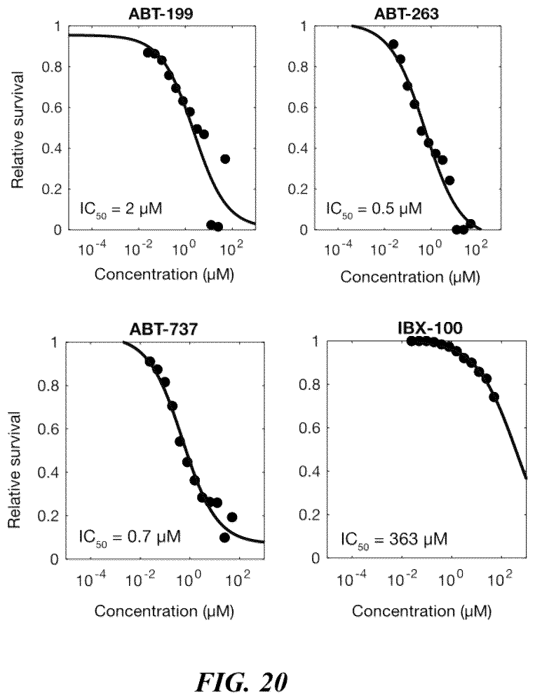

Non-cytotoxicity to T-cells in vitro: IBX-100 appears to be not cytotoxic to primary human T-cells (e.g., IC₅₀)>100 μM for all compounds). In contrast. ABT-737. ABT-263, and ABT-199 may be cytotoxic (e.g., IC₅₀˜1 μM for all three compounds), and induce substantial cell death at about 0.1 μM, as shown in FIG. 20.

### Therapeutic Indices

The N-|2-(2-anilino-2-oxoethyl) sulfanyl-1.3-benzothiazol-6-yl|benzamide core scaffold includes 130 additional compounds (out of 170) tested) for which we have discovered putative selective senolytic activity in vitro in the etoposide-induced senescence model, with therapeutic indices (ratio of IC₅₀) values between etoposide- and DMSO-treated IMR9 ( ) fibroblasts) summarized for each compound (Table 2). Etoposide treatment induces DNA damage, which can result in senescence. DMSO is a vehicle and does not induce DNA damage, which does not result in senescence. Here, selective senolytic activity means inferred therapeutic index greater than 1. Of note, the compounds identified as Specs AN-648/15102282 and Specs AN-648/15102283 are two highly selective senolytic compounds (therapeutic indices >5 to 10). AN-648/15102282 can be described by the IUPAC name N-|2-[2-(4-ethoxyanilino)-2-oxoethyl|sulfanyl-1.3-benzothiazol-6-yl|-3-methylbenzamide. AN-648/15102283 can be described by the IUPAC name N-[2-[2-(4-ethoxyanilino)-2-oxoethyl|sulfanyl-1.3-benzothiazol-6-yl]-2-methylbenzamide. Compounds in Table 2:

BRD-K56819078. BRD-K20733377, and Specs AN-648/15102283 are inhibitors of the protein Bcl-XL (IC₅₀˜1 micromolar for all three compounds), without substantively inhibiting the proteins Bcl-w. Bcl2-Al, and Mcl-1 (IC₅₀ at least approximately 100 μM for all three compounds), as demonstrated using TR-FRET experiments. BRD-K20733377 is additionally an inhibitor of BRD4 (IC₅₀˜10 micromolar).

AN-648/15102283, hereafter referred to as “IBX-100”, decreases p16 mRNA expression when applied topically to old human skin, as well as p16, Col1a1, and MMP9 mRNA expression when applied topically and systemically to wounded human skin. Human skin results are presented for IBX-100. Compounds with a TI of <1 were labelled as +, compounds with a TI of 1 to 5 were labelled as ++, and compounds with a TI of >5 were labeled as +++. In some cases, variation in TIs may occur due biological variation and since the TI is a ratio of two curve-fitted parameters. In some cases, depending on biological variability the TI may vary but will be in the same direction of >1 or <1 (i.e, compounds with +++ or ++ will always be+++ or ++ while compounds with + will always be+).

TABLE 2
Compounds and bioactivity

| cpd | | | Therapeutic |
|---|---|---|---|
| no | Structure | Compound ID | Index (TI) |
| 1 | 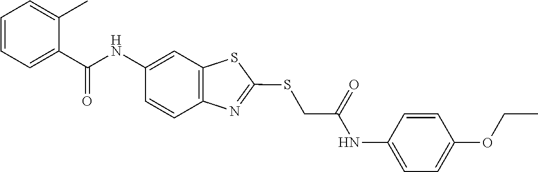 [SMILES: CCOc1ccc(NC(=O)CSc2nc3ccc(NC(=O)c4ccccc4C)cc3s2)cc1] C₂₅H₂₃N₃O₃S₂ | AN- 648/15102283 “IBX-100” | +++ |
| 2 | 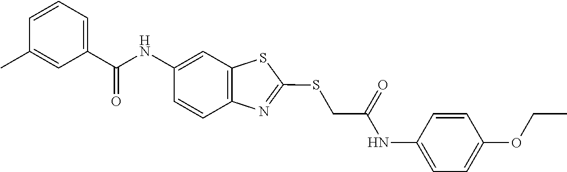 [SMILES: CCOc1ccc(NC(=O)CSc2nc3ccc(NC(=O)c4cccc(C)c4)cc3s2)cc1] C₂₅H₂₃N₃O₃S₂ | AN- 648/15102282 | +++ |
| 3 | 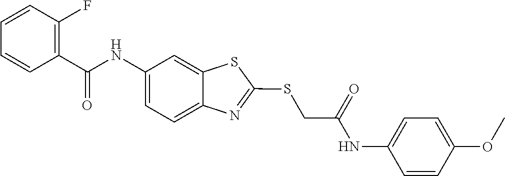 [SMILES: COc1ccc(NC(=O)CSc2nc3ccc(NC(=O)c4ccccc4F)cc3s2)cc1] C₂₃H₁₈FN₃O₃S₂ | STK724162 | +++ |
| 4 | 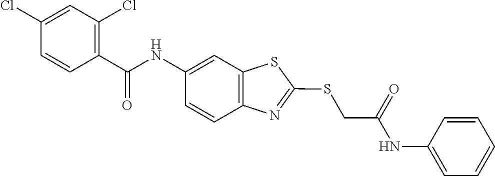 [SMILES: O=C(CSc1nc2ccc(NC(=O)c3ccc(Cl)cc3Cl)cc2s1)Nc1ccccc1] C₂₂H₁₅Cl₂N₃O₂S₂ | AG- 205/34707003 | +++ |
| 5 | 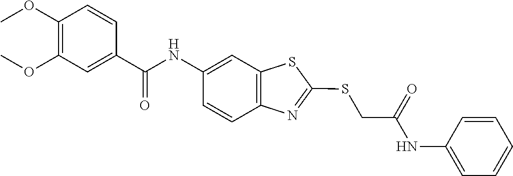 [SMILES: COc1ccc(C(=O)Nc2ccc3nc(SCC(=O)Nc4ccccc4)sc3c2)cc1OC] C₂₄H₂₁N₃O₄S₂ | D336-1892 | +++ |
| 6 | 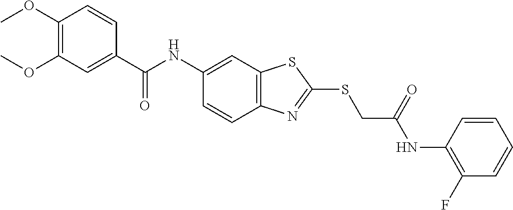 [SMILES: COc1ccc(C(=O)Nc2ccc3nc(SCC(=O)Nc4ccccc4F)sc3c2)cc1OC] C₂₄H₂₀FN₃O₄S₂ | BRD- K56819078/ 7507010 | +++ |
| 7 | 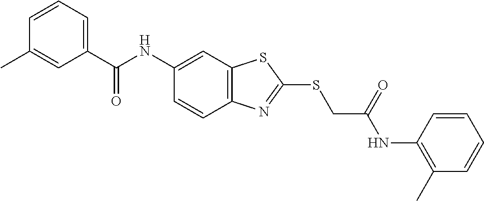 [SMILES: Cc1cccc(C(=O)Nc2ccc3nc(SCC(=O)Nc4ccccc4C)sc3c2)c1] C₂₄H₂₁N₃O₂S₂ | STK232864 | +++ |
| 8 | 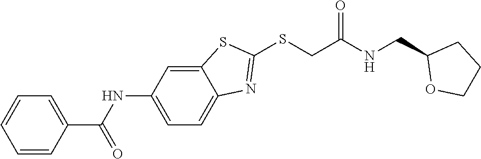 [SMILES: O=C(CSc1nc2ccc(NC(=O)c3ccccc3)cc2s1)NC[C@H]1CCCO1] C₂₁H₂₁N₃O₃S₂ | BRD- A69921102 | +++ |
| 9 | 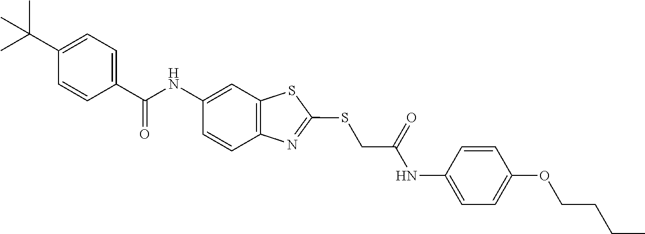 [SMILES: CCCCOc1ccc(NC(=O)CSc2nc3ccc(NC(=O)c4ccc(C(C)(C)C)cc4)cc3s2)cc1] C₃₀N₃O₃S₂ | STK674975 | +++ |
| 10 | 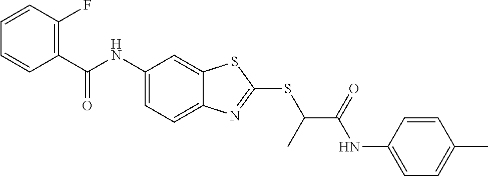 [SMILES: Cc1ccc(NC(=O)C(C)Sc2nc3ccc(NC(=O)c4ccccc4F)cc3s2)cc1] C₂₄H₂₀FN₃O₂S₂ | STK120618 | +++ |
| 11 | 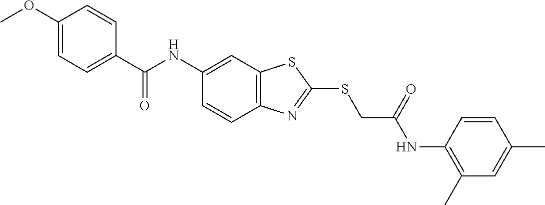 C₂₅H₂₃N₃O₃S₂ | STK130919 | +++ |
| 12 | 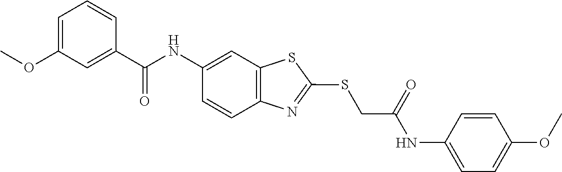 C₂₄H₂₁N₃O₄S₂ | D336-2671 | +++ |
| 13 | 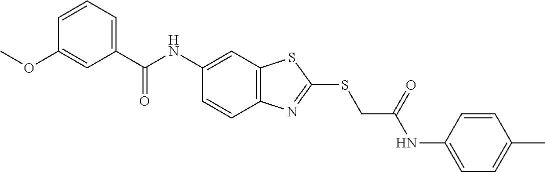 C₂₄H₂₁N₃O₃S₂ | D336-2103 | +++ |
| 14 | 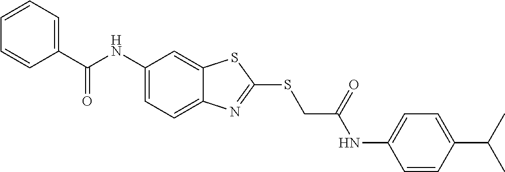 [SMILES: CC(C)c1ccc(NC(=O)CSc2nc3ccc(NC(=O)c4ccccc4)cc3s2)cc1] C₂₅H₂₃N₃O₂S₂ | 6334261 | +++ |
| 15 | 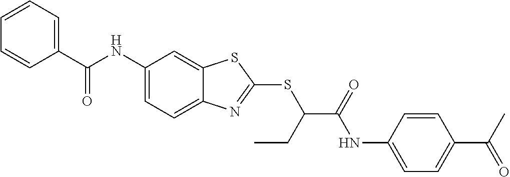 [SMILES: CCC(Sc1nc2ccc(NC(=O)c3ccccc3)cc2s1)C(=O)Nc1ccc(C(C)=O)cc1] C₂₆H₂₃N₃O₃S₂ | STK094888 | +++ |
| 16 | 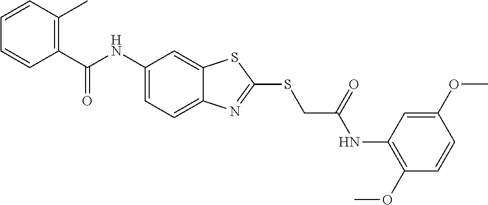 [SMILES: COc1ccc(OC)c(NC(=O)CSc2nc3ccc(NC(=O)c4ccccc4C)cc3s2)c1] C₂₅H₂₃N₃O₄S₂ | STK724172 | +++ |
| 17 | 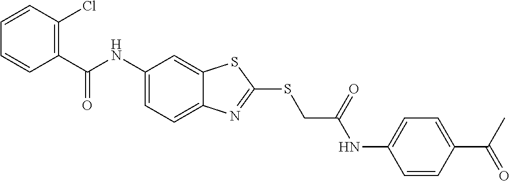 [SMILES: CC(=O)c1ccc(NC(=O)CSc2nc3ccc(NC(=O)c4ccccc4Cl)cc3s2)cc1] C₂₄H₁₈ClN₃O₃S₂ | BRD- K77730668 | +++ |
| 18 | 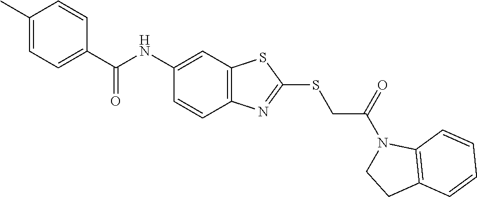 [SMILES: Cc1ccc(C(=O)Nc2ccc3nc(SCC(=O)N4CCc5ccccc54)sc3c2)cc1] C₂₅H₂₁N₃O₂S₂ | D336-0505 | +++ |
| 19 | 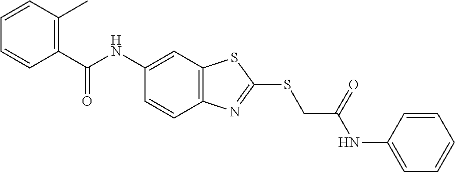 [SMILES: Cc1ccccc1C(=O)Nc1ccc2nc(SCC(=O)Nc3ccccc3)sc2c1] C₂₃H₁₉N₃O₂S₂ | BRD- K44839765 | +++ |
| 20 | 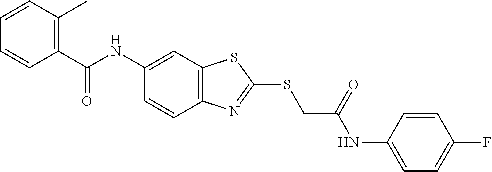 [SMILES: Cc1ccccc1C(=O)Nc1ccc2nc(SCC(=O)Nc3ccc(F)cc3)sc2c1] C₂₃H₁₈FN₃O₂S₂ | BRD- K85007651 | +++ |
| 21 | 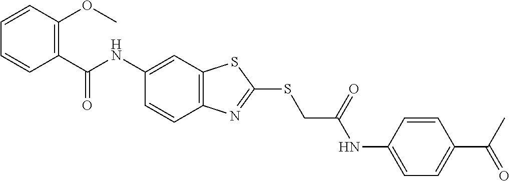 [SMILES: COc1ccccc1C(=O)Nc1ccc2nc(SCC(=O)Nc3ccc(C(C)=O)cc3)sc2c1] C₂₅H₂₁N₃O₄S₂ | STK757643 | +++ |
| 22 | 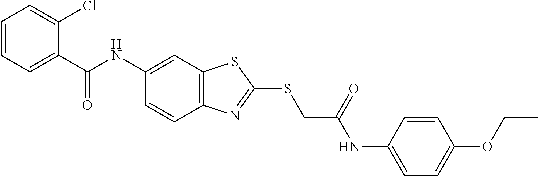 [SMILES: CCOc1ccc(NC(=O)CSc2nc3ccc(NC(=O)c4ccccc4Cl)cc3s2)cc1] C₂₄H₂₀ClN₃O₃S₂ | AN- 648/15102284 | +++ |
| 23 | 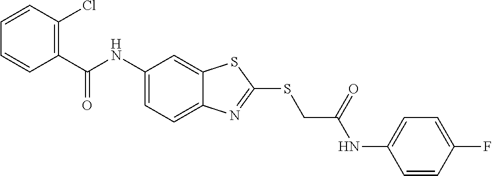 [SMILES: O=C(CSc1nc2ccc(NC(=O)c3ccccc3Cl)cc2s1)Nc1ccc(F)cc1] C₂₂H₁₅ClFN₃O₂S₂ | BRD- K62630232 | +++ |
| 24 | 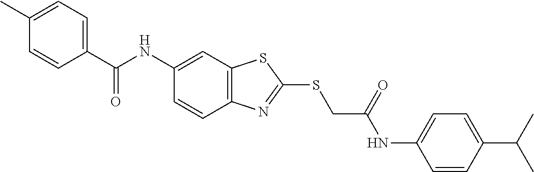 [SMILES: Cc1ccc(C(=O)Nc2ccc3nc(SCC(=O)Nc4ccc(C(C)C)cc4)sc3c2)cc1] C₂₆H₂₅N₃O₂S₂ | AG- 205/12084028 | +++ |
| 25 | 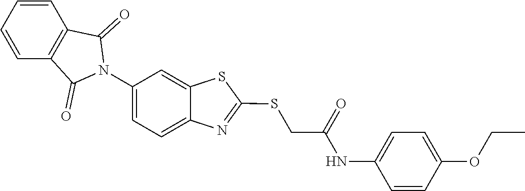 [SMILES: CCOc1ccc(NC(=O)CSc2nc3ccc(N4C(=O)c5ccccc5C4=O)cc3s2)cc1] C₂₅H₁₉N₃O₄S₂ | BRD- K10545557 | +++ |
| 26 | 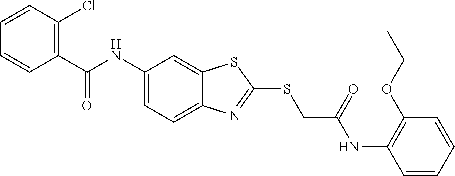 [SMILES: CCOc1ccccc1NC(=O)CSc1nc2ccc(NC(=O)c3ccccc3Cl)cc2s1] C₂₄H₂₀ClN₃O₃S₂ | BRD- K38285873 | +++ |
| 27 | 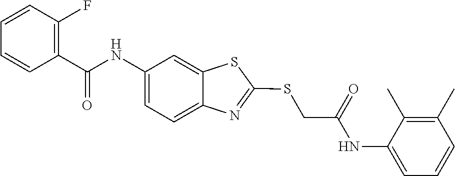 [SMILES: Cc1cccc(NC(=O)CSc2nc3ccc(NC(=O)c4ccccc4F)cc3s2)c1C] C₂₄H₂₀FN₃O₂S₂ | STK724161 | +++ |
| 28 | 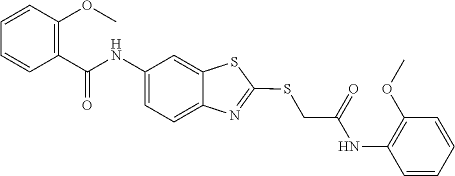 [SMILES: COc1ccccc1NC(=O)CSc1nc2ccc(NC(=O)c3ccccc3OC)cc2s1] C₂₄H₂₁N₃O₄S₂ | BRD- K33858018 | +++ |
| 29 | 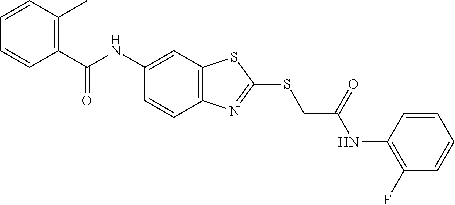 [SMILES: Cc1ccccc1C(=O)Nc1ccc2nc(SCC(=O)Nc3ccccc3F)sc2c1] C₂₃H₁₈FN₃O₂S₂ | 6345429 | ++ |
| 30 | 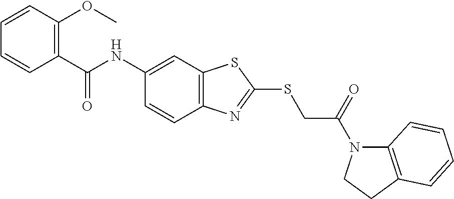 [SMILES: COc1ccccc1C(=O)Nc1ccc2nc(SCC(=O)N3CCc4ccccc43)sc2c1] C₂₅H₂₁N₃O₃S₂ | STK761807 | ++ |
| 31 | 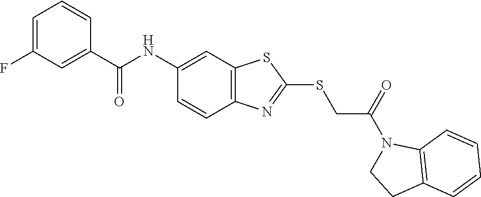 [SMILES: O=C(Nc1ccc2nc(SCC(=O)N3CCc4ccccc43)sc2c1)c1cccc(F)c1] C₂₄H₁₈FN₃O₂S₂ | D336-0549 | ++ |
| 32 | 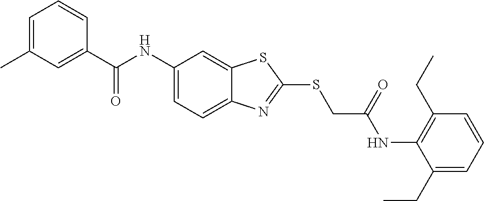 [SMILES: CCc1cccc(CC)c1NC(=O)CSc1nc2ccc(NC(=O)c3cccc(C)c3)cc2s1] C₂₇H₂₇N₃O₂S₂ | STK724282 | ++ |
| 33 | 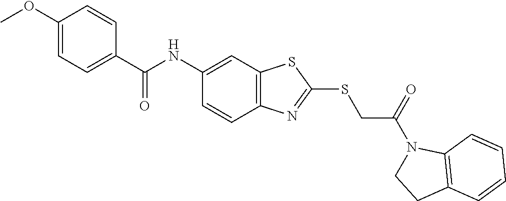 [SMILES: COc1ccc(C(=O)Nc2ccc3nc(SCC(=O)N4CCc5ccccc54)sc3c2)cc1] C₂₅H₂₁N₃O₃S₂ | D336-0539 | ++ |
| 34 | 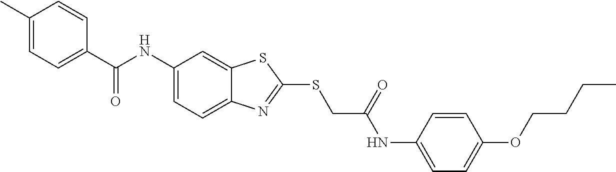 [SMILES: CCCCOc1ccc(NC(=O)CSc2nc3ccc(NC(=O)c4ccc(C)cc4)cc3s2)cc1] C₂₇N₃O₃S₂ | STK759017 | ++ |
| 35 | 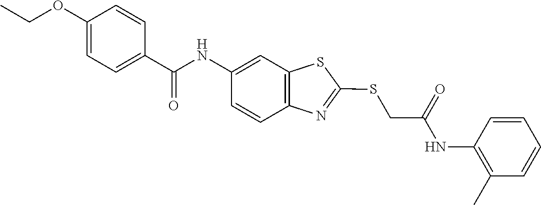 [SMILES: CCOc1ccc(C(=O)Nc2ccc3nc(SCC(=O)Nc4ccccc4C)sc3c2)cc1] C₂₅H₂₃N₃O₃S₂ | D336-1974 | ++ |
| 36 | 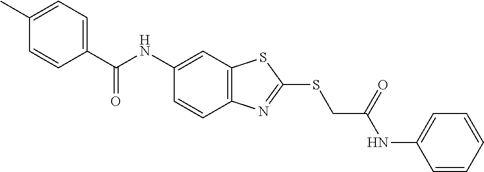 [SMILES: Cc1ccc(C(=O)Nc2ccc3nc(SCC(=O)Nc4ccccc4)sc3c2)cc1] C₂₃H₁₉N₃O₂S₂ | AG- 205/34707044 | ++ |
| 37 | 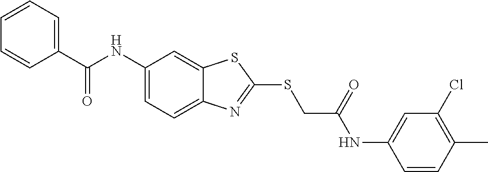 [SMILES: Cc1ccc(NC(=O)CSc2nc3ccc(NC(=O)c4ccccc4)cc3s2)cc1Cl] C₂₃H₁₈ClN₃O₂S₂ | STK724139 | ++ |
| 38 | 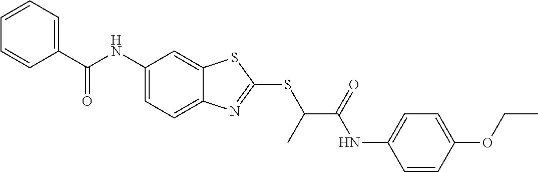 [SMILES: CCOc1ccc(NC(=O)C(C)Sc2nc3ccc(NC(=O)c4ccccc4)cc3s2)cc1] C₂₅H₂₃N₃O₃S₂ | STK868732 | ++ |
| 39 | 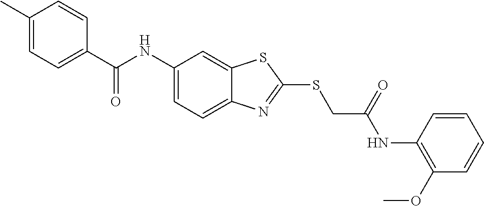 [SMILES: COc1ccccc1NC(=O)CSc1nc2ccc(NC(=O)c3ccc(C)cc3)cc2s1] C₂₄H₂₁N₃O₃S₂ | 6006204 | ++ |
| 40 | 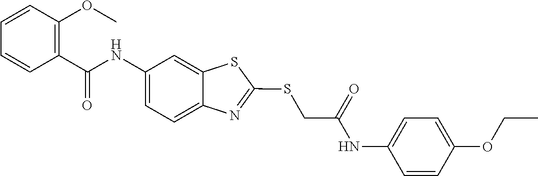 [SMILES: CCOc1ccc(NC(=O)CSc2nc3ccc(NC(=O)c4ccccc4OC)cc3s2)cc1] C₂₅H₂₃N₃O₄S₂ | 6320882 | ++ |
| 41 | 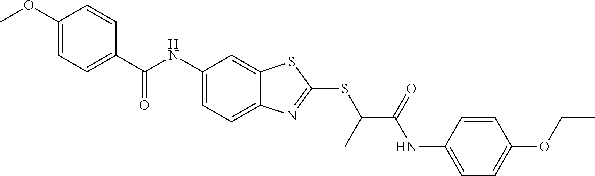 [SMILES: CCOc1ccc(NC(=O)C(C)Sc2nc3ccc(NC(=O)c4ccc(OC)cc4)cc3s2)cc1] C₂₆N₃O₄S₂ | STK130497 | ++ |
| 42 | 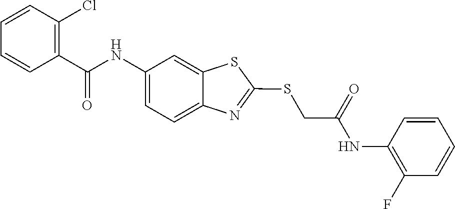 [SMILES: O=C(CSc1nc2ccc(NC(=O)c3ccccc3Cl)cc2s1)Nc1ccccc1F] C₂₂H₁₅ClFN₃O₂S₂ | 6353965 | ++ |
| 43 | 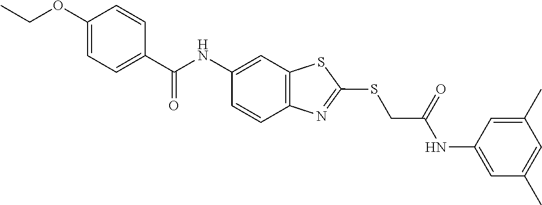 [SMILES: CCOc1ccc(C(=O)Nc2ccc3nc(SCC(=O)Nc4cc(C)cc(C)c4)sc3c2)cc1] C₂₆H₂₅N₃O₃S₂ | D336-2897 | ++ |
| 44 | 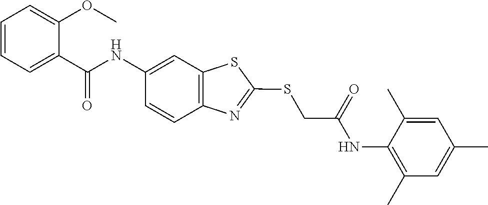 [SMILES: COc1ccccc1C(=O)Nc1ccc2nc(SCC(=O)Nc3c(C)cc(C)cc3C)sc2c1] C₂₆H₂₅N₃O₃S₂ | AG- 205/12084161 | ++ |
| 45 | 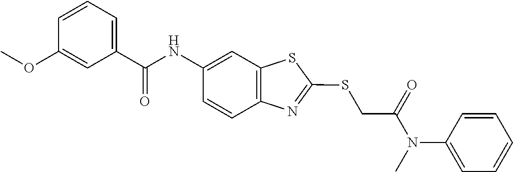 [SMILES: COc1cccc(C(=O)Nc2ccc3nc(SCC(=O)N(C)c4ccccc4)sc3c2)c1] C₂₄H₂₁N₃O₃S₂ | D336-1180 | ++ |
| 46 | 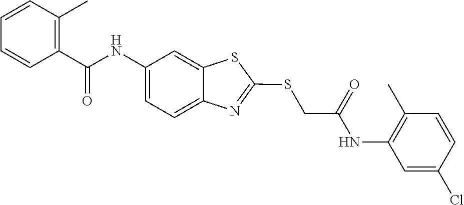 [SMILES: Cc1ccc(Cl)cc1NC(=O)CSc1nc2ccc(NC(=O)c3ccccc3C)cc2s1] C₂₄H₂₀ClN₃O₂S₂ | STK724173 | ++ |
| 47 | 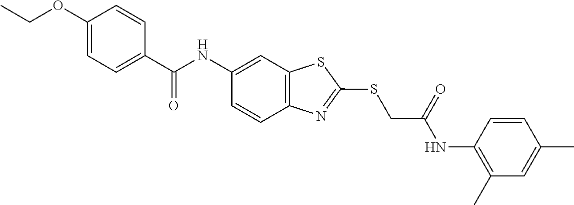 [SMILES: CCOc1ccc(C(=O)Nc2ccc3nc(SCC(=O)Nc4ccc(C)cc4C)sc3c2)cc1] C₂₆H₂₅N₃O₃S₂ | D336-2755 | ++ |
| 48 | 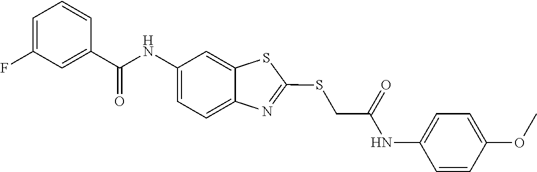 [SMILES: COc1ccc(NC(=O)CSc2nc3ccc(NC(=O)c4cccc(F)c4)cc3s2)cc1] C₂₃H₁₈FN₃O₃S₂ | D336-2679 | ++ |
| 49 | 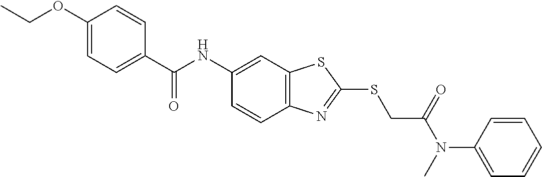 [SMILES: CCOc1ccc(C(=O)Nc2ccc3nc(SCC(=O)N(C)c4ccccc4)sc3c2)cc1] C₂₅H₂₃N₃O₃S₂ | D336-1193 | ++ |
| 50 | 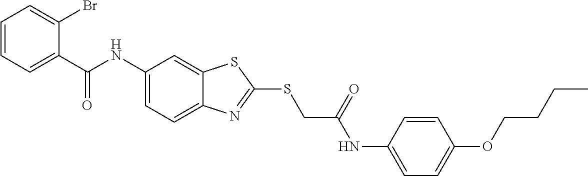 [SMILES: CCCCOc1ccc(NC(=O)CSc2nc3ccc(NC(=O)c4ccccc4Br)cc3s2)cc1] C₂₆BrN₃O₃S₂ | STK674366 | ++ |
| 51 | 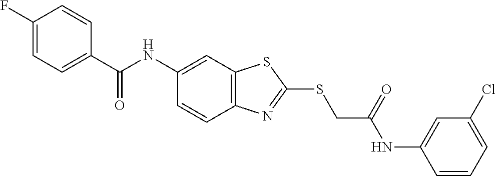 [SMILES: O=C(CSc1nc2ccc(NC(=O)c3ccc(F)cc3)cc2s1)Nc1cccc(Cl)c1] | AQ- 088/42014162 | ++ |
| 52 | 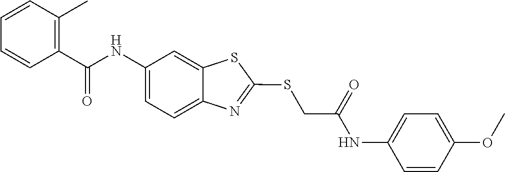 [SMILES: COc1ccc(NC(=O)CSc2nc3ccc(NC(=O)c4ccccc4C)cc3s2)cc1] C₂₄H₂₁N₃O₃S₂ | D336-2650 | ++ |
| 53 | 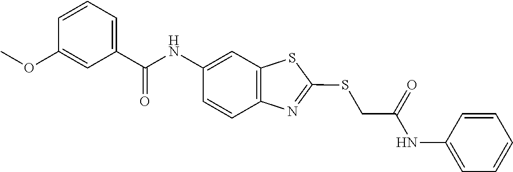 [SMILES: COc1cccc(C(=O)Nc2ccc3nc(SCC(=O)Nc4ccccc4)sc3c2)c1] C₂₃H₁₉N₃O₃S₂ | D336-1890 | ++ |
| 54 | 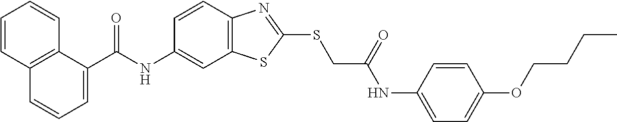 [SMILES: CCCCOc1ccc(NC(=O)CSc2nc3ccc(NC(=O)c4cccc5ccccc45)cc3s2)cc1] C₃₀N₃O₃S₂ | STK675416 | ++ |
| 55 | 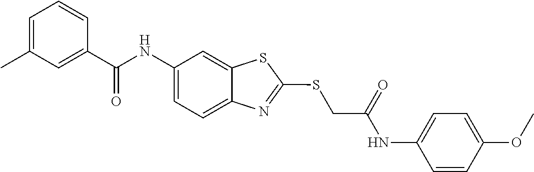 C₂₄H₂₁N₃O₃S₂ | D336-2651 | ++ |
| 56 | 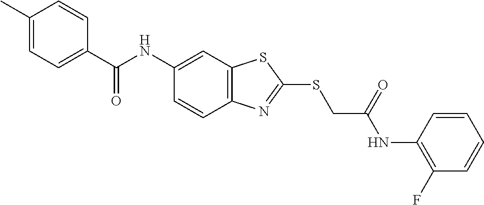 [SMILES: Cc1ccc(C(=O)Nc2ccc3nc(SCC(=O)Nc4ccccc4F)sc3c2)cc1] C₂₃H₁₈FN₃O₂S₂ | STK724184 | ++ |
| 57 | 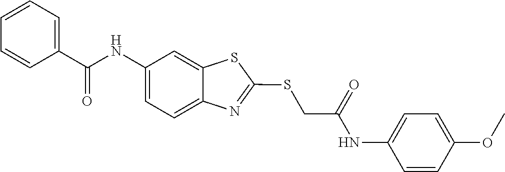 [SMILES: COc1ccc(NC(=O)CSc2nc3ccc(NC(=O)c4ccccc4)cc3s2)cc1] C₂₃H₁₉N₃O₃S₂ | 3167-1078 | ++ |
| 58 | 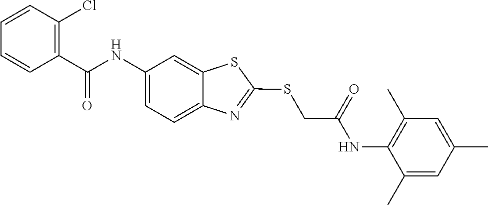 [SMILES: Cc1cc(C)c(NC(=O)CSc2nc3ccc(NC(=O)c4ccccc4Cl)cc3s2)c(C)c1] C₂₅H₂₂ClN₃O₂S₂ | BRD- K63886919 | ++ |
| 59 | 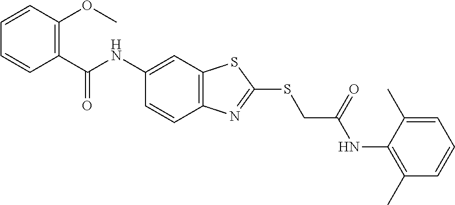 [SMILES: COc1ccccc1C(=O)Nc1ccc2nc(SCC(=O)Nc3c(C)cccc3C)sc2c1] C₂₅H₂₃N₃O₃S₂ | STK759009 | ++ |
| 60 | 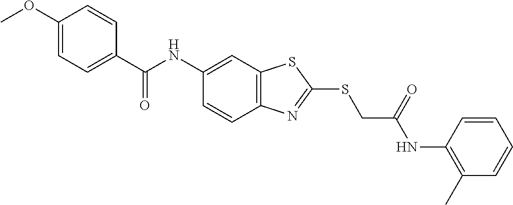 [SMILES: COc1ccc(C(=O)Nc2ccc3nc(SCC(=O)Nc4ccccc4C)sc3c2)cc1] C₂₄H₂₁N₃O₃S₂ | D336-1959 | ++ |
| 61 | 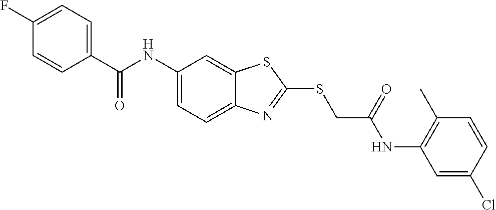 [SMILES: Cc1ccc(Cl)cc1NC(=O)CSc1nc2ccc(NC(=O)c3ccc(F)cc3)cc2s1] C₂₃H₁₇ClFN₃O₂S₂ | STK138751 | ++ |
| 62 | 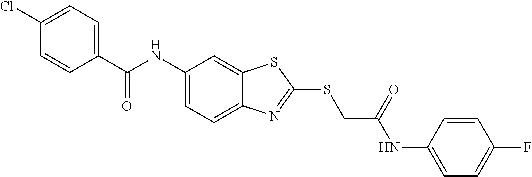 [SMILES: O=C(CSc1nc2ccc(NC(=O)c3ccc(Cl)cc3)cc2s1)Nc1ccc(F)cc1] C₂₂H₁₅ClFN₃O₂S₂ | 6317712 | ++ |
| 63 | 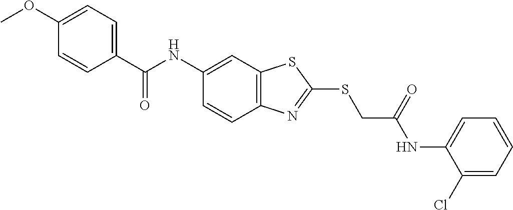 [SMILES: COc1ccc(C(=O)Nc2ccc3nc(SCC(=O)Nc4ccccc4Cl)sc3c2)cc1] C₂₃H₁₈ClN₃O₃S₂ | AQ- 088/42181469 | ++ |
| 64 | 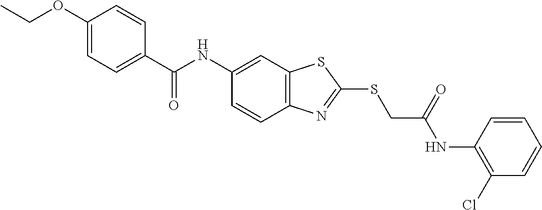 [SMILES: CCOc1ccc(C(=O)Nc2ccc3nc(SCC(=O)Nc4ccccc4Cl)sc3c2)cc1] C₂₄H₂₀ClN₃O₃S₂ | D336-2329 | ++ |
| 65 | 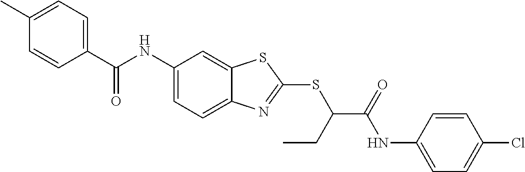 [SMILES: CCC(Sc1nc2ccc(NC(=O)c3ccc(C)cc3)cc2s1)C(=O)Nc1ccc(Cl)cc1] C₂₅H₂₂ClN₃O₂S₂ | STK030991 | ++ |
| 66 | 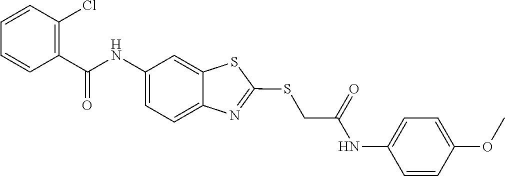 [SMILES: COc1ccc(NC(=O)CSc2nc3ccc(NC(=O)c4ccccc4Cl)cc3s2)cc1] C₂₃H₁₈ClN₃O₃S₂ | D336-2636 | ++ |
| 67 | 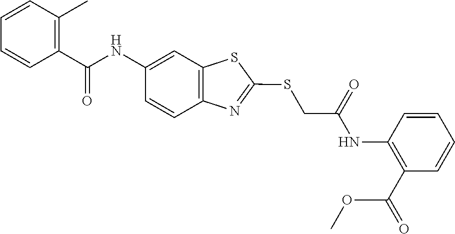 [SMILES: COC(=O)c1ccccc1NC(=O)CSc1nc2ccc(NC(=O)c3ccccc3C)cc2s1] C₂₅H₂₁N₃O₄S₂ | 6002181 | ++ |
| 68 | 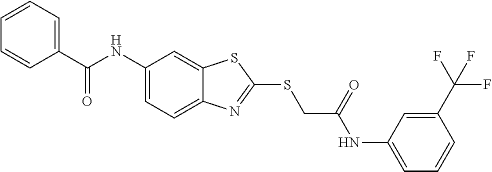 [SMILES: O=C(CSc1nc2ccc(NC(=O)c3ccccc3)cc2s1)Nc1cccc(C(F)(F)F)c1] C₂₃H₁₆F₃N₃O₂S₂ | 7248099 | ++ |
| 69 | 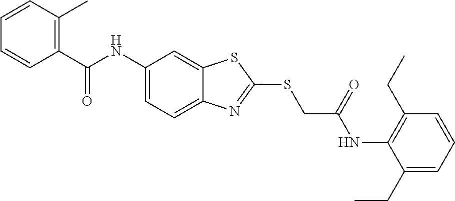 [SMILES: CCc1cccc(CC)c1NC(=O)CSc1nc2ccc(NC(=O)c3ccccc3C)cc2s1] C₂₇H₂₇N₃O₂S₂ | STK724171 | ++ |
| 70 | 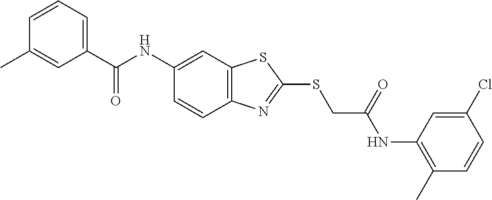 [SMILES: Cc1cccc(C(=O)Nc2ccc3nc(SCC(=O)Nc4cc(Cl)ccc4C)sc3c2)c1] C₂₄H₂₀ClN₃O₂S₂ | STK724285 | ++ |
| 71 | 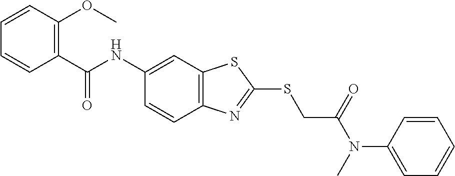 [SMILES: COc1ccccc1C(=O)Nc1ccc2nc(SCC(=O)N(C)c3ccccc3)sc2c1] C₂₄H₂₁N₃O₃S₂ | D336-1177 | ++ |
| 72 | 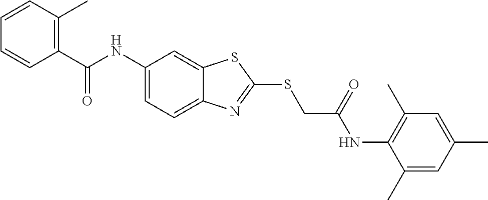 [SMILES: Cc1cc(C)c(NC(=O)CSc2nc3ccc(NC(=O)c4ccccc4C)cc3s2)c(C)c1] C₂₆H₂₅N₃O₂S₂ | BRD- K63078176 | ++ |
| 73 | 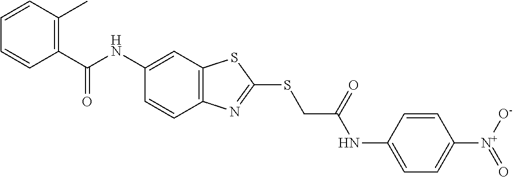 [SMILES: Cc1ccccc1C(=O)Nc1ccc2nc(SCC(=O)Nc3ccc([N+](=O)[O-])cc3)sc2c1] C₂₃H₁₈N₄O₄S₂ | BRD- K25439112 | ++ |
| 74 | 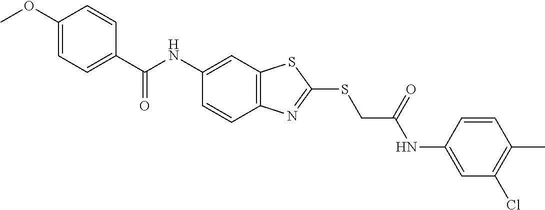 C₂₄H₂₀ClN₃O₃S₂ | AQ- 088/42013422 | ++ |
| 75 | 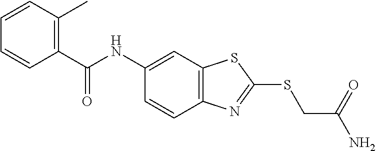 [SMILES: Cc1ccccc1C(=O)Nc1ccc2nc(SCC(N)=O)sc2c1] C₁₇H₁₅N₃O₂S₂ | D336-6058 | ++ |
| 76 | 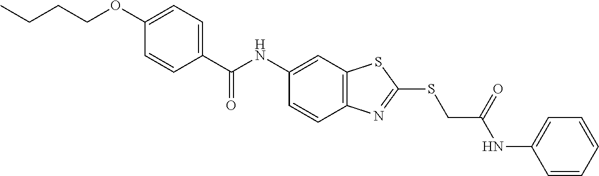 [SMILES: CCCCOc1ccc(C(=O)Nc2ccc3nc(SCC(=O)Nc4ccccc4)sc3c2)cc1] C₂₆H₂₅N₃O₃S₂ | AN- 648/15102250 | ++ |
| 77 | 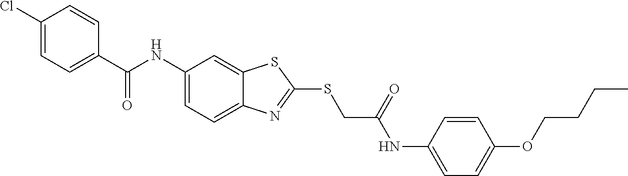 [SMILES: CCCCOc1ccc(NC(=O)CSc2nc3ccc(NC(=O)c4ccc(Cl)cc4)cc3s2)cc1] C₂₆ClN₃O₃S₂ | STK675550 | ++ |
| 78 | 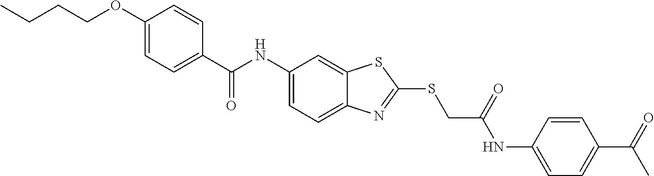 [SMILES: CCCCOc1ccc(C(=O)Nc2ccc3nc(SCC(=O)Nc4ccc(C(C)=O)cc4)sc3c2)cc1] C₂₈N₃O₄S₂ | STK673515 | ++ |
| 79 | 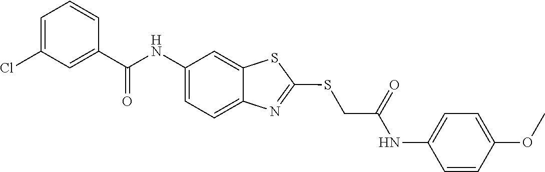 [SMILES: COc1ccc(NC(=O)CSc2nc3ccc(NC(=O)c4cccc(Cl)c4)cc3s2)cc1] C₂₃H₁₈ClN₃O₃S₂ | D336-2676 | ++ |
| 80 | 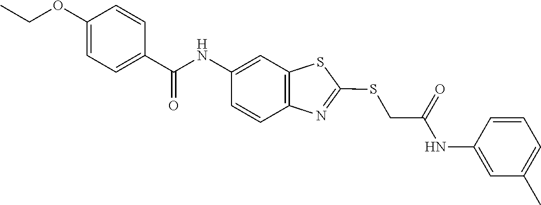 [SMILES: CCOc1ccc(C(=O)Nc2ccc3nc(SCC(=O)Nc4cccc(C)c4)sc3c2)cc1] C₂₅H₂₃N₃O₃S₂ | D336-2045 | ++ |
| 81 | 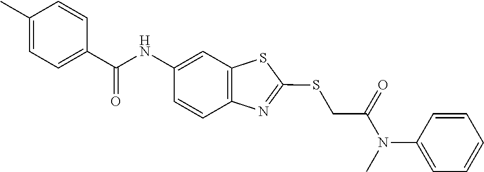 [SMILES: Cc1ccc(C(=O)Nc2ccc3nc(SCC(=O)N(C)c4ccccc4)sc3c2)cc1] C₂₄H₂₁N₃O₂S₂ | D336-1144 | ++ |
| 82 | 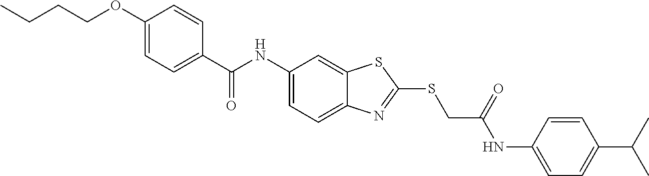 [SMILES: CCCCOc1ccc(C(=O)Nc2ccc3nc(SCC(=O)Nc4ccc(C(C)C)cc4)sc3c2)cc1] C₂₉N₃O₃S₂ | STK674663 | ++ |
| 83 | 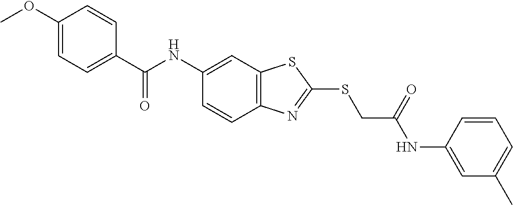 C₂₄H₂₁N₃O₃S₂ | D336-2030 | ++ |
| 84 | 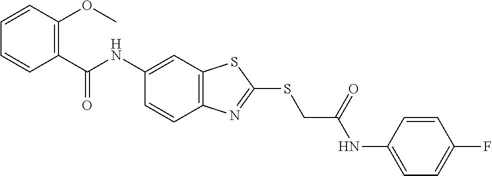 [SMILES: COc1ccccc1C(=O)Nc1ccc2nc(SCC(=O)Nc3ccc(F)cc3)sc2c1] C₂₃H₁₈FN₃O₃S₂ | STK757641 | ++ |
| 85 | 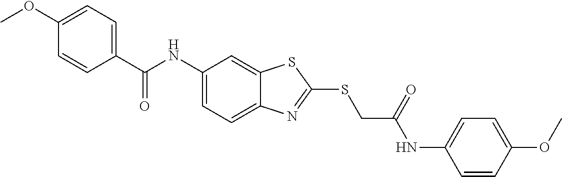 C₂₄H₂₁N₃O₄S₂ | D336-2669 | ++ |
| 86 | 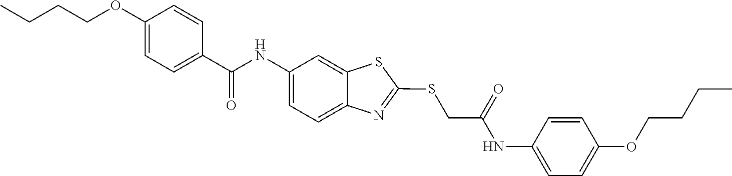 [SMILES: CCCCOc1ccc(NC(=O)CSc2nc3ccc(NC(=O)c4ccc(OCCCC)cc4)cc3s2)cc1] C₃₀N₃O₄S₂ | STK674751 | ++ |
| 87 | 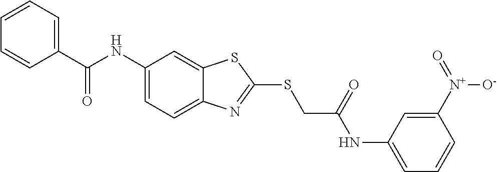 [SMILES: O=C(CSc1nc2ccc(NC(=O)c3ccccc3)cc2s1)Nc1cccc([N+](=O)[O-])c1] C₂₂H₁₆N₄O₄S₂ | BRD- K54301655 | ++ |
| 88 | 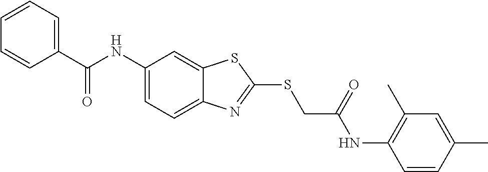 [SMILES: Cc1ccc(NC(=O)CSc2nc3ccc(NC(=O)c4ccccc4)cc3s2)c(C)c1] C₂₄H₂₁N₃O₂S₂ | 6314424 | ++ |
| 89 | 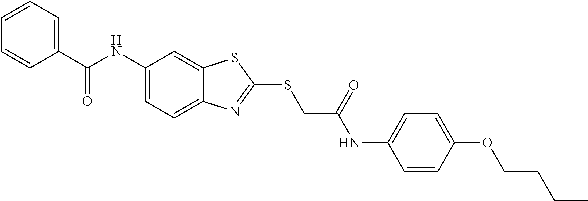 [SMILES: CCCCOc1ccc(NC(=O)CSc2nc3ccc(NC(=O)c4ccccc4)cc3s2)cc1] C₂₆H₂₅N₃O₃S₂ | STK758036 | ++ |
| 90 | 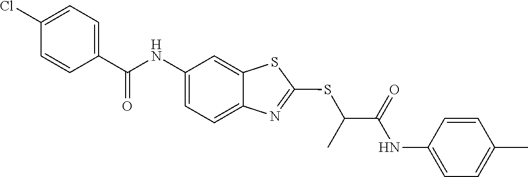 [SMILES: Cc1ccc(NC(=O)C(C)Sc2nc3ccc(NC(=O)c4ccc(Cl)cc4)cc3s2)cc1] C₂₄H₂₀ClN₃O₂S₂ | STK131424 | ++ |
| 91 | 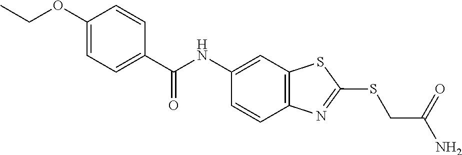 [SMILES: CCOc1ccc(C(=O)Nc2ccc3nc(SCC(N)=O)sc3c2)cc1] C₁₈H₁₇N₃O₃S₂ | D336-6092 | ++ |
| 92 | 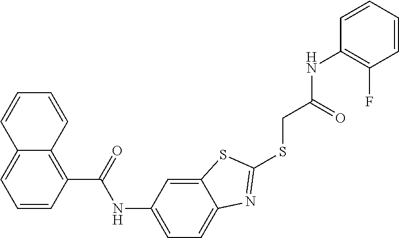 [SMILES: O=C(CSc1nc2ccc(NC(=O)c3cccc4ccccc34)cc2s1)Nc1ccccc1F] C₂₆H₁₈FN₃O₂S₂ | STK759081 | ++ |
| 93 | 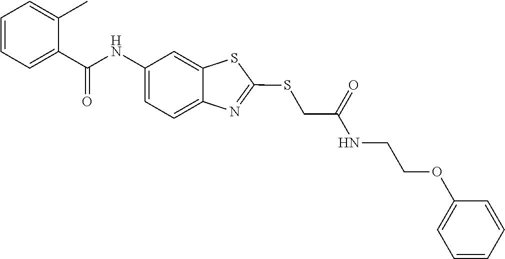 [SMILES: Cc1ccccc1C(=O)Nc1ccc2nc(SCC(=O)NCCOc3ccccc3)sc2c1] C₂₅N₃O₃S₂ | STK724169 | ++ |
| 94 | 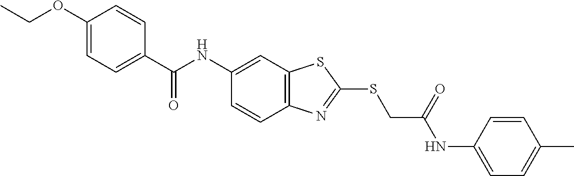 [SMILES: CCOc1ccc(C(=O)Nc2ccc3nc(SCC(=O)Nc4ccc(C)cc4)sc3c2)cc1] C₂₅H₂₃N₃O₃S₂ | D336-2116 | ++ |
| 95 | 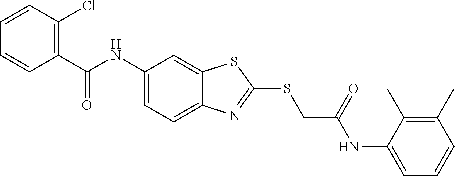 [SMILES: Cc1cccc(NC(=O)CSc2nc3ccc(NC(=O)c4ccccc4Cl)cc3s2)c1C] C₂₄H₂₀ClN₃O₂S₂ | 6331264 | ++ |
| 96 | 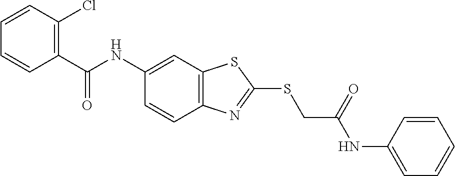 [SMILES: O=C(CSc1nc2ccc(NC(=O)c3ccccc3Cl)cc2s1)Nc1ccccc1] C₂₂H₁₆ClN₃O₂S₂ | STK724148 | ++ |
| 97 | 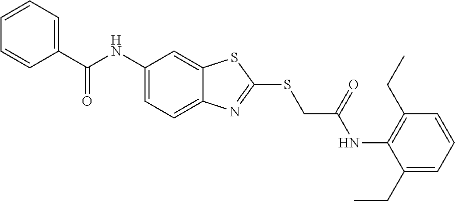 [SMILES: CCc1cccc(CC)c1NC(=O)CSc1nc2ccc(NC(=O)c3ccccc3)cc2s1] C₂₆H₂₅N₃O₂S₂ | STK724136 | ++ |
| 98 | 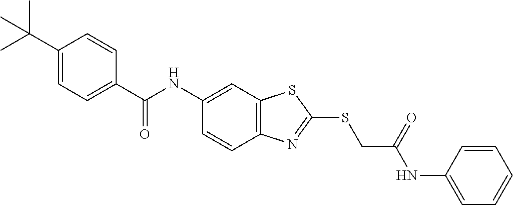 [SMILES: CC(C)(C)c1ccc(C(=O)Nc2ccc3nc(SCC(=O)Nc4ccccc4)sc3c2)cc1] C₂₆H₂₅N₃O₂S₂ | STK724196 | ++ |
| 99 | 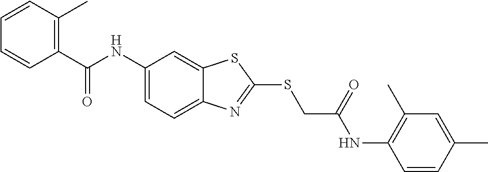 [SMILES: Cc1ccc(NC(=O)CSc2nc3ccc(NC(=O)c4ccccc4C)cc3s2)c(C)c1] C₂₅H₂₃N₃O₂S₂ | D336-2721 | ++ |
| 100 | 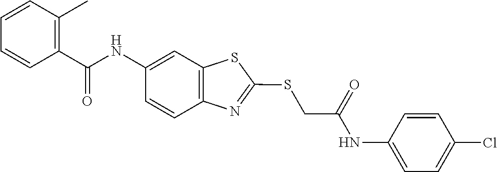 [SMILES: Cc1ccccc1C(=O)Nc1ccc2nc(SCC(=O)Nc3ccc(Cl)cc3)sc2c1] C₂₃H₁₈ClN₃O₂S₂ | D336-2366 | ++ |
| 101 | 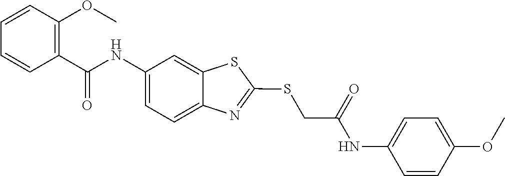 [SMILES: COc1ccc(NC(=O)CSc2nc3ccc(NC(=O)c4ccccc4OC)cc3s2)cc1] C₂₄H₂₁N₃O₄S₂ | D336-2668 | ++ |
| 102 | 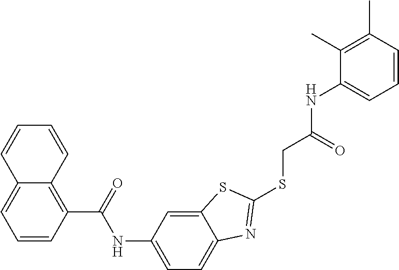 [SMILES: Cc1cccc(NC(=O)CSc2nc3ccc(NC(=O)c4cccc5ccccc45)cc3s2)c1C] C₂₈H₂₃N₃O₂S₂ | STK760822 | ++ |
| 103 | 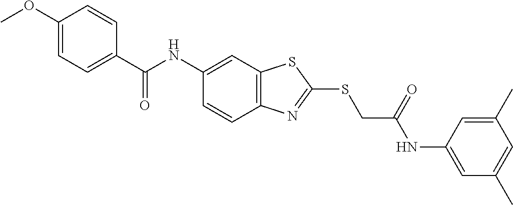 C₂₅H₂₃N₃O₃S₂ | D336-2882 | ++ |
| 104 | 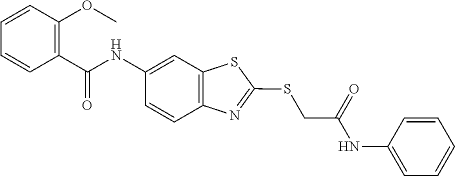 [SMILES: COc1ccccc1C(=O)Nc1ccc2nc(SCC(=O)Nc3ccccc3)sc2c1] C₂₃H₁₉N₃O₃S₂ | 8008-2380 | ++ |
| 105 | 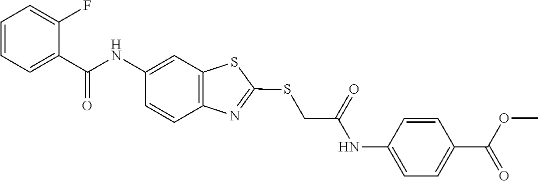 [SMILES: COC(=O)c1ccc(NC(=O)CSc2nc3ccc(NC(=O)c4ccccc4F)cc3s2)cc1] C₂₄H₁₈FN₃O₄S₂ | AM- 879/14775331 | ++ |
| 106 | 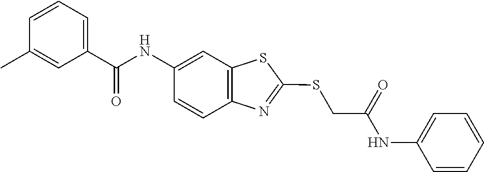 [SMILES: Cc1cccc(C(=O)Nc2ccc3nc(SCC(=O)Nc4ccccc4)sc3c2)c1] C₂₃H₁₉N₃O₂S₂ | AN- 648/15101245 | ++ |
| 107 | 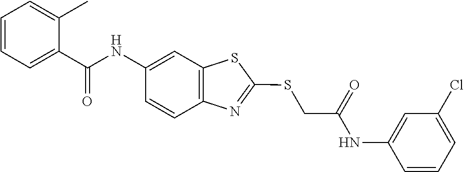 [SMILES: Cc1ccccc1C(=O)Nc1ccc2nc(SCC(=O)Nc3cccc(Cl)c3)sc2c1] | AQ- 088/42014092 | ++ |
| 108 | 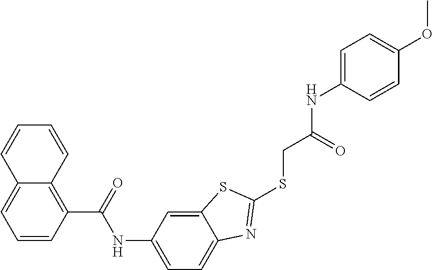 [SMILES: COc1ccc(NC(=O)CSc2nc3ccc(NC(=O)c4cccc5ccccc45)cc3s2)cc1] C₂₇H₂₁N₃O₃S₂ | STK760058 | ++ |
| 109 | 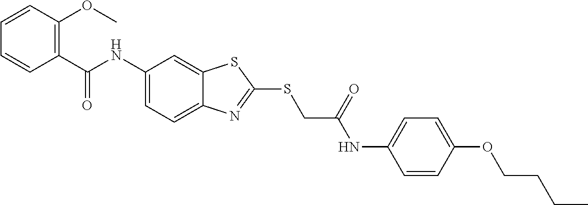 [SMILES: CCCCOc1ccc(NC(=O)CSc2nc3ccc(NC(=O)c4ccccc4OC)cc3s2)cc1] C₂₇H₂₇N₃O₄S₂ | 8008-5808 | ++ |
| 110 | 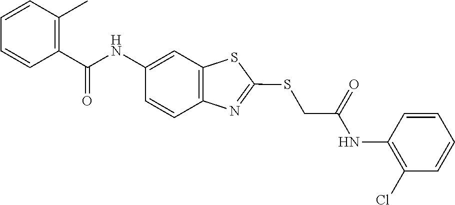 [SMILES: Cc1ccccc1C(=O)Nc1ccc2nc(SCC(=O)Nc3ccccc3Cl)sc2c1] C₂₃H₁₈ClN₃O₂S₂ | D336-2295 | ++ |
| 111 | 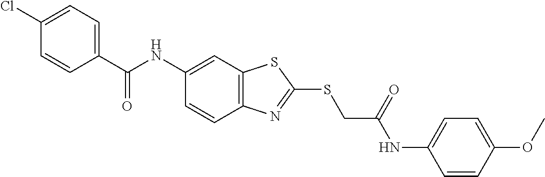 [SMILES: COc1ccc(NC(=O)CSc2nc3ccc(NC(=O)c4ccc(Cl)cc4)cc3s2)cc1] C₂₃H₁₈ClN₃O₃S₂ | D336-2631 | ++ |
| 112 | 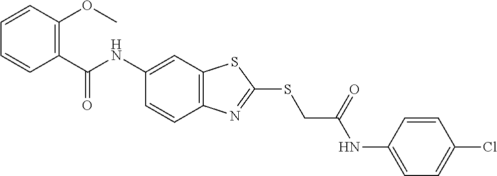 [SMILES: COc1ccccc1C(=O)Nc1ccc2nc(SCC(=O)Nc3ccc(Cl)cc3)sc2c1] C₂₃H₁₈ClN₃O₃S₂ | D336-2384 | ++ |
| 113 | 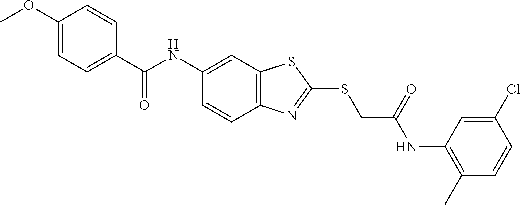 [SMILES: COc1ccc(C(=O)Nc2ccc3nc(SCC(=O)Nc4cc(Cl)ccc4C)sc3c2)cc1] C₂₄H₂₀ClN₃O₃S₂ | STK129943 | ++ |
| 114 | 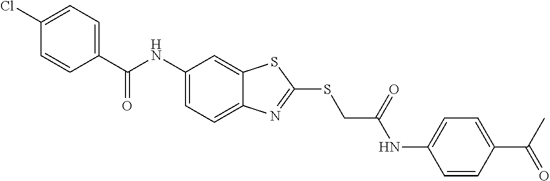 [SMILES: CC(=O)c1ccc(NC(=O)CSc2nc3ccc(NC(=O)c4ccc(Cl)cc4)cc3s2)cc1] C₂₄H₁₈ClN₃O₃S₂ | STK724209 | ++ |
| 115 | 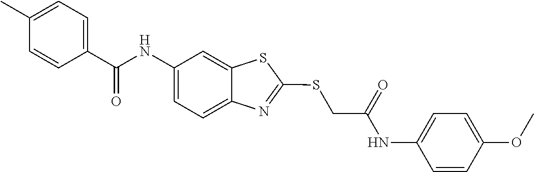 C₂₄H₂₁N₃O₃S₂ | D336-2635 | ++ |
| 116 | 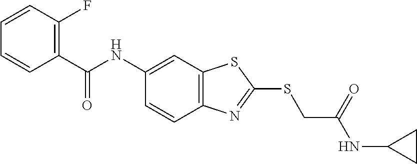 [SMILES: O=C(CSc1nc2ccc(NC(=O)c3ccccc3F)cc2s1)NC1CC1] C₁₉H₁₆FN₃O₂S₂ | BRD- K35346540 | ++ |
| 117 | 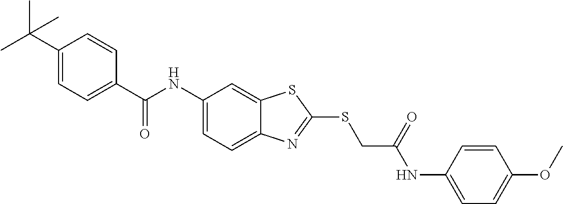 [SMILES: COc1ccc(NC(=O)CSc2nc3ccc(NC(=O)c4ccc(C(C)(C)C)cc4)cc3s2)cc1] C₂₇N₃O₃S₂ | STK724198 | ++ |
| 118 | 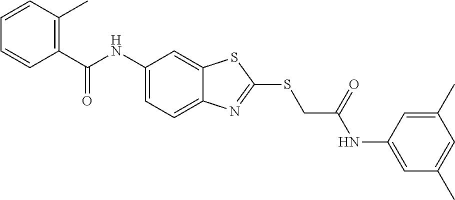 [SMILES: Cc1cc(C)cc(NC(=O)CSc2nc3ccc(NC(=O)c4ccccc4C)cc3s2)c1] C₂₅H₂₃N₃O₂S₂ | D336-2863 | ++ |
| 119 | 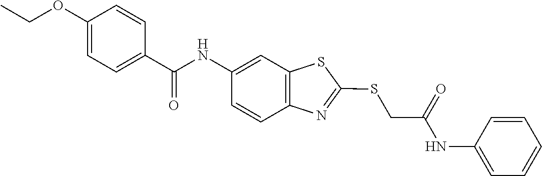 [SMILES: CCOc1ccc(C(=O)Nc2ccc3nc(SCC(=O)Nc4ccccc4)sc3c2)cc1] C₂₄H₂₁N₃O₃S₂ | D336-1903 | ++ |
| 120 | 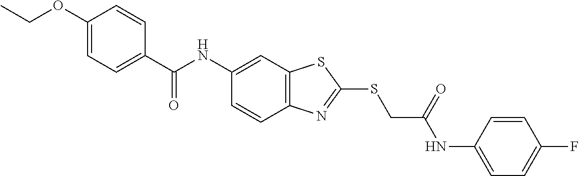 [SMILES: CCOc1ccc(C(=O)Nc2ccc3nc(SCC(=O)Nc4ccc(F)cc4)sc3c2)cc1] C₂₄H₂₀FN₃O₃S₂ | D336-2258 | ++ |
| 121 | 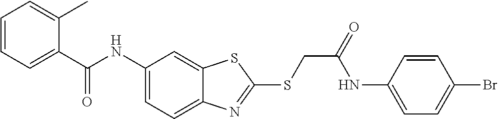 [SMILES: Cc1ccccc1C(=O)Nc1ccc2nc(SCC(=O)Nc3ccc(Br)cc3)sc2c1] C₂₃BrN₃O₂S₂ | STK724174 | ++ |
| 122 | 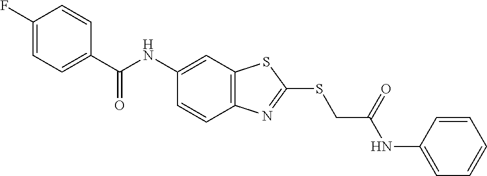 [SMILES: O=C(CSc1nc2ccc(NC(=O)c3ccc(F)cc3)cc2s1)Nc1ccccc1] C₂₂H₁₆FN₃O₂S₂ | AQ- 088/42181560 | ++ |
| 123 | 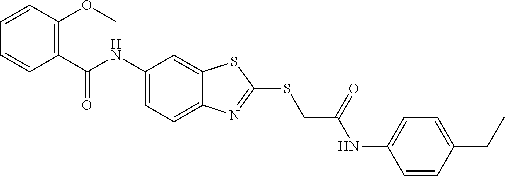 [SMILES: CCc1ccc(NC(=O)CSc2nc3ccc(NC(=O)c4ccccc4OC)cc3s2)cc1] C₂₅H₂₃N₃O₃S₂ | STK757640 | ++ |
| 124 | 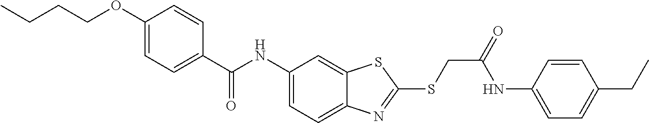 [SMILES: CCCCOc1ccc(C(=O)Nc2ccc3nc(SCC(=O)Nc4ccc(CC)cc4)sc3c2)cc1] C₂₈N₃O₃S₂ | STK755856 | ++ |
| 125 | 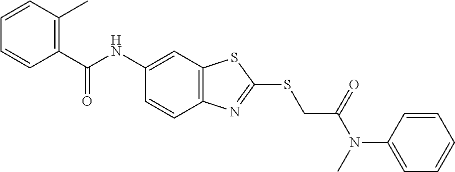 [SMILES: Cc1ccccc1C(=O)Nc1ccc2nc(SCC(=O)N(C)c3ccccc3)sc2c1] C₂₄H₂₁N₃O₂S₂ | D336-1159 | ++ |
| 126 | 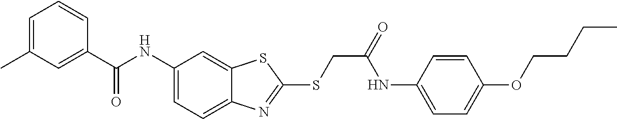 [SMILES: CCCCOc1ccc(NC(=O)CSc2nc3ccc(NC(=O)c4cccc(C)c4)cc3s2)cc1] C₂₇N₃O₃S₂ | STK757974 | ++ |
| 127 | 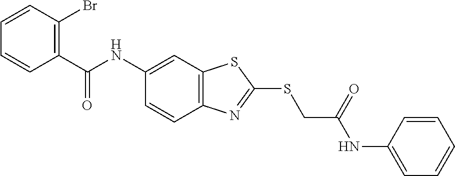 [SMILES: O=C(CSc1nc2ccc(NC(=O)c3ccccc3Br)cc2s1)Nc1ccccc1] C₂₂H₁₆BrN₃O₂S₂ | BRD- K12837429 | ++ |
| 128 | 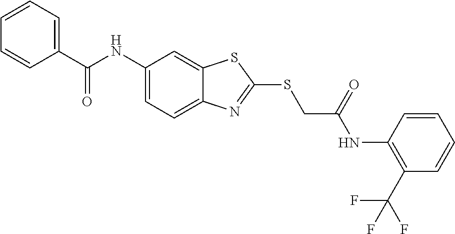 [SMILES: O=C(CSc1nc2ccc(NC(=O)c3ccccc3)cc2s1)Nc1ccccc1C(F)(F)F] C₂₃H₁₆F₃N₃O₂S₂ | BRD- K41731087 | ++ |
| 129 | 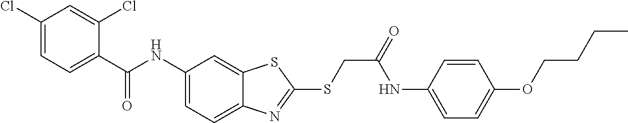 [SMILES: CCCCOc1ccc(NC(=O)CSc2nc3ccc(NC(=O)c4ccc(Cl)cc4Cl)cc3s2)cc1] C₂₆C₁₂N₃O₃S₂ | STK674756 | ++ |
| 130 | 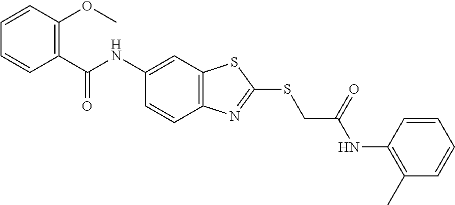 [SMILES: COc1ccccc1C(=O)Nc1ccc2nc(SCC(=O)Nc3ccccc3C)sc2c1] C₂₄H₂₁N₃O₃S₂ | D336-1958 | ++ |
| 131 | 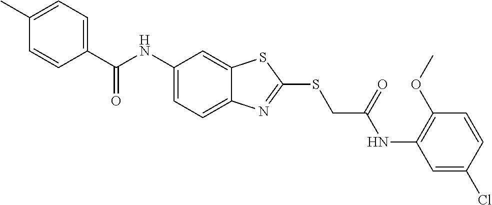 [SMILES: COc1ccc(Cl)cc1NC(=O)CSc1nc2ccc(NC(=O)c3ccc(C)cc3)cc2s1] C₂₄H₂₀ClN₃O₃S₂ | BRD- K21034661 | ++ |
| 132 | 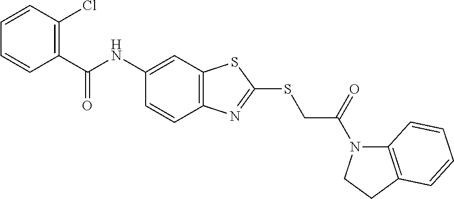 [SMILES: O=C(Nc1ccc2nc(SCC(=O)N3CCc4ccccc43)sc2c1)c1ccccc1Cl] C₂₄H₁₈ClN₃O₂S₂ | BRD- K00225507 | ++ |
| 133 | 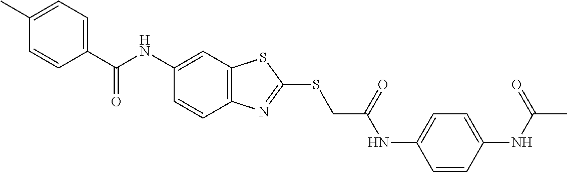 [SMILES: CC(=O)Nc1ccc(NC(=O)CSc2nc3ccc(NC(=O)c4ccc(C)cc4)cc3s2)cc1] | AQ 088/42014309 | ++ |
| 134 | 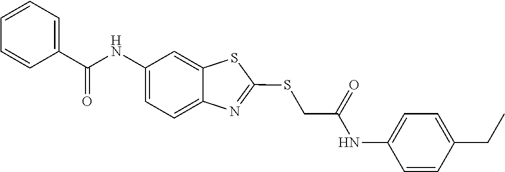 [SMILES: CCc1ccc(NC(=O)CSc2nc3ccc(NC(=O)c4ccccc4)cc3s2)cc1] C₂₄H₂₁N₃O₂S₂ | STK125680 | ++ |
| 135 | 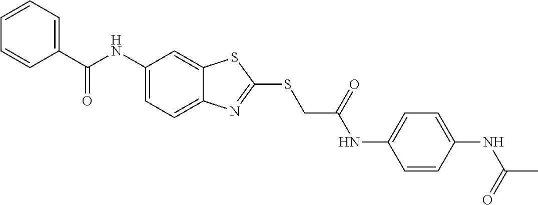 [SMILES: CC(=O)Nc1ccc(NC(=O)CSc2nc3ccc(NC(=O)c4ccccc4)cc3s2)cc1] C₂₄H₂₀N₄O₃S₂ | STK171814 | ++ |
| 136 | 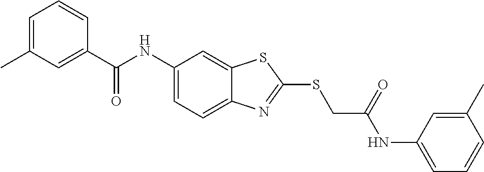 C₂₄H₂₁N₃O₂S₂ | STK232865 | ++ |
| 137 | 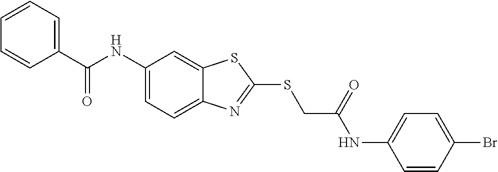 [SMILES: O=C(CSc1nc2ccc(NC(=O)c3ccccc3)cc2s1)Nc1ccc(Br)cc1] C₂₂H₁₆BrN₃O₂S₂ | STK724140 | ++ |
| 138 | 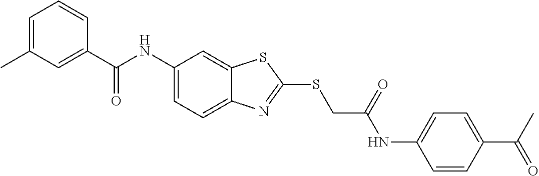 [SMILES: CC(=O)c1ccc(NC(=O)CSc2nc3ccc(NC(=O)c4cccc(C)c4)cc3s2)cc1] C₂₅H₂₁N₃O₃S₂ | STK724284 | ++ |
| 139 | 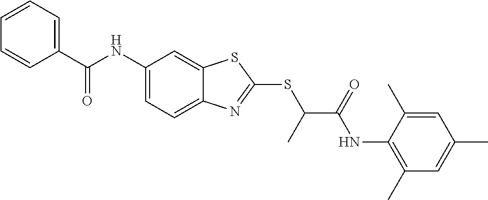 [SMILES: Cc1cc(C)c(NC(=O)C(C)Sc2nc3ccc(NC(=O)c4ccccc4)cc3s2)c(C)c1] C₂₆H₂₅N₃O₂S₂ | STK742529 | ++ |
| 140 | 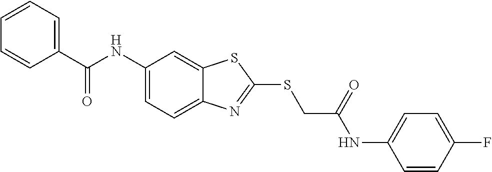 [SMILES: O=C(CSc1nc2ccc(NC(=O)c3ccccc3)cc2s1)Nc1ccc(F)cc1] C₂₂H₁₆FN₃O₂S₂ | STK742619 | ++ |
| 141 | 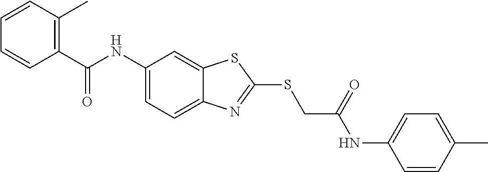 [SMILES: Cc1ccc(NC(=O)CSc2nc3ccc(NC(=O)c4ccccc4C)cc3s2)cc1] C₂₄H₂₁N₃O₂S₂ | D336-2082 | ++ |
| 142 | 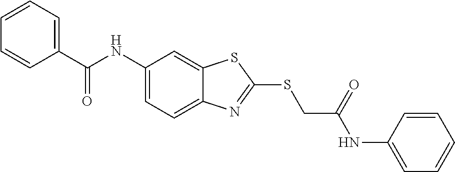 [SMILES: O=C(CSc1nc2ccc(NC(=O)c3ccccc3)cc2s1)Nc1ccccc1] C₂₂H₁₇N₃O₂S₂ | D336-1848 | + |
| 143 | 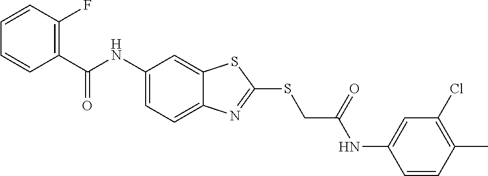 [SMILES: Cc1ccc(NC(=O)CSc2nc3ccc(NC(=O)c4ccccc4F)cc3s2)cc1Cl] C₂₃H₁₇ClFN₃O₂S₂ | STK724163 | + |
| 144 | 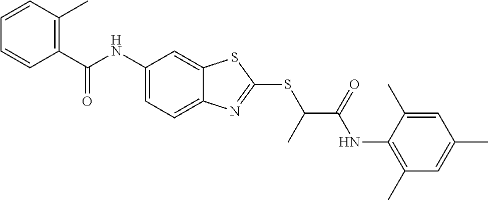 [SMILES: Cc1cc(C)c(NC(=O)C(C)Sc2nc3ccc(NC(=O)c4ccccc4C)cc3s2)c(C)c1] C₂₇H₂₇N₃O₂S₂ | STK724176 | + |
| 145 | 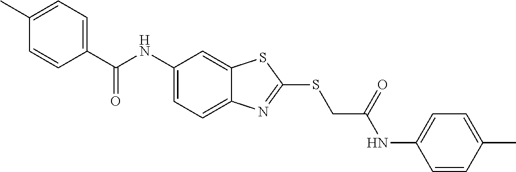 C₂₄H₂₁N₃O₂S₂ | D336-2067 | + |
| 146 | 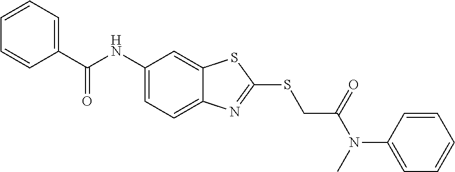 [SMILES: CN(C(=O)CSc1nc2ccc(NC(=O)c3ccccc3)cc2s1)c1ccccc1] C₂₃H₁₉N₃O₂S₂ | D336-1138 | + |
| 147 | 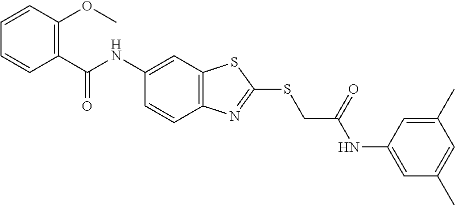 [SMILES: COc1ccccc1C(=O)Nc1ccc2nc(SCC(=O)Nc3cc(C)cc(C)c3)sc2c1] C₂₅H₂₃N₃O₃S₂ | D336-2881 | + |
| 148 | 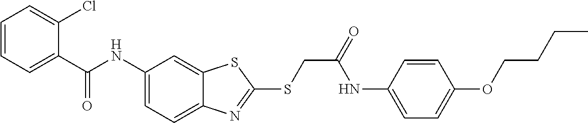 [SMILES: CCCCOc1ccc(NC(=O)CSc2nc3ccc(NC(=O)c4ccccc4Cl)cc3s2)cc1] C₂₆ClN₃O₃S₂ | STK674367 | + |
| 149 | 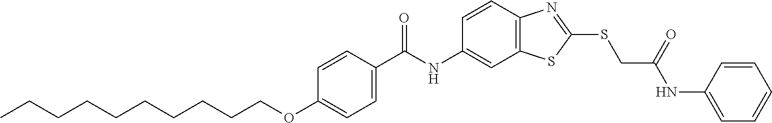 [SMILES: CCCCCCCCCCOc1ccc(C(=O)Nc2ccc3nc(SCC(=O)Nc4ccccc4)sc3c2)cc1] C₃₂H₃₇N₃O₃S₂ | 8007-7681 | + |
| 150 | 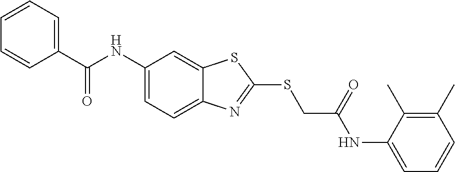 [SMILES: Cc1cccc(NC(=O)CSc2nc3ccc(NC(=O)c4ccccc4)cc3s2)c1C] C₂₄H₂₁N₃O₂S₂ | 6352863 | + |
| 151 | 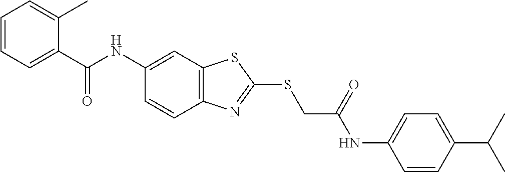 [SMILES: Cc1ccccc1C(=O)Nc1ccc2nc(SCC(=O)Nc3ccc(C(C)C)cc3)sc2c1] C₂₆H₂₅N₃O₂S₂ | AG- 205/12084125 | + |
| 152 | 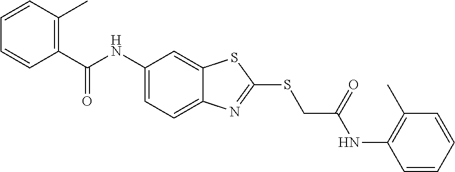 [SMILES: Cc1ccccc1NC(=O)CSc1nc2ccc(NC(=O)c3ccccc3C)cc2s1] C₂₄H₂₁N₃O₂S₂ | D336-1940 | + |
| 153 | 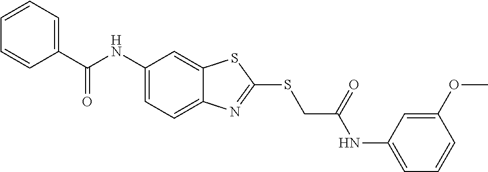 [SMILES: COc1cccc(NC(=O)CSc2nc3ccc(NC(=O)c4ccccc4)cc3s2)c1] C₂₃H₁₉N₃O₃S₂ | D336-2558 | + |
| 154 | 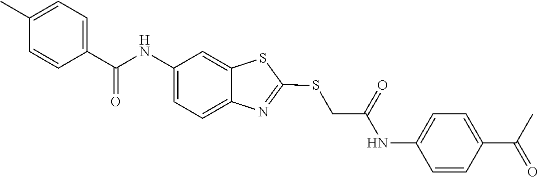 [SMILES: CC(=O)c1ccc(NC(=O)CSc2nc3ccc(NC(=O)c4ccc(C)cc4)cc3s2)cc1] C₂₅H₂₁N₃O₃S₂ | STK032422 | + |
| 155 | 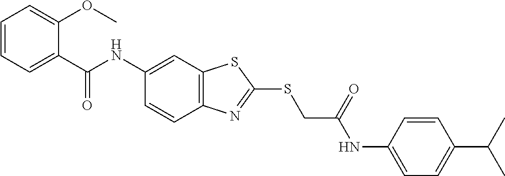 [SMILES: COc1ccccc1C(=O)Nc1ccc2nc(SCC(=O)Nc3ccc(C(C)C)cc3)sc2c1] C₂₆H₂₅N₃O₃S₂ | AG- 205/12084061 | + |
| 156 | 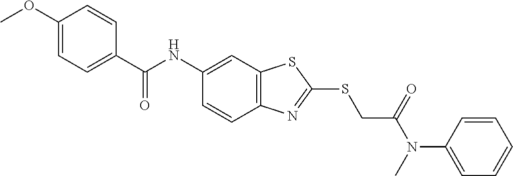 [SMILES: COc1ccc(C(=O)Nc2ccc3nc(SCC(=O)N(C)c4ccccc4)sc3c2)cc1] C₂₄H₂₁N₃O₃S₂ | D336-1178 | + |
| 157 | 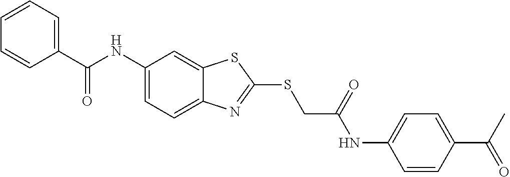 [SMILES: CC(=O)c1ccc(NC(=O)CSc2nc3ccc(NC(=O)c4ccccc4)cc3s2)cc1] C₂₄H₁₉N₃O₃S₂ | STK724141 | + |
| 158 | 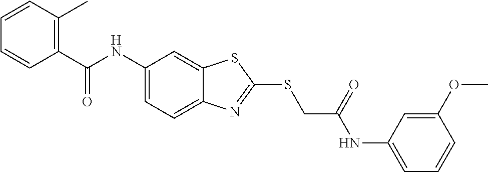 [SMILES: COc1cccc(NC(=O)CSc2nc3ccc(NC(=O)c4ccccc4C)cc3s2)c1] C₂₄H₂₁N₃O₃S₂ | D336-2579 | + |
| 159 | 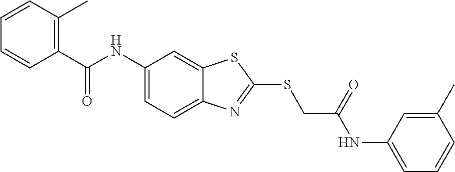 [SMILES: Cc1cccc(NC(=O)CSc2nc3ccc(NC(=O)c4ccccc4C)cc3s2)c1] C₂₄H₂₁N₃O₂S₂ | D336-2011 | + |
| 160 | 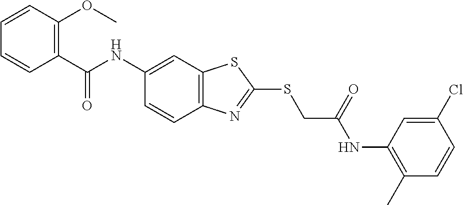 [SMILES: COc1ccccc1C(=O)Nc1ccc2nc(SCC(=O)Nc3cc(Cl)ccc3C)sc2c1] C₂₄H₂₀ClN₃O₃S₂ | STK759010 | + |
| 161 | 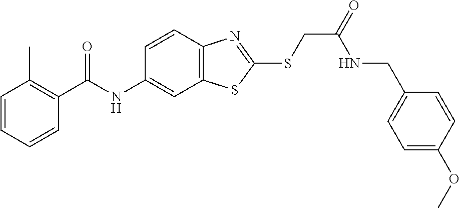 [SMILES: COc1ccc(CNC(=O)CSc2nc3ccc(NC(=O)c4ccccc4C)cc3s2)cc1] C₂₅N₃O₃S₂ | STK054868 | + |
| 162 | 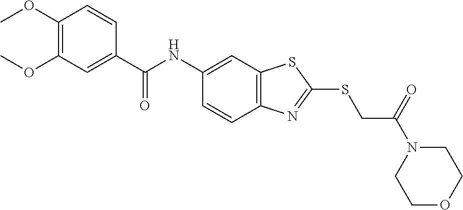 [SMILES: COc1ccc(C(=O)Nc2ccc3nc(SCC(=O)N4CCOCC4)sc3c2)cc1OC] C₂₂H₂₃N₃O₅S₂ | BRD- K59481447 | + |
| 163 | 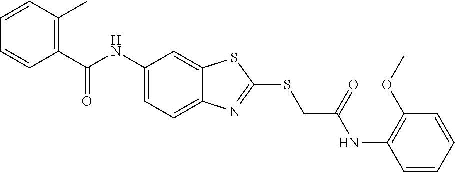 [SMILES: COc1ccccc1NC(=O)CSc1nc2ccc(NC(=O)c3ccccc3C)cc2s1] C₂₄H₂₁N₃O₃S₂ | D336-2508 | + |
| 164 | 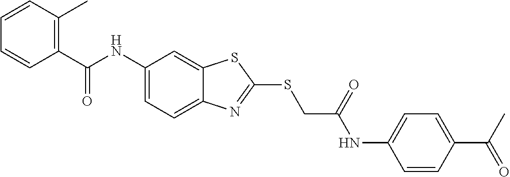 [SMILES: CC(=O)c1ccc(NC(=O)CSc2nc3ccc(NC(=O)c4ccccc4C)cc3s2)cc1] C₂₅H₂₁N₃O₃S₂ | STK724175 | + |
| 165 | 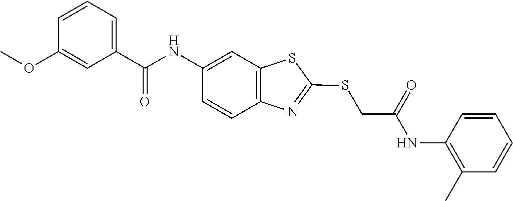 [SMILES: COc1cccc(C(=O)Nc2ccc3nc(SCC(=O)Nc4ccccc4C)sc3c2)c1] C₂₄H₂₁N₃O₃S₂ | D336-1961 | + |
| 166 | 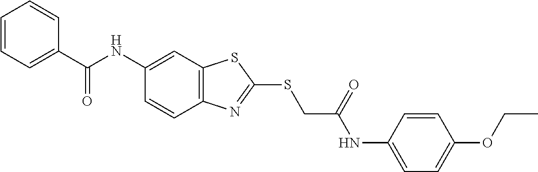 [SMILES: CCOc1ccc(NC(=O)CSc2nc3ccc(NC(=O)c4ccccc4)cc3s2)cc1] C₂₄H₂₁N₃O₃S₂ | AN- 648/15102292 | + |
| 167 | 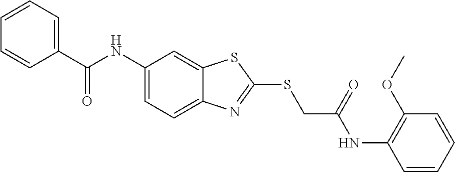 [SMILES: COc1ccccc1NC(=O)CSc1nc2ccc(NC(=O)c3ccccc3)cc2s1] C₂₃H₁₉N₃O₃S₂ | STK724137 | + |
| 168 | 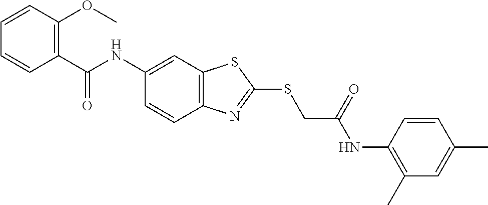 [SMILES: COc1ccccc1C(=O)Nc1ccc2nc(SCC(=O)Nc3ccc(C)cc3C)sc2c1] C₂₅H₂₃N₃O₃S₂ | BRD- K27135406 | + |
| 169 | 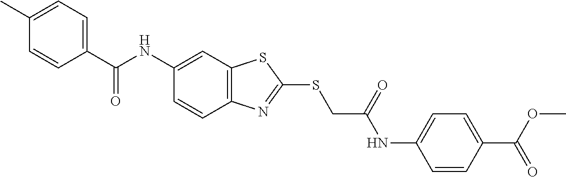 C₂₅H₂₁N₃O₄S₂ | AM- 879/14775325 | + |
| 170 | 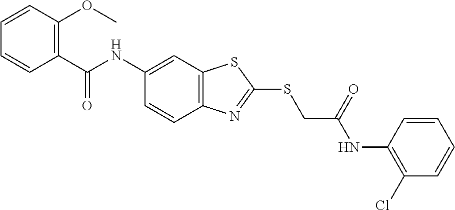 [SMILES: COc1ccccc1C(=O)Nc1ccc2nc(SCC(=O)Nc3ccccc3Cl)sc2c1] C₂₃H₁₈ClN₃O₃S₂ | D336-2313 | + |
| 171 | 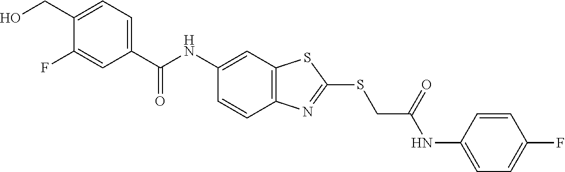 [SMILES: O=C(CSc1nc2ccc(NC(=O)c3ccc(CO)c(F)c3)cc2s1)Nc1ccc(F)cc1] | IBX-101 | +++ |
| 172 | 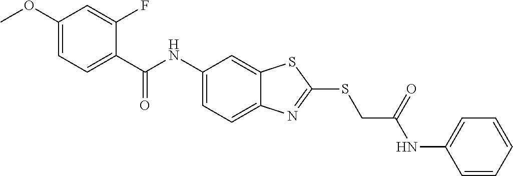 [SMILES: COc1ccc(C(=O)Nc2ccc3nc(SCC(=O)Nc4ccccc4)sc3c2)c(F)c1] | IBX-102 | +++ |
| 173 | 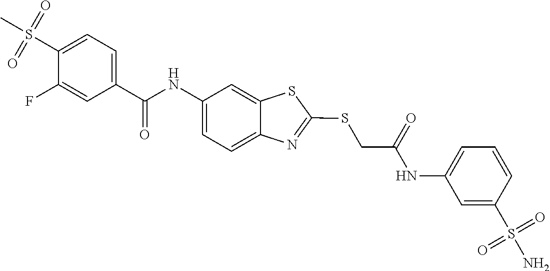 [SMILES: CS(=O)(=O)c1ccc(C(=O)Nc2ccc3nc(SCC(=O)Nc4cccc(S(N)(=O)=O)c4)sc3c2)cc1F] | IBX-103 | ++ |
| 174 | 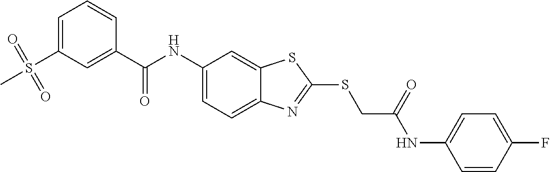 [SMILES: CS(=O)(=O)c1cccc(C(=O)Nc2ccc3nc(SCC(=O)Nc4ccc(F)cc4)sc3c2)c1] | IBX-104 | +++ |

1-25. (canceled)

26. A method of treating a senescence-associated disease or disorder or an age-related disease or disorder in a subject comprising, administering to the subject a compound of Formula (I): 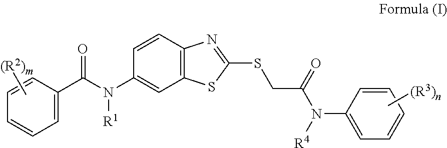
or a pharmaceutically acceptable salt thereof, wherein;
R¹ and R⁴ are each independently selected from hydrogen and C₁₋₆ alkyl;
each R² and R³ is independently selected from halogen, —OR⁵, SR⁵, —N(R⁵)₂, —C(O)R⁶, —C(O)N(R⁵)₂, —N(R⁵)C(O)R⁶, —C(O)OR⁵, —OC(O)R⁶, —S(O)₂R⁶, —S(O)₂N(R⁵)₂, —NO₂, —CN, C₁₋₆ alkyl, C₂₋₆ alkenyl, and C₂₋₆ alkynyl; wherein each C₁₋₆ alkyl, C₂₋₆ alkenyl, and C₂₋₆ alkynyl is optionally substituted with one or more substituents independently selected from halogen, —OR⁵, —SR⁵, —N(R⁵)₂, —C(O)R⁶, —C(O)N(R⁵)₂, —N(R⁵)C(O)R⁶, —C(O)OR⁵, —OC(O)R⁶, —NO₂, ═O, and —CN;
each R⁵ is independently selected from: hydrogen; and C₁₋₆ alkyl optionally substituted with one more substituents independently selected from halogen, —O—C₁₋₆ alkyl, —O—C₁₋₆ haloalkyl, —NH₂—NO₂, —O, and —CN;
each R⁶ is independently selected from: C₁₋₆ alkyl optionally substituted with one more substituents independently selected from halogen, —O—C₁₋₆ alkyl, —O—C₁₋₆ haloalkyl, —NH₂—NO₂, ═O, and —CN;
m is 0, 1, 2, 3, 4, or 5; and
n is 0, 1, 2, 3, 4, or 5.

27. The method of claim 26, comprising decreasing senescent cell burden and/or mRNA expression of senescence-associated genes in the subject.

28. The method of claim 27, wherein:
each R² and R³ is independently selected from the group consisting of halogen, —OR⁵, —N(R⁵)₂, and C₁₋₆ alkyl; wherein each C₁₋₆ alkyl, is optionally substituted with one or more substituents independently selected from halogen, —OR⁵, —N(R⁵)₂, —C(O)R⁶, —C(O)N(R⁵)₂, —N(R⁵)C(O)R⁶, —C(O)OR⁵, —OC(O)R⁶, ═O, and —CN;
each R⁵ is independently selected from: hydrogen; and C₁₋₆ alkyl optionally substituted with one more substituents independently selected from halogen, —O—C₁₋₆ alkyl, —O—C₁₋₆ haloalkyl, —NH₂—O, and —CN; and
each R⁶ is independently selected from: C₁₋₆ alkyl optionally substituted with one more substituents independently selected from halogen, —O—C₁₋₆ alkyl, —O—C₁₋₆ haloalkyl, —NH₂—O, and —CN.

29. The method of claim 28, wherein:
R⁵ is hydrogen or C₁₋₆ alkyl; and R⁶ is C₁₋₆ alkyl.

30. The method of claim 27, wherein:
R¹ and R⁴ are each hydrogen; and
each R² and R³ is independently halogen, —O—C₁₋₆ alkyl, C₁₋₆ alkyl, —O—C₁₋₆ haloalkyl, or C₁₋₆ haloalkyl;
m is selected from 1 or 2; and
n is 0, 1, 2, or 3.

31. The method of claim 30, wherein:
R¹ and R⁴ are each hydrogen; and
each R² is independently halogen, —O—C₁₋₃ alkyl, C₁₋₃ alkyl, —O—C₁₋₃ haloalkyl, or C₁₋₃ haloalkyl;
each R³ is independently halogen, C₁₋₃ alkyl, and C₁₋₃ haloalkyl;
m is selected from 1 or 2; and
n is 0, 1, or 3.

32. The method of claim 30, wherein each R² and R³ is independently selected from —F, —Cl, —OCH₃, —OCH₂CH₃, —CH₃, and —CH₂CH₃.

33. The method of claim 27, wherein the compound or the pharmaceutically acceptable salt therefor further comprises a pharmaceutically acceptable excipient.

34. The method of claim 27, where the compound of Formula (I) is represented by one of the following structures: 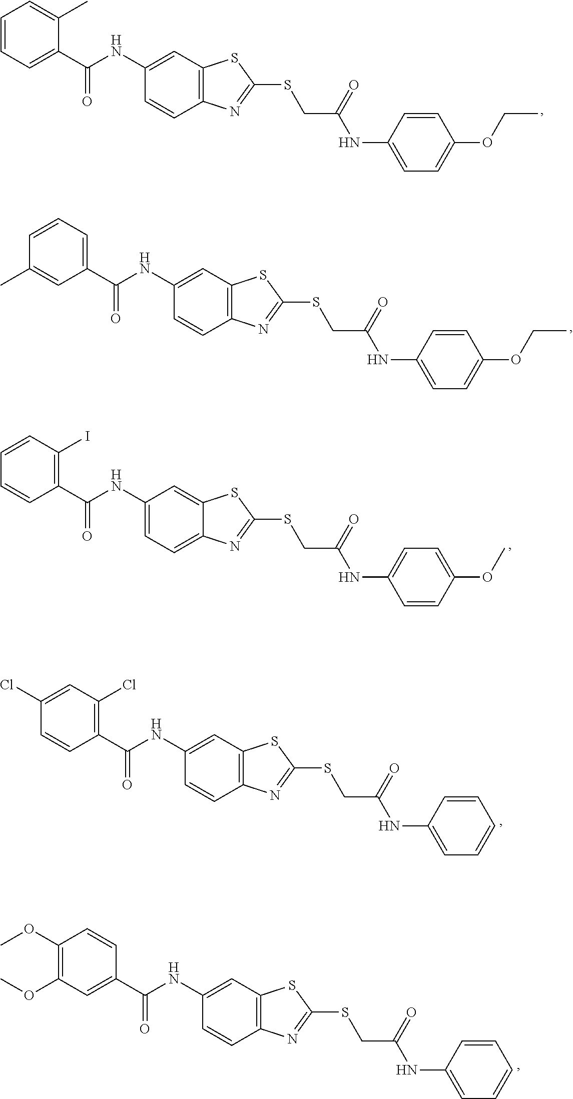 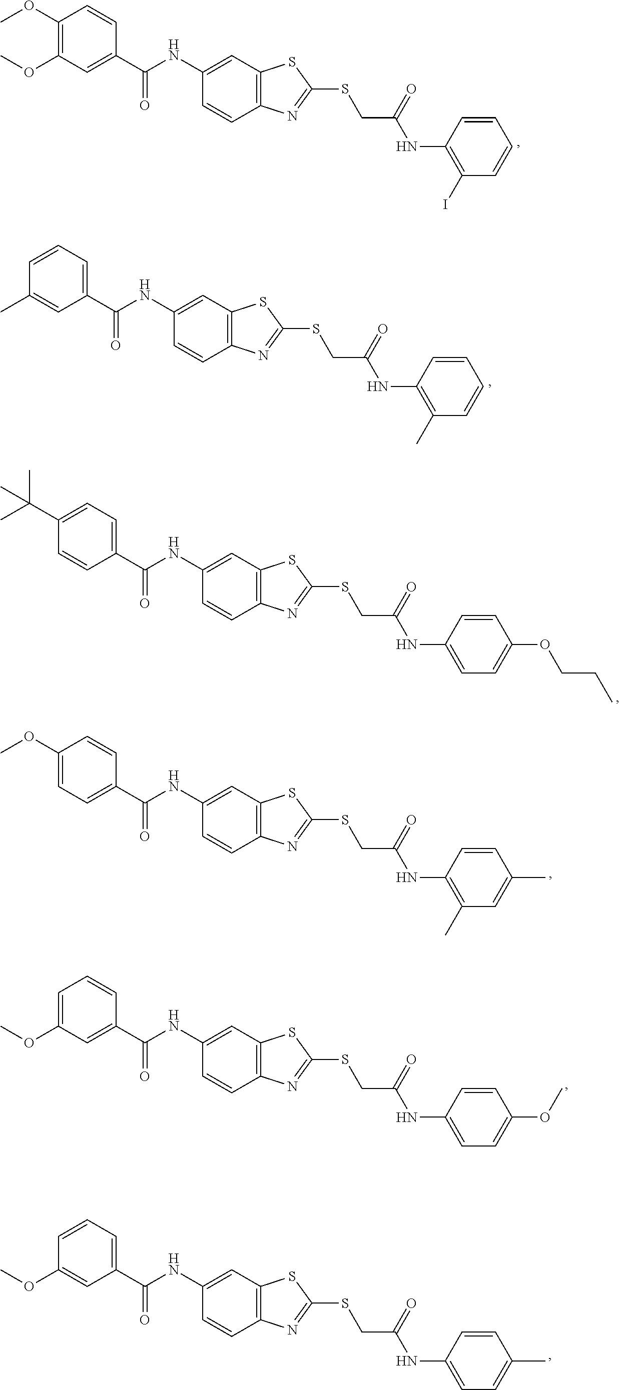 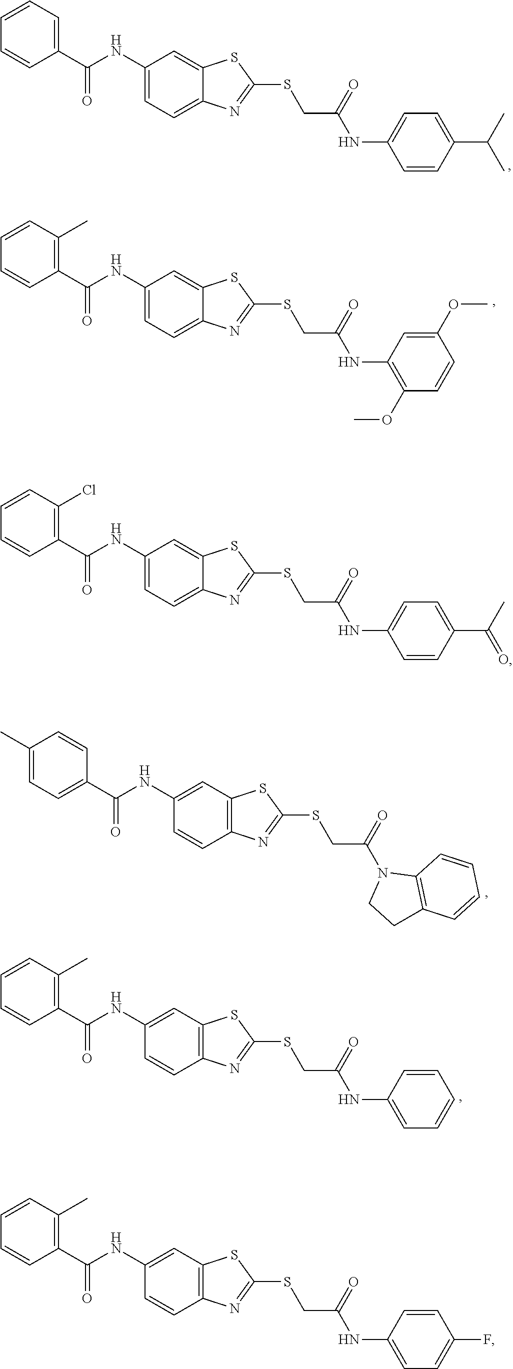 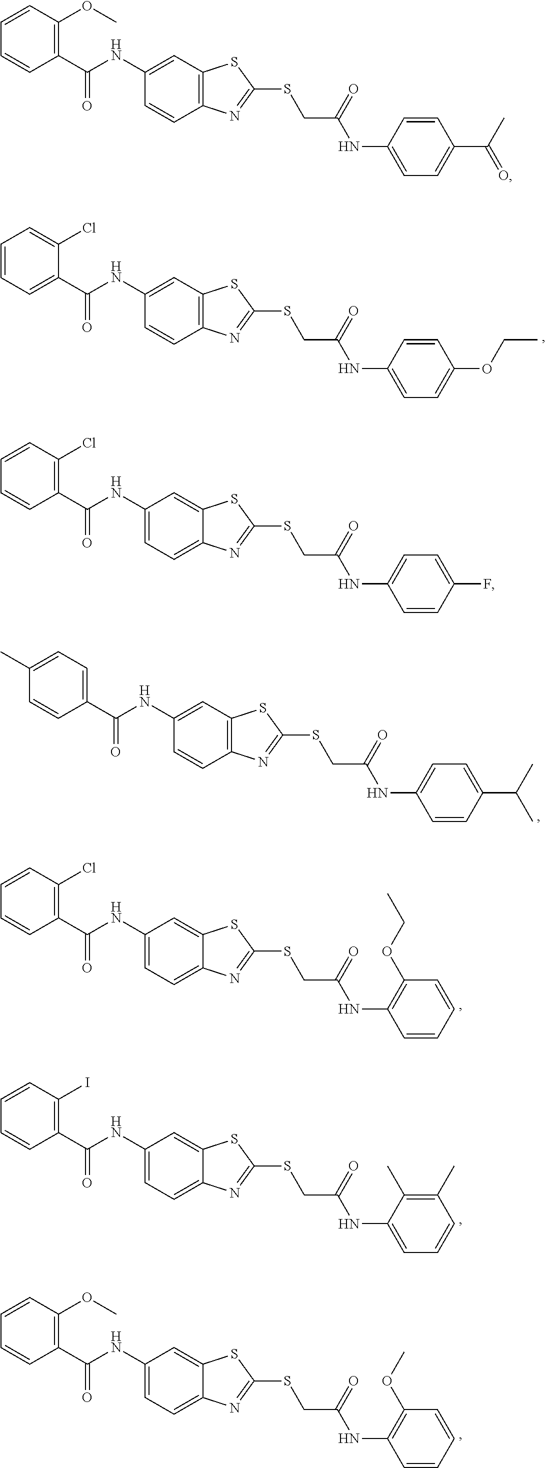 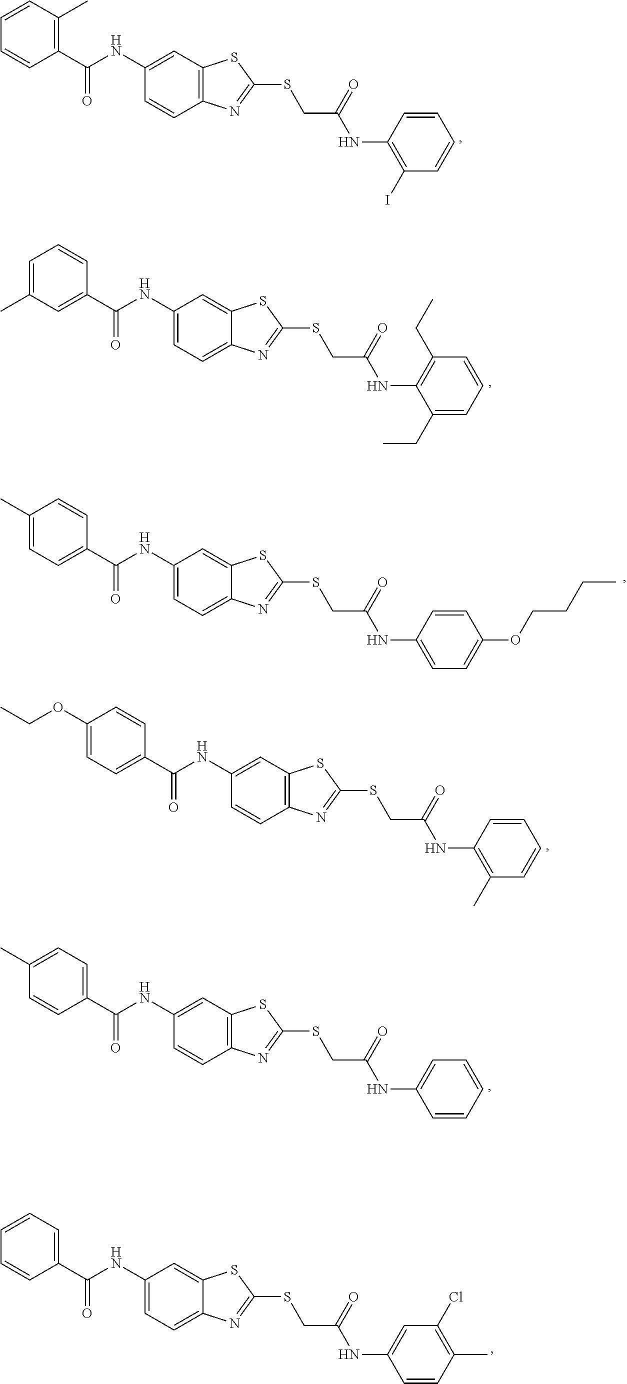 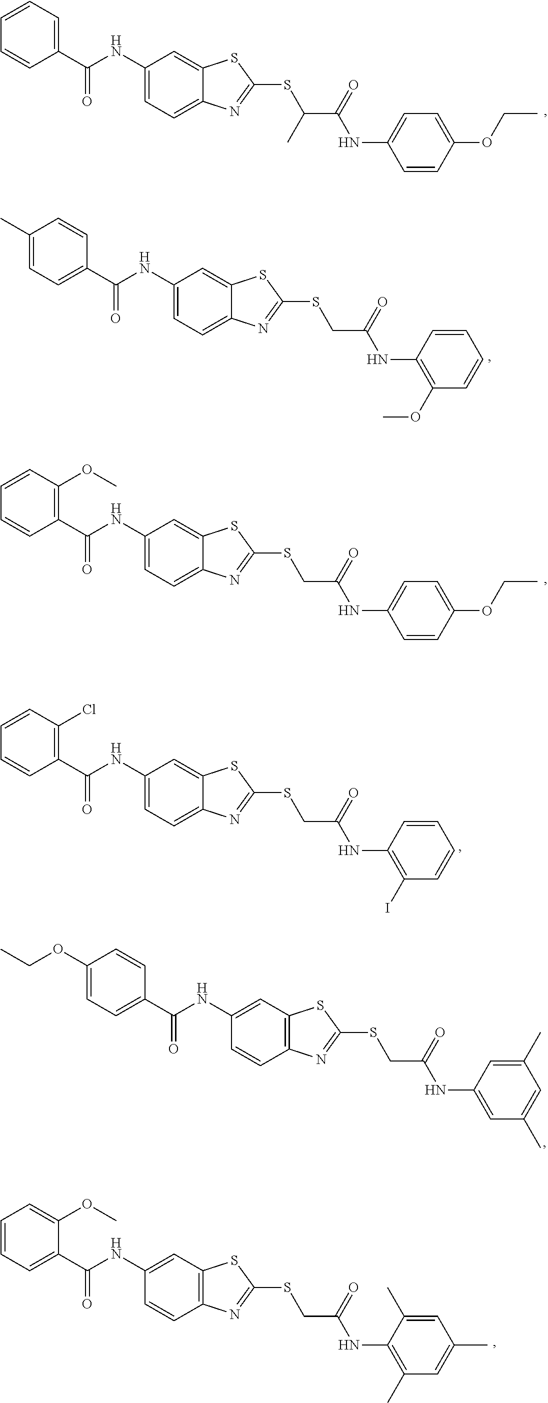 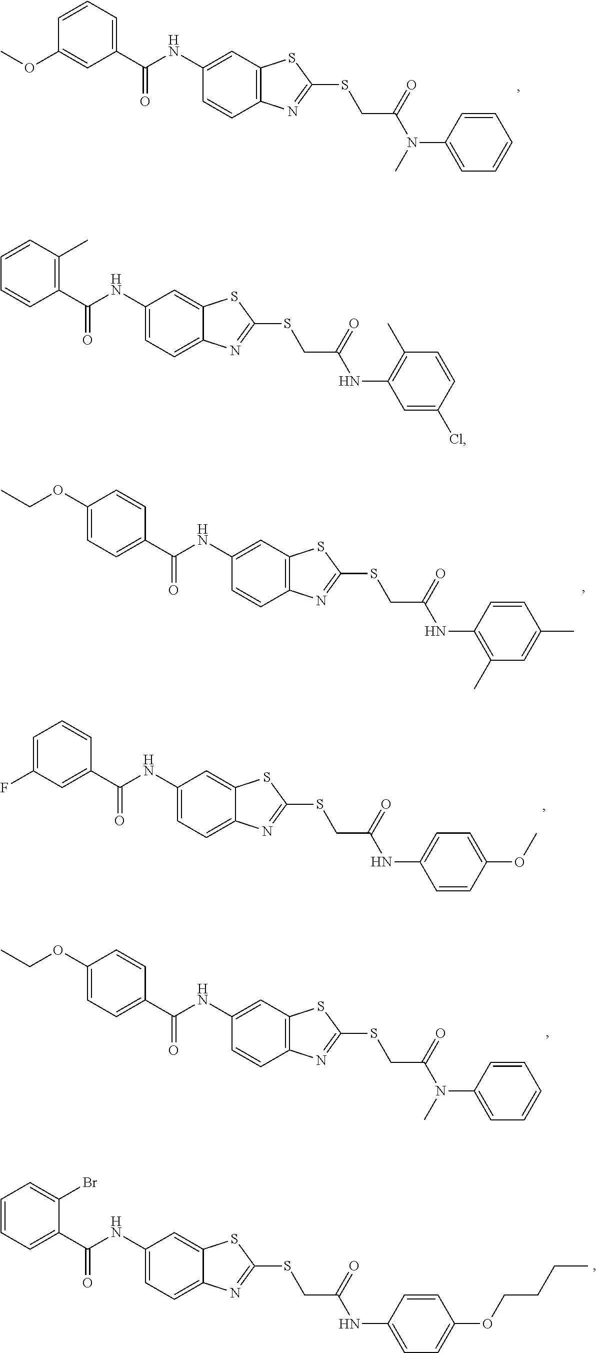 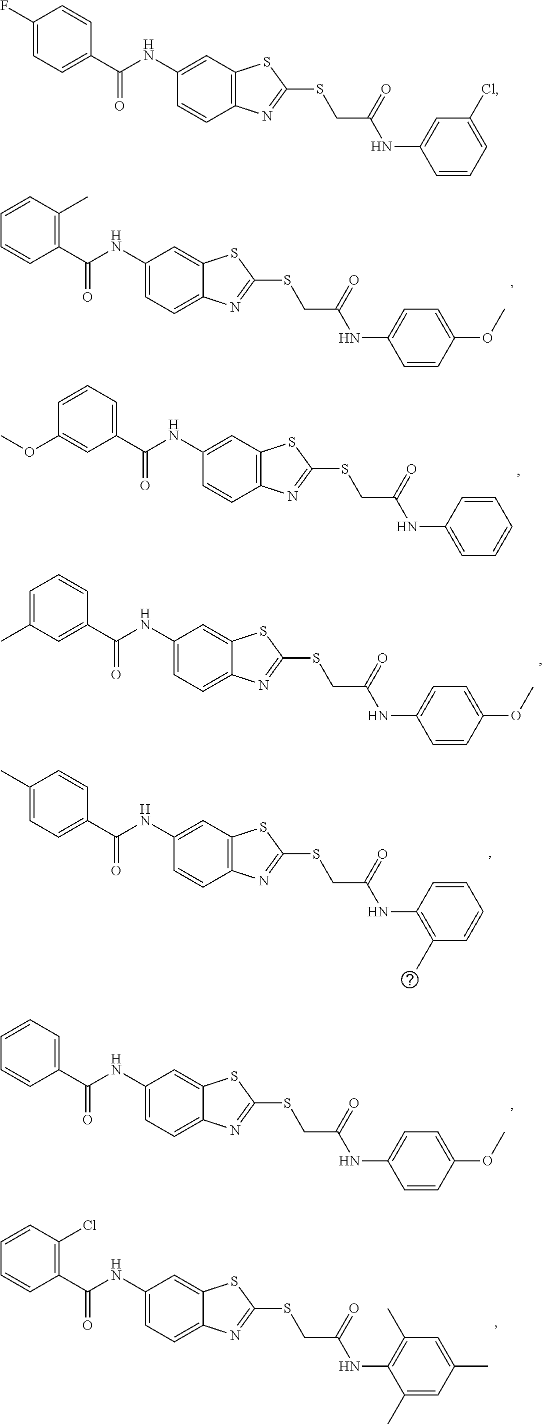 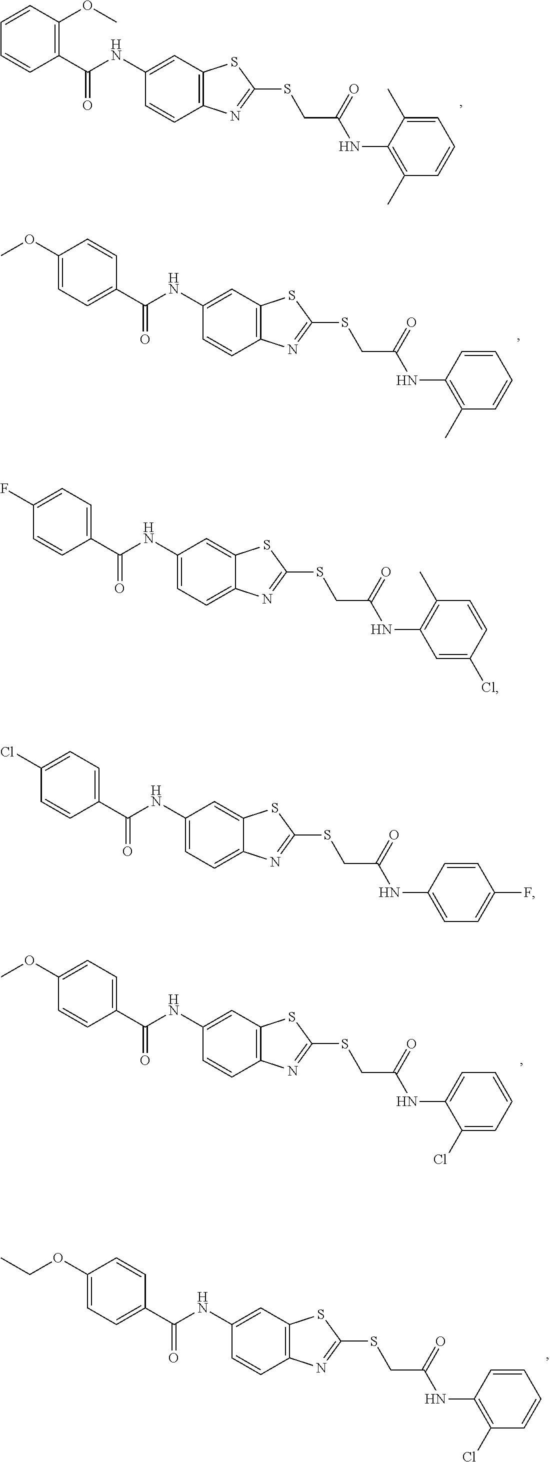 [SMILES: COc1ccccc1C(=O)Nc1ccc2nc(SCC(=O)Nc3c(C)cccc3C)sc2c1 ; COc1ccc(C(=O)Nc2ccc3nc(SCC(=O)Nc4ccccc4C)sc3c2)cc1 ; Cc1ccc(Cl)cc1NC(=O)CSc1nc2ccc(NC(=O)c3ccc(F)cc3)cc2s1 ; O=C(CSc1nc2ccc(NC(=O)c3ccc(Cl)cc3)cc2s1)Nc1ccc(F)cc1 ; COc1ccc(C(=O)Nc2ccc3nc(SCC(=O)Nc4ccccc4Cl)sc3c2)cc1 ; CCOc1ccc(C(=O)Nc2ccc3nc(SCC(=O)Nc4ccccc4Cl)sc3c2)cc1] 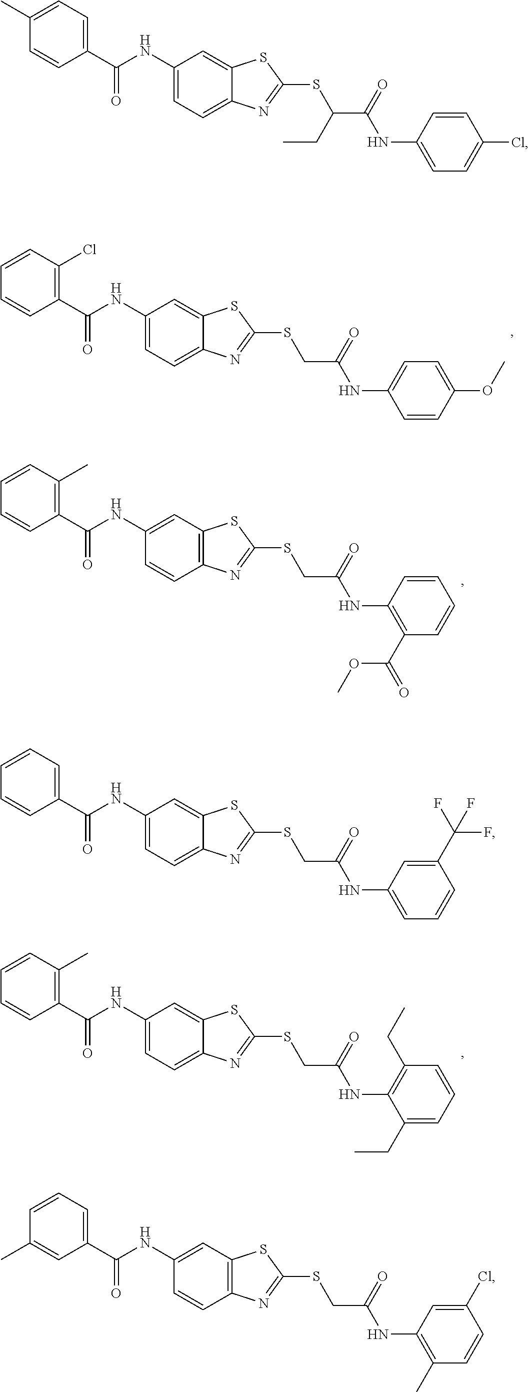 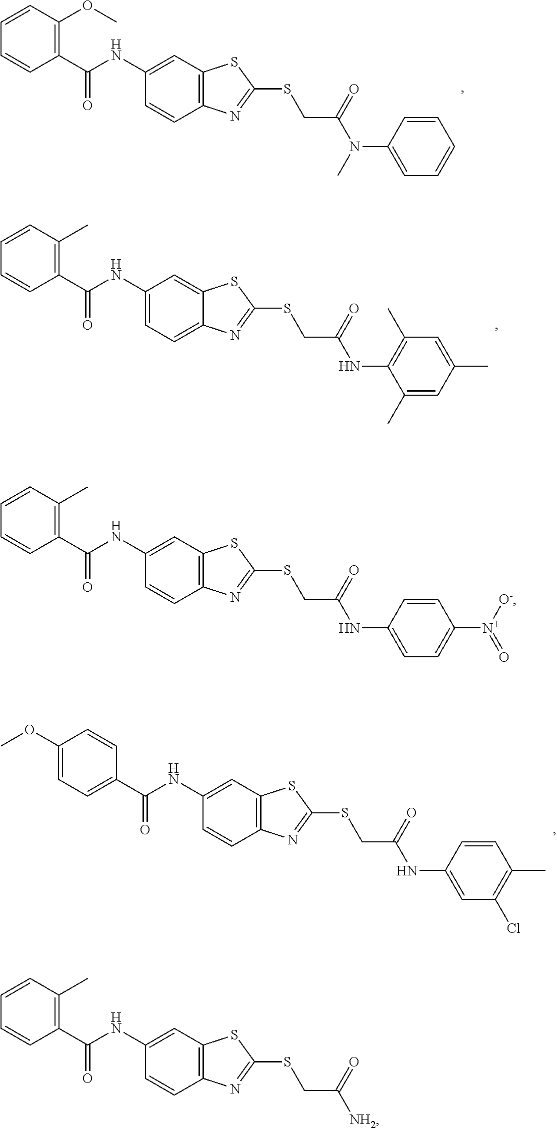 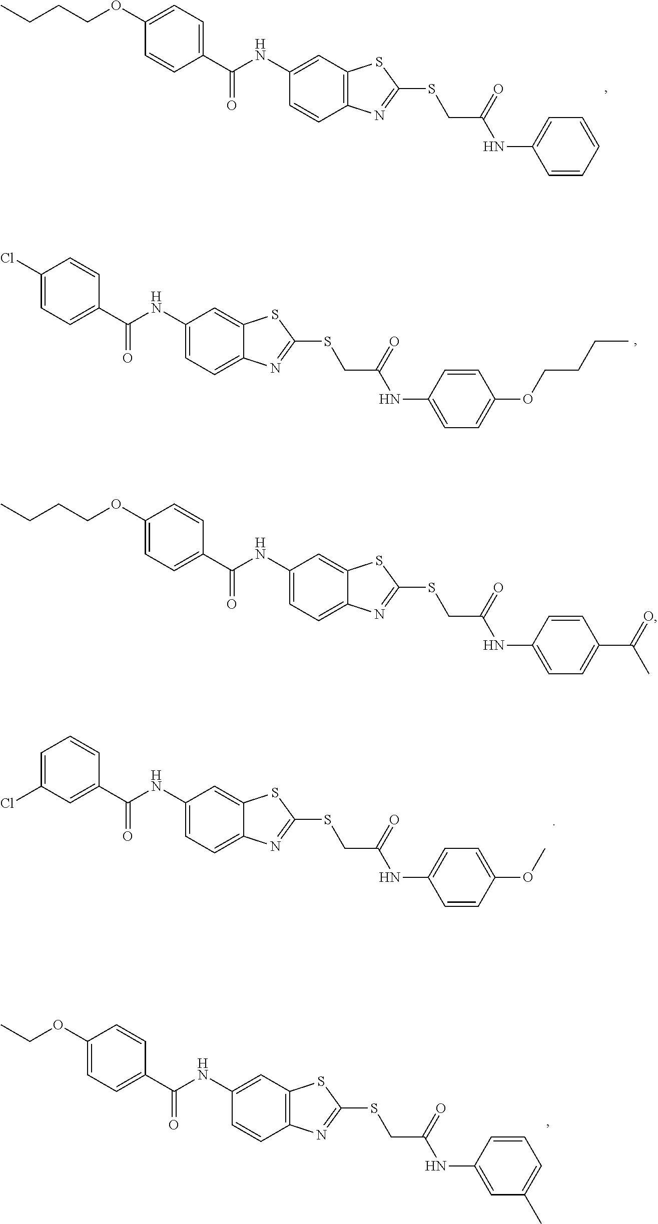 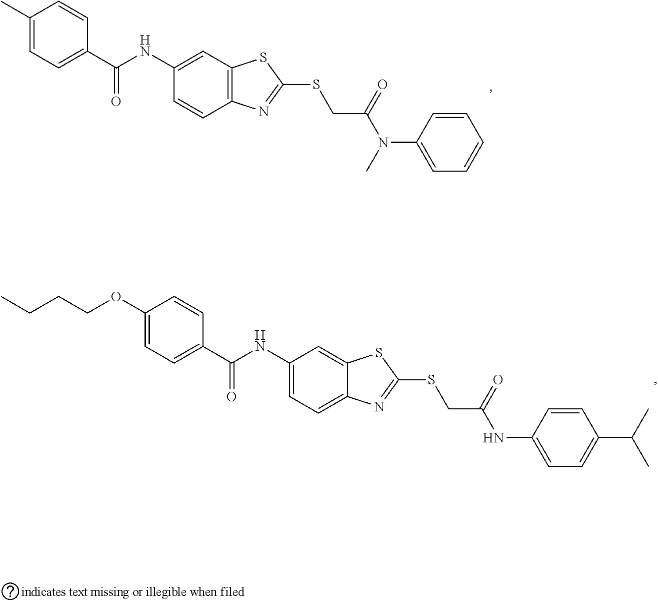 [SMILES: Cc1ccc(C(=O)Nc2ccc3nc(SCC(=O)N(C)c4ccccc4)sc3c2)cc1 ; CCCCOc1ccc(C(=O)Nc2ccc3nc(SCC(=O)Nc4ccc(C(C)C)cc4)sc3c2)cc1] 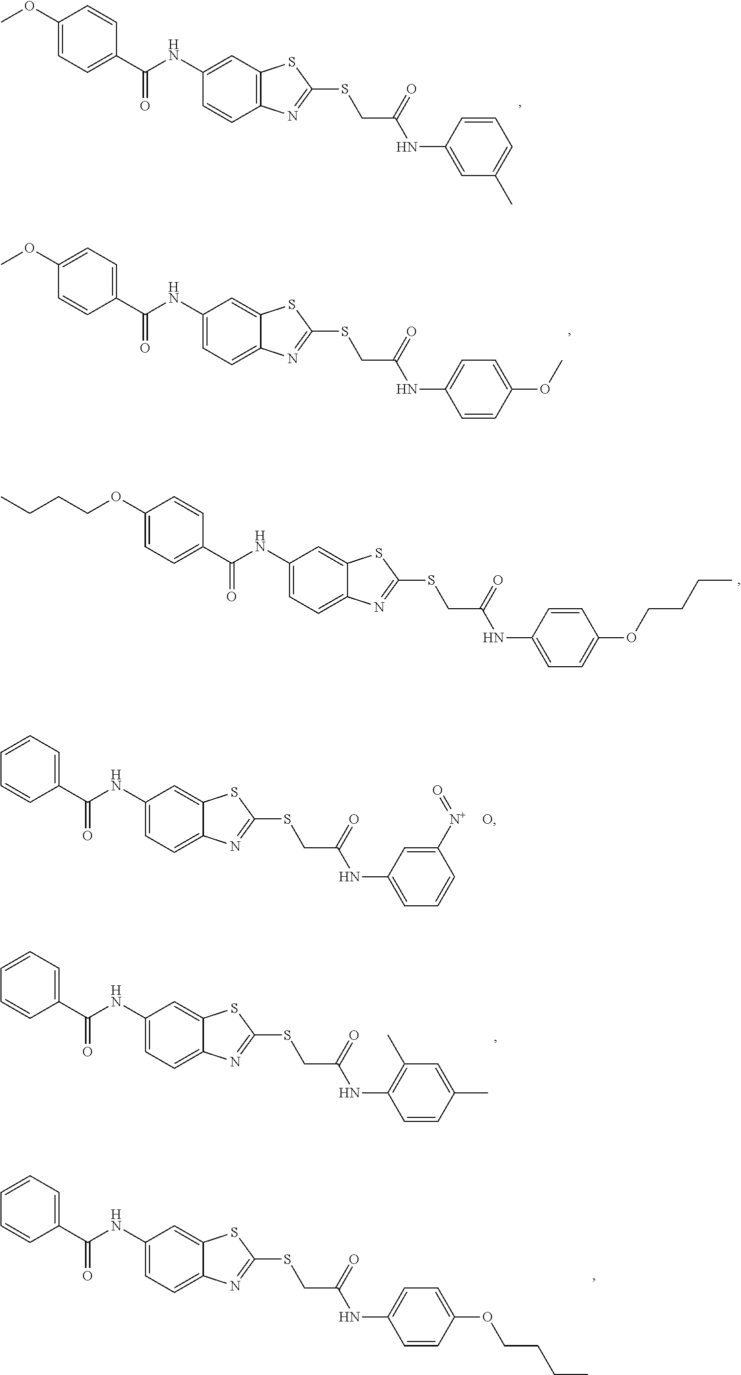 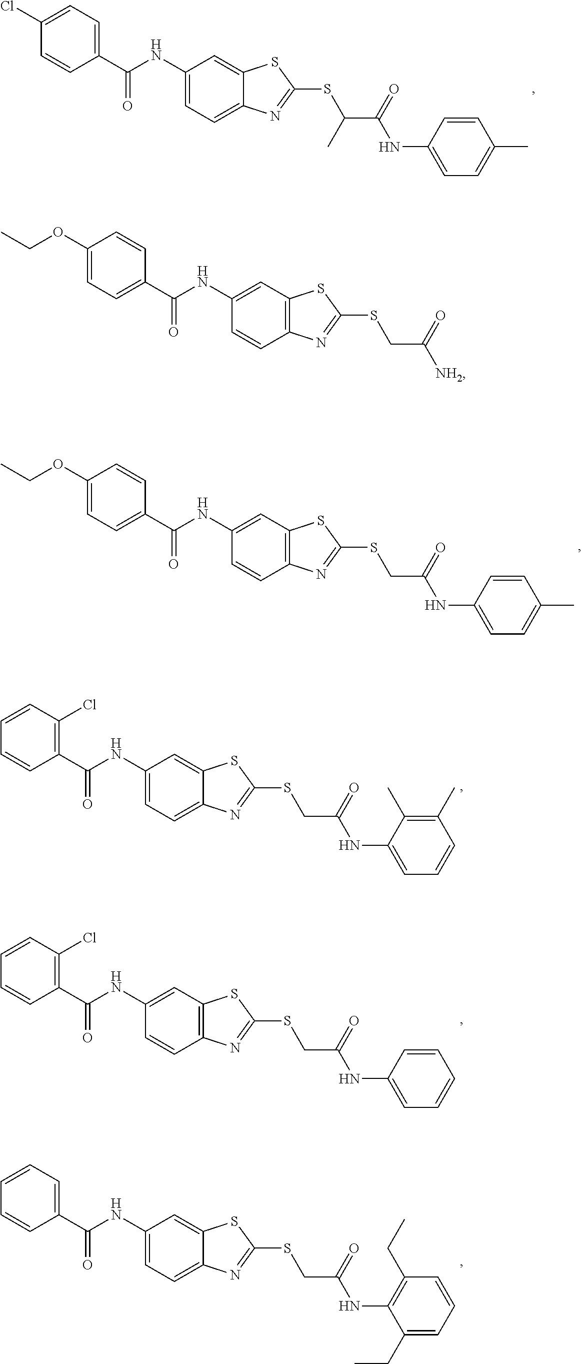 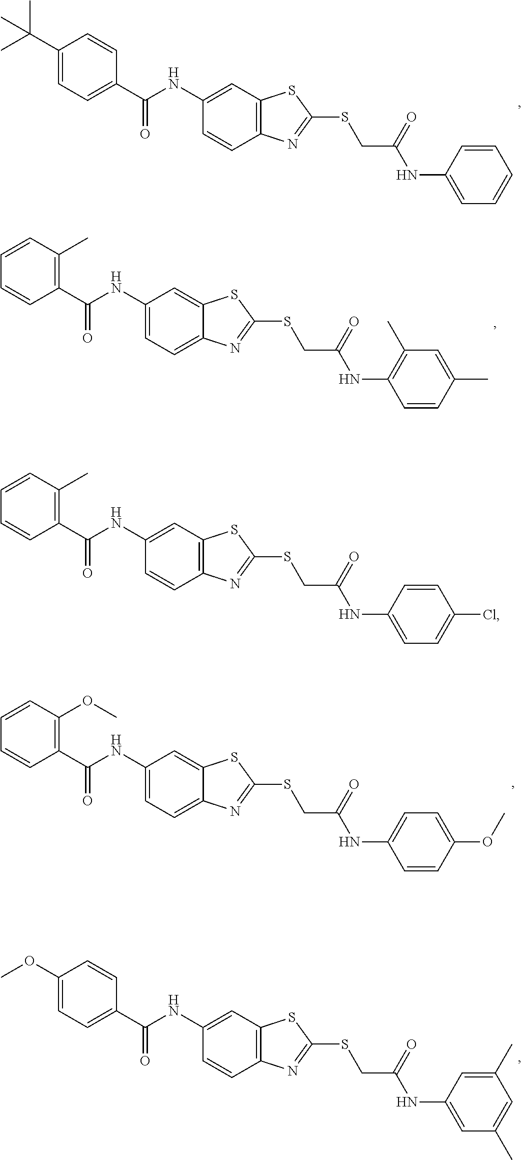 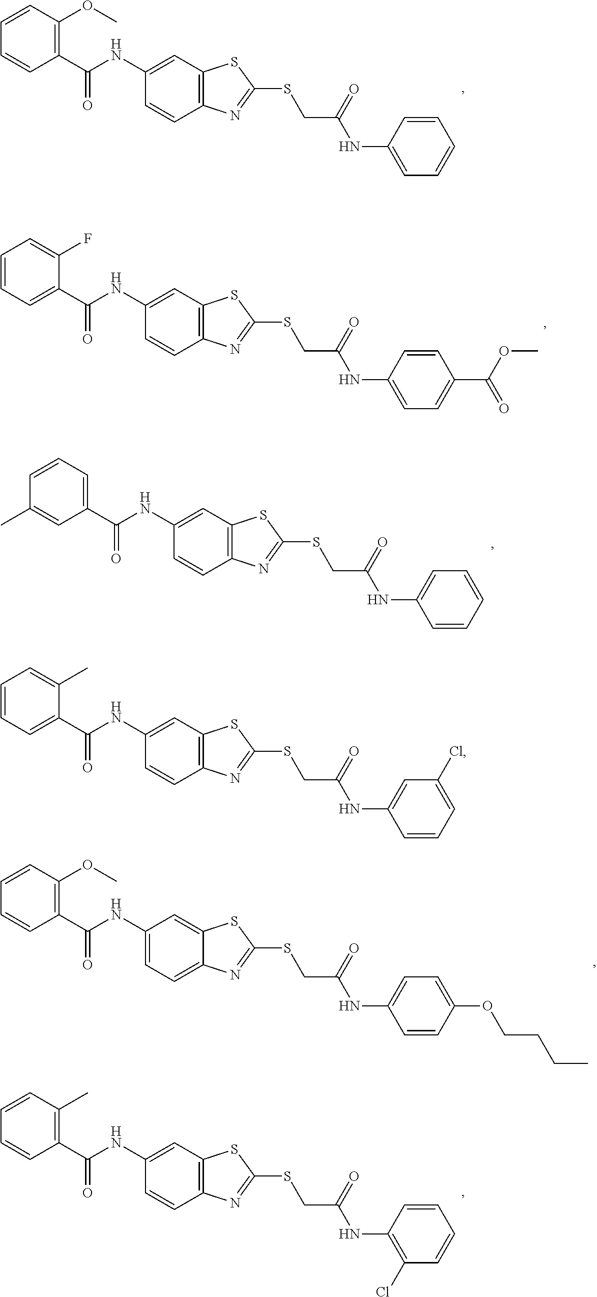 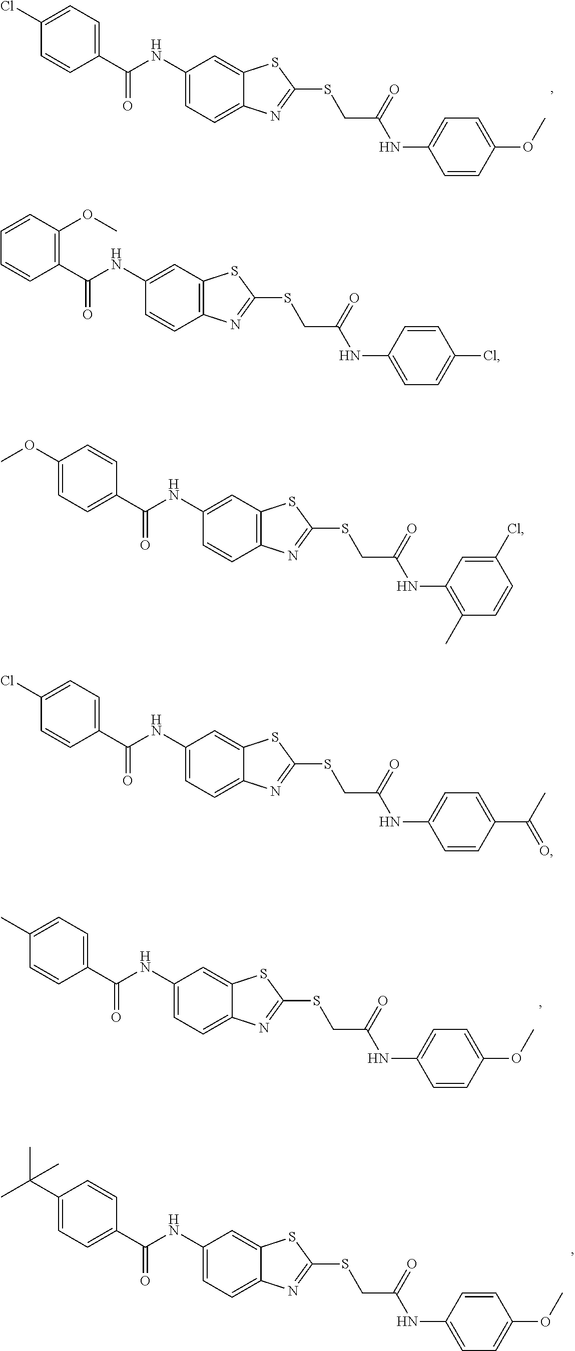 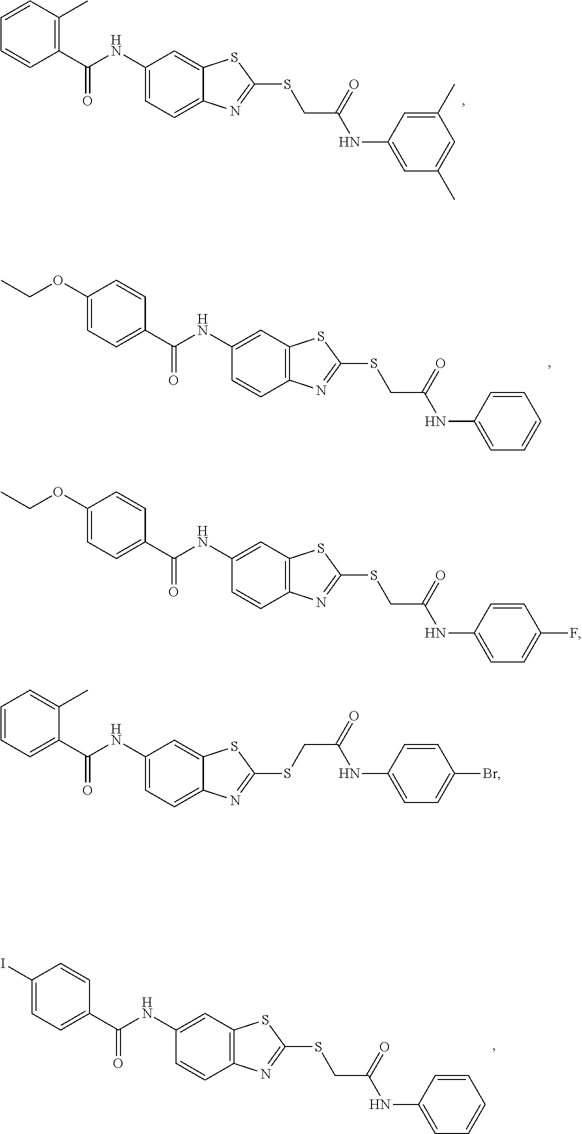 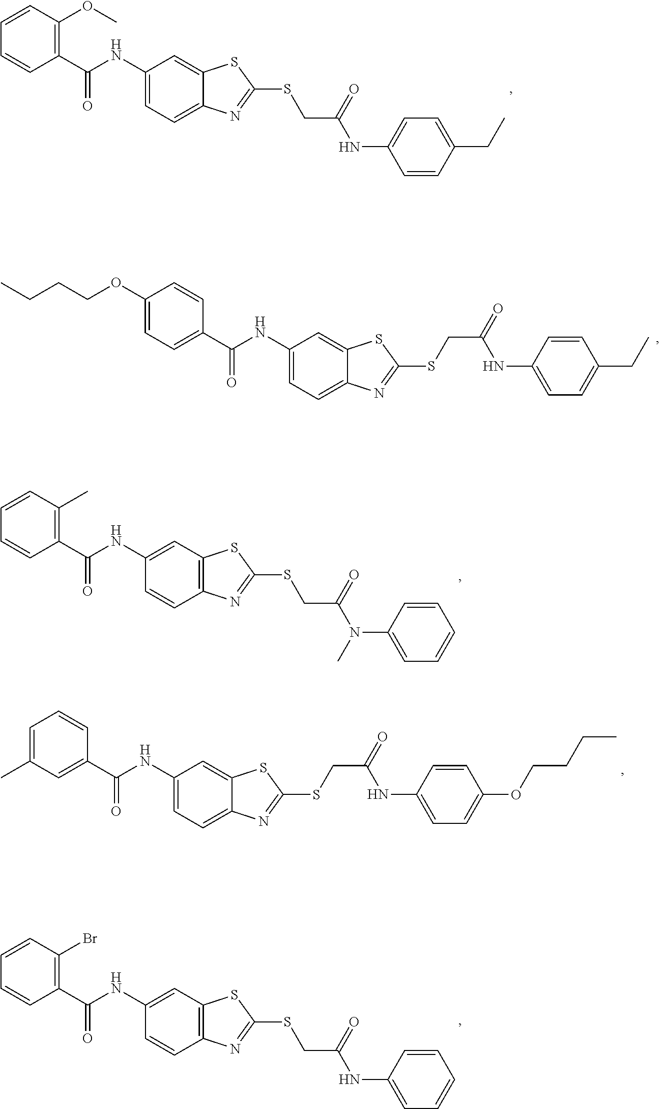 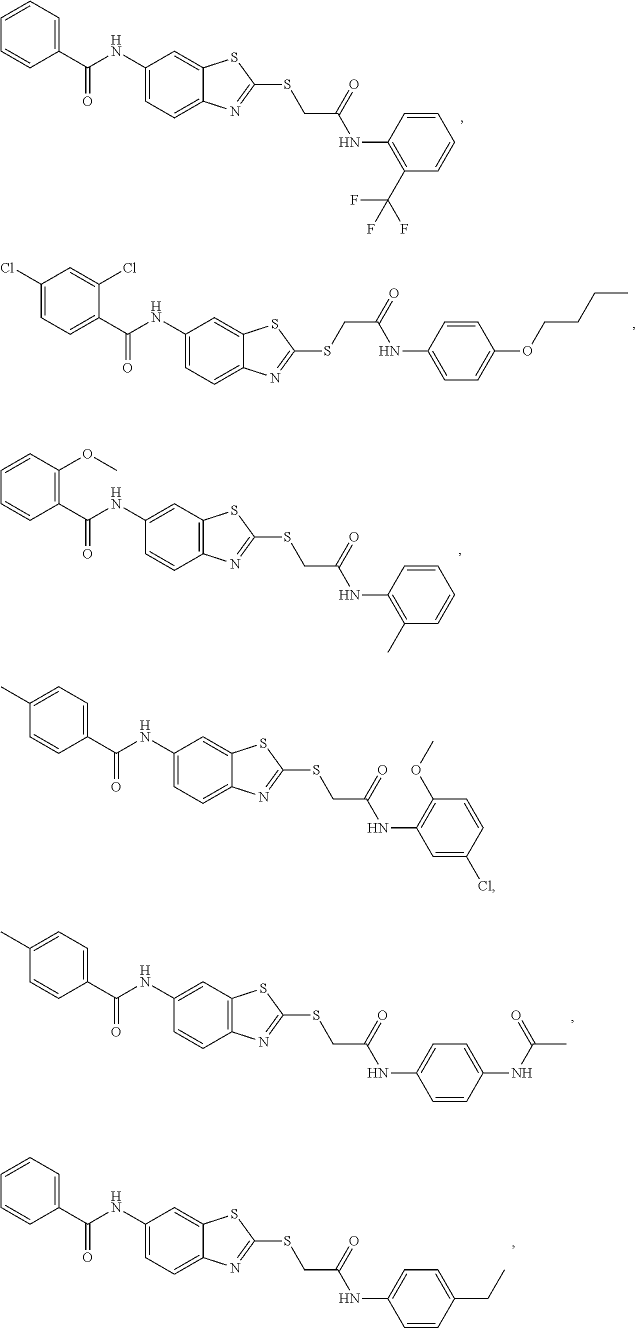 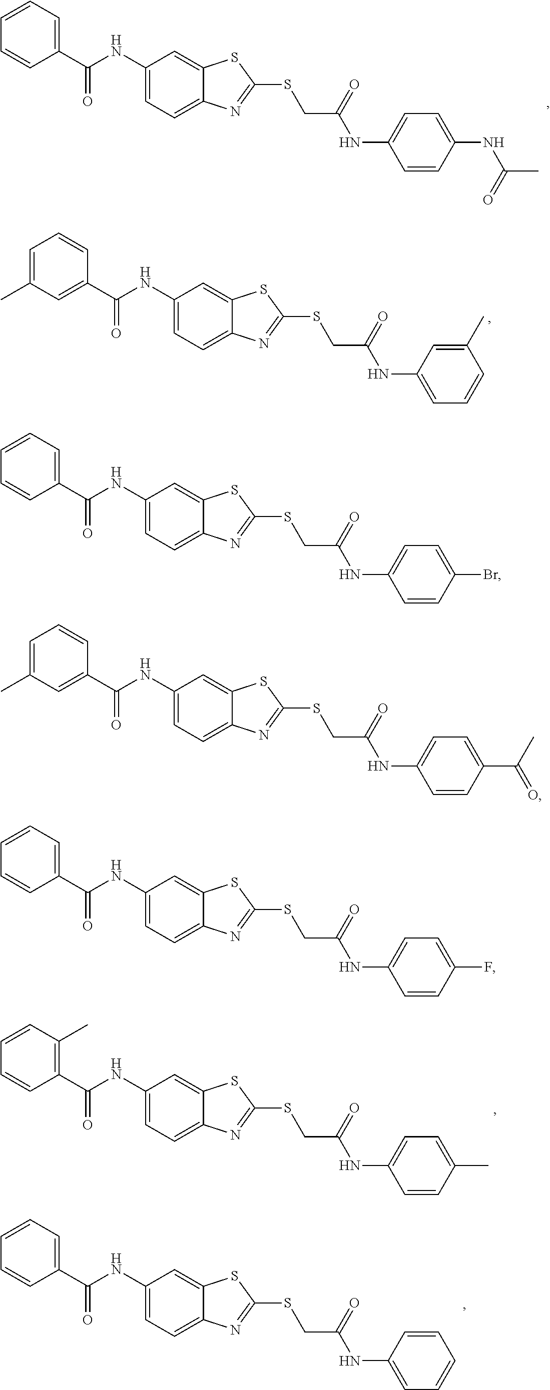 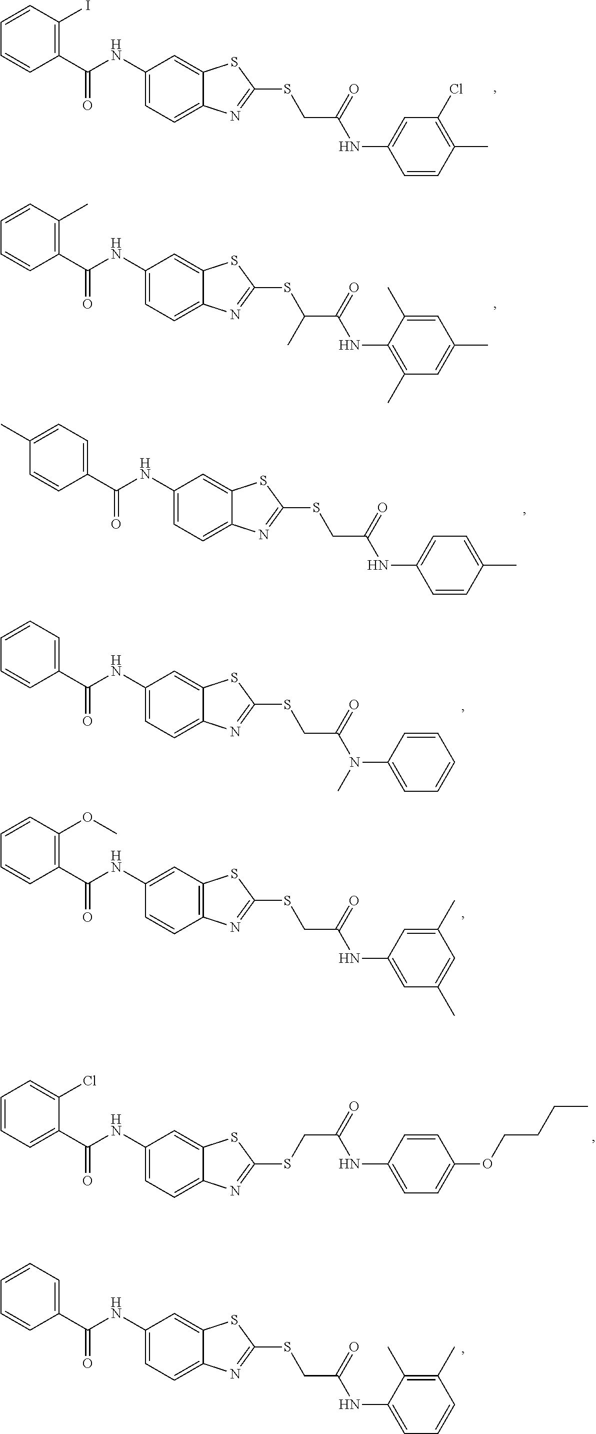 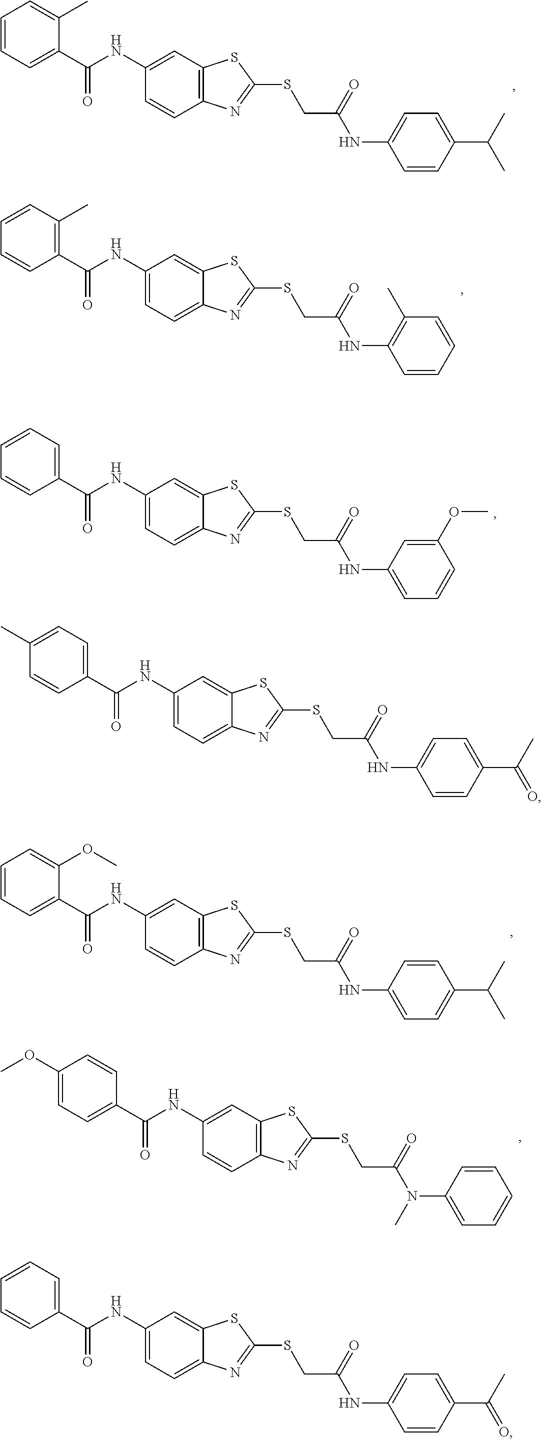 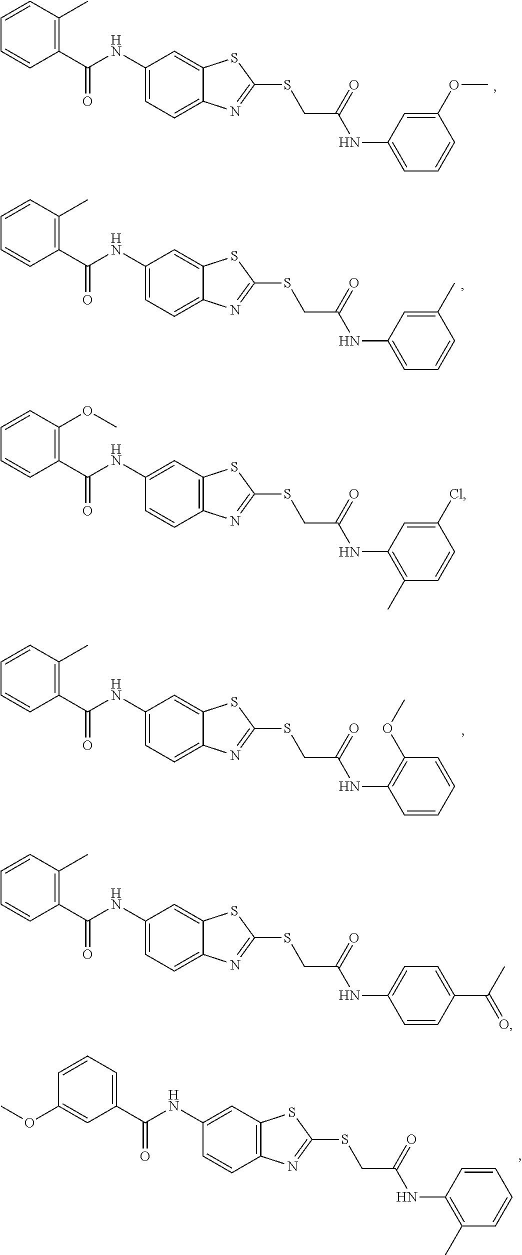 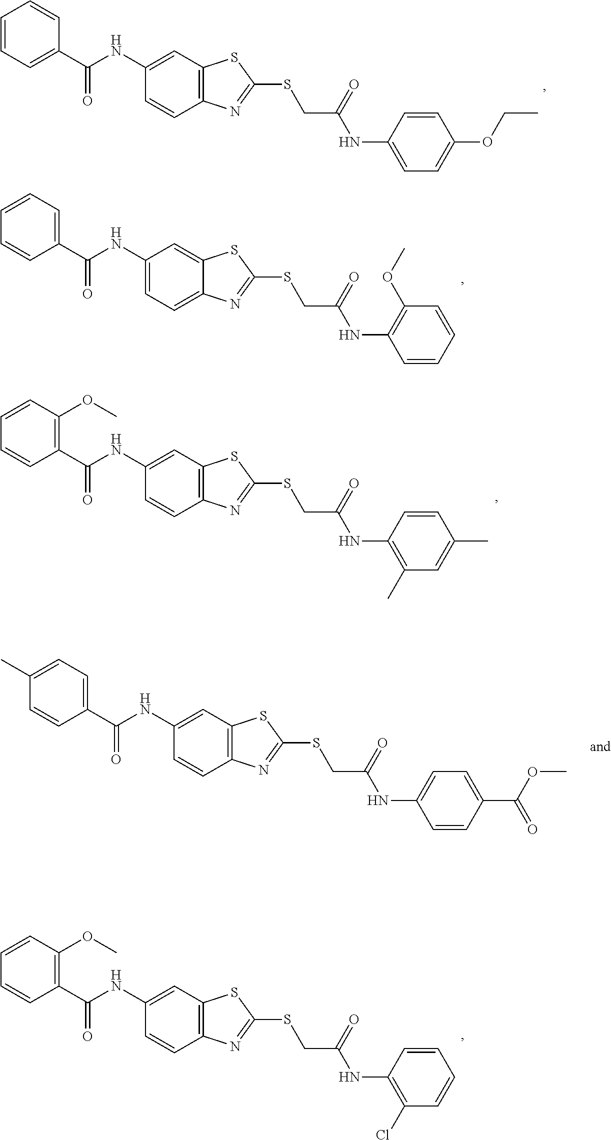
or a pharmaceutically acceptable salt of any one thereof.

35. The method of claim 27, where the compound of Formula (I) is represented by one of the following structures: 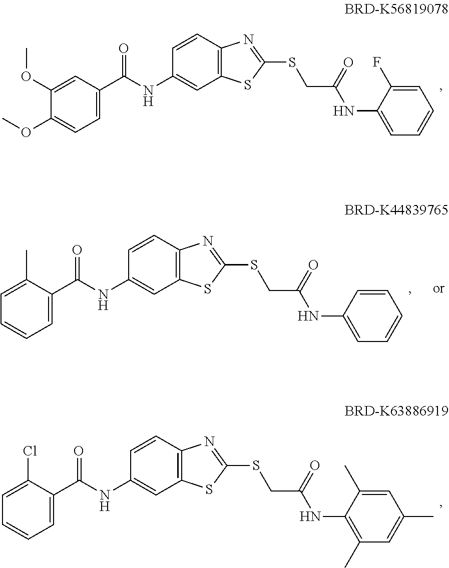 [SMILES: COc1ccc(C(=O)Nc2ccc3nc(SCC(=O)Nc4ccccc4F)sc3c2)cc1OC ; Cc1ccccc1C(=O)Nc1ccc2nc(SCC(=O)Nc3ccccc3)sc2c1 ; Cc1cc(C)c(NC(=O)CSc2nc3ccc(NC(=O)c4ccccc4Cl)cc3s2)c(C)c1]
or a pharmaceutically acceptable salt of any one thereof.

36. A method of treating a senescence-associated disease or disorder or an age-related disease or disorder in a subject comprising, administering to the subject a compound selected from: 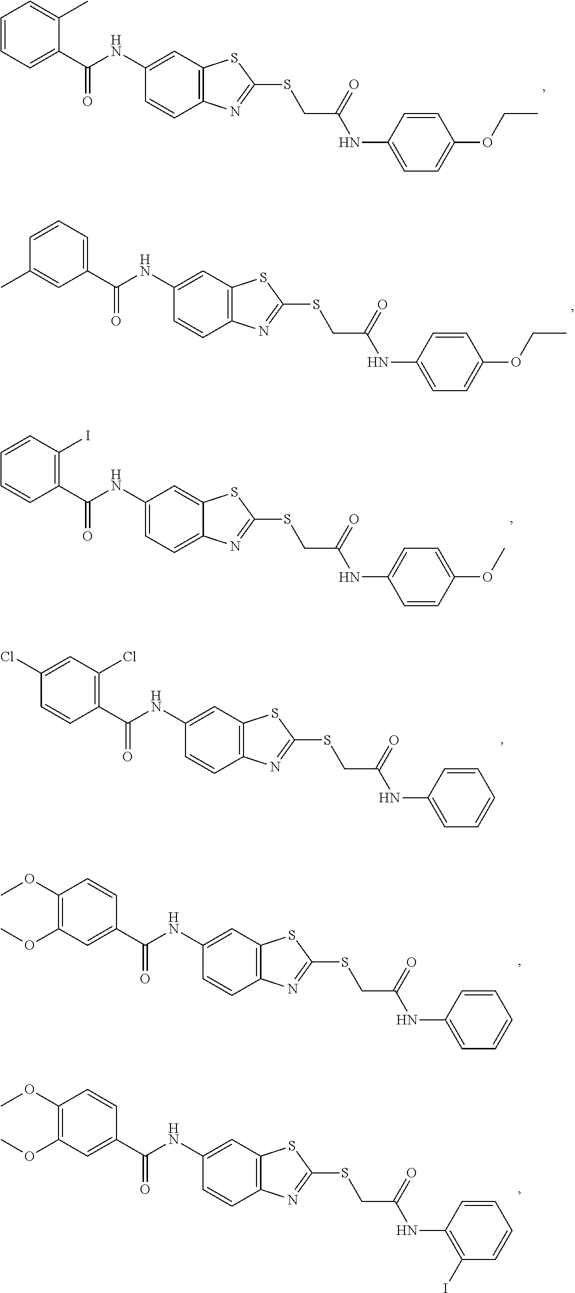 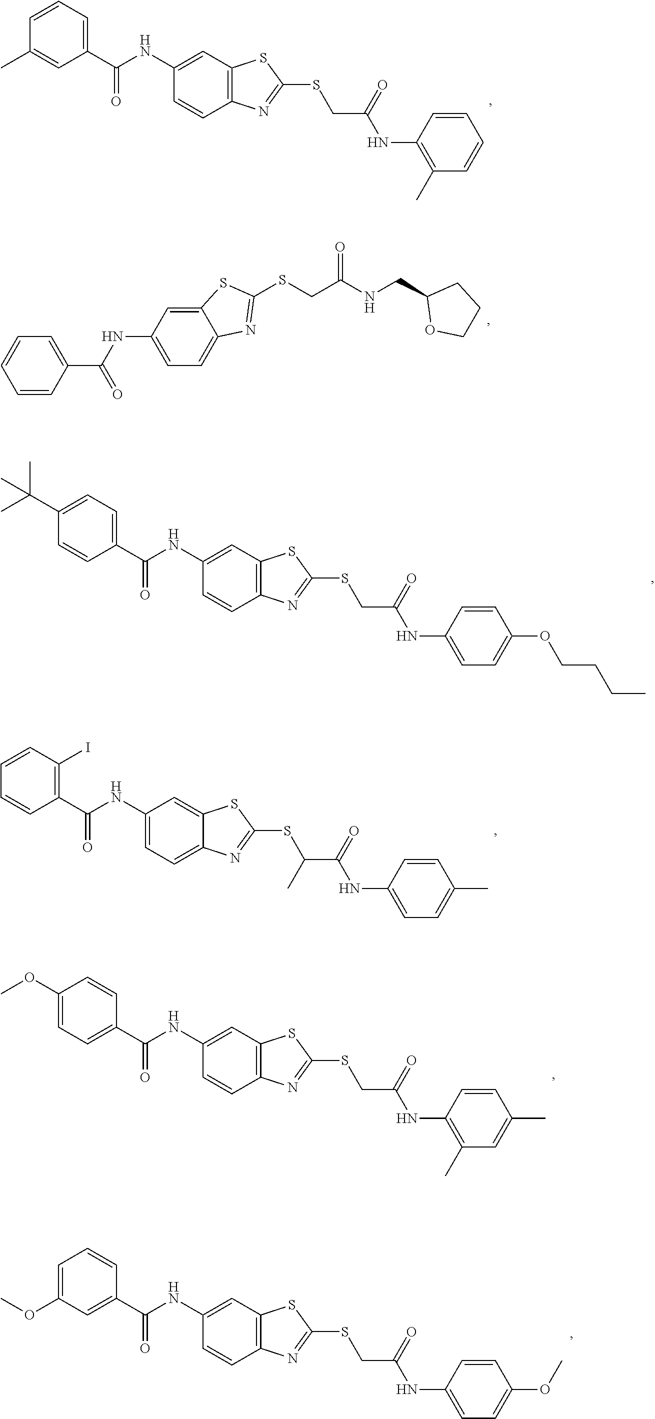 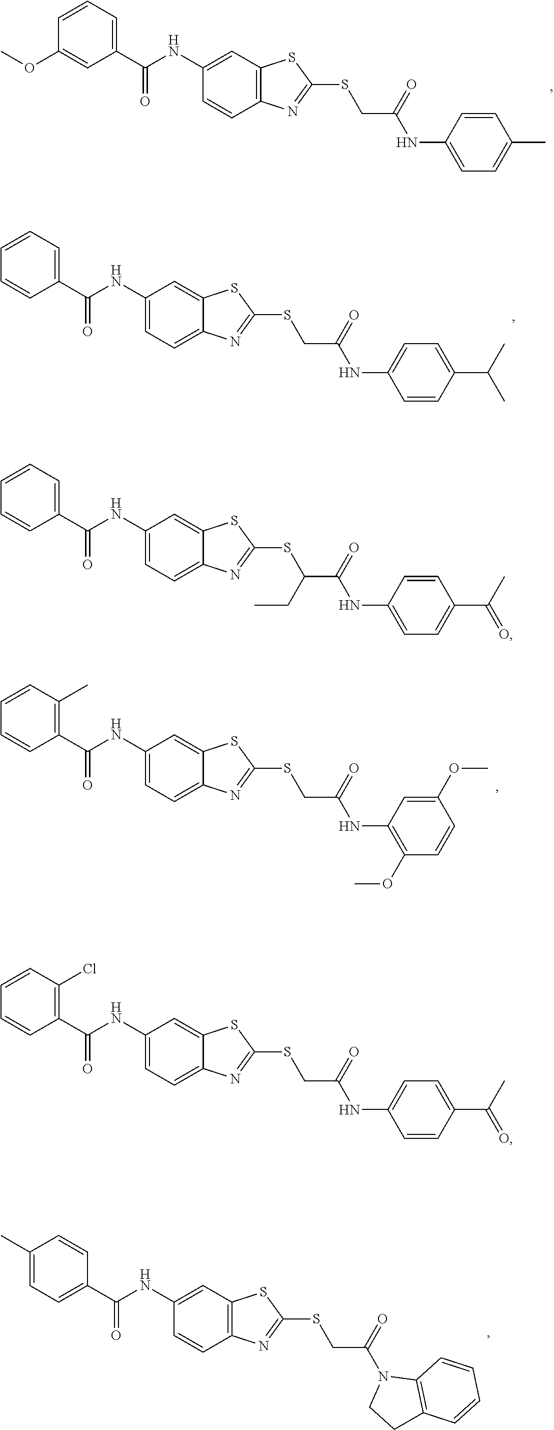 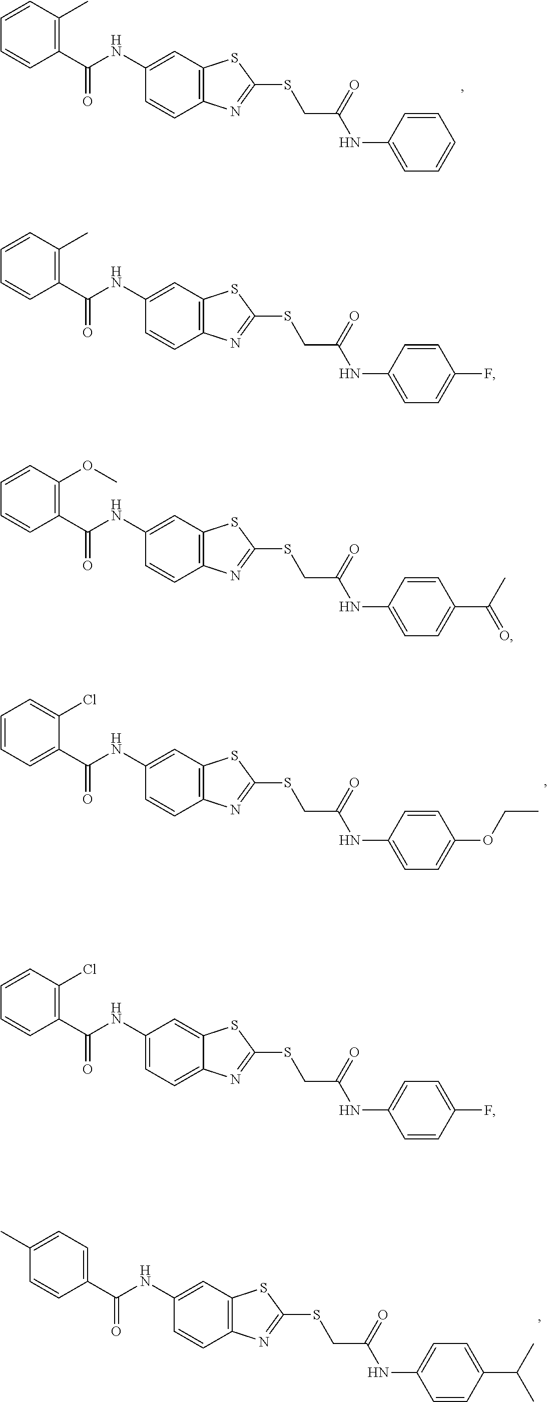 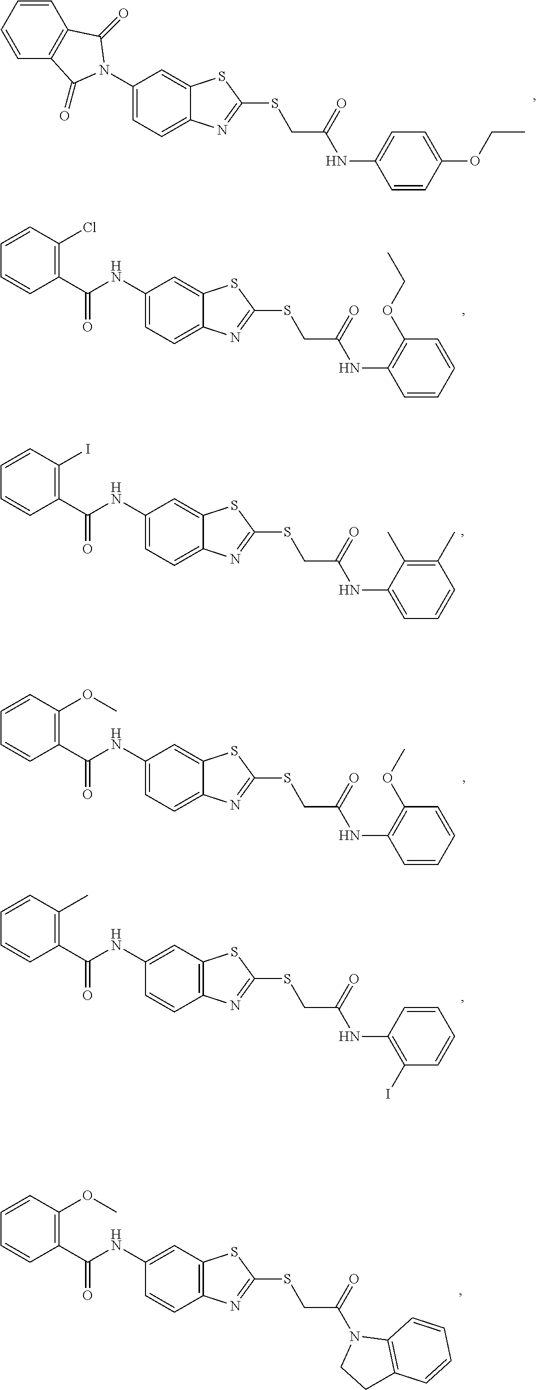 [SMILES: CCOc1ccc(NC(=O)CSc2nc3ccc(N4C(=O)c5ccccc5C4=O)cc3s2)cc1 ; CCOc1ccccc1NC(=O)CSc1nc2ccc(NC(=O)c3ccccc3Cl)cc2s1 ; Cc1cccc(NC(=O)CSc2nc3ccc(NC(=O)c4ccccc4I)cc3s2)c1C ; COc1ccccc1NC(=O)CSc1nc2ccc(NC(=O)c3ccccc3OC)cc2s1 ; Cc1ccccc1C(=O)Nc1ccc2nc(SCC(=O)Nc3ccccc3I)sc2c1 ; COc1ccccc1C(=O)Nc1ccc2nc(SCC(=O)N3CCc4ccccc43)sc2c1] 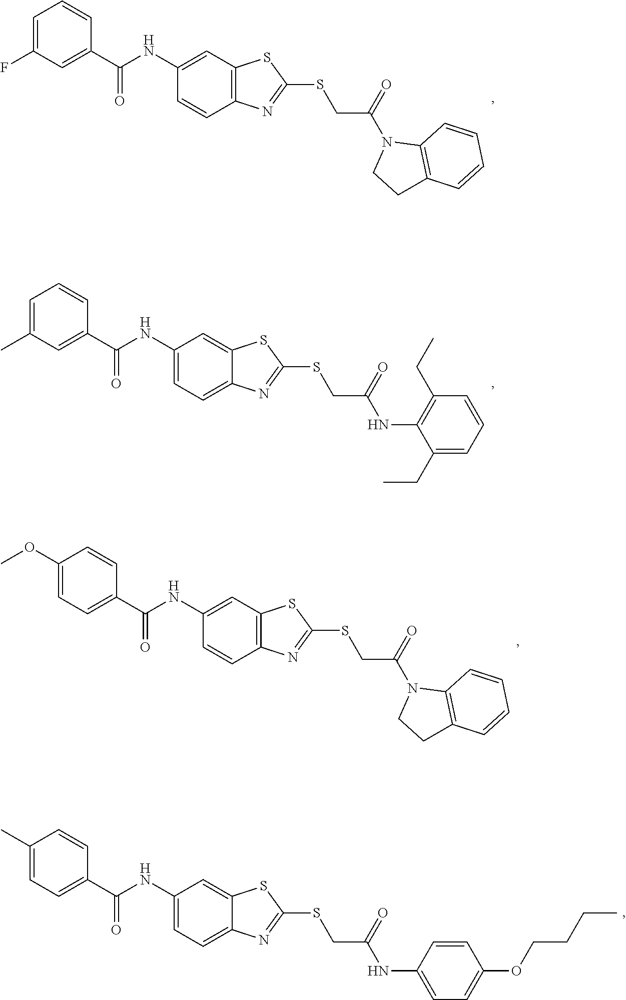 [SMILES: O=C(Nc1ccc2nc(SCC(=O)N3CCc4ccccc43)sc2c1)c1cccc(F)c1 ; CCc1cccc(CC)c1NC(=O)CSc1nc2ccc(NC(=O)c3cccc(C)c3)cc2s1 ; COc1ccc(C(=O)Nc2ccc3nc(SCC(=O)N4CCc5ccccc54)sc3c2)cc1 ; CCCCOc1ccc(NC(=O)CSc2nc3ccc(NC(=O)c4ccc(C)cc4)cc3s2)cc1] 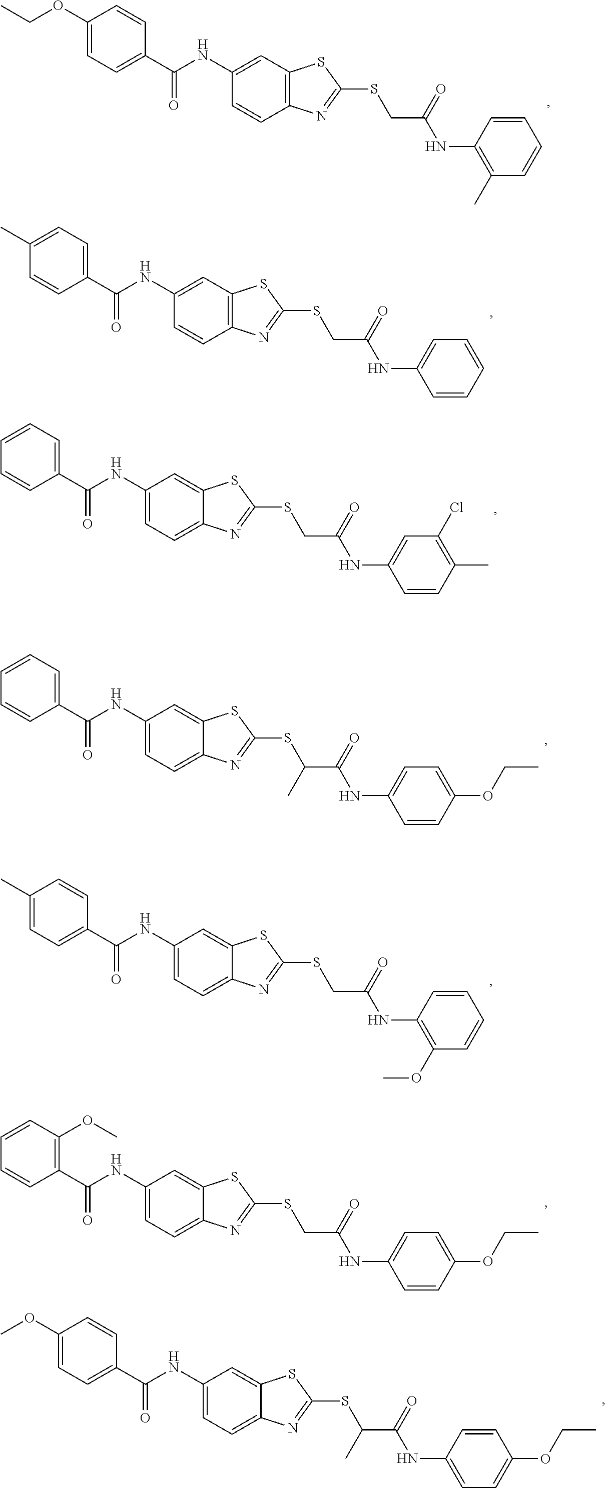 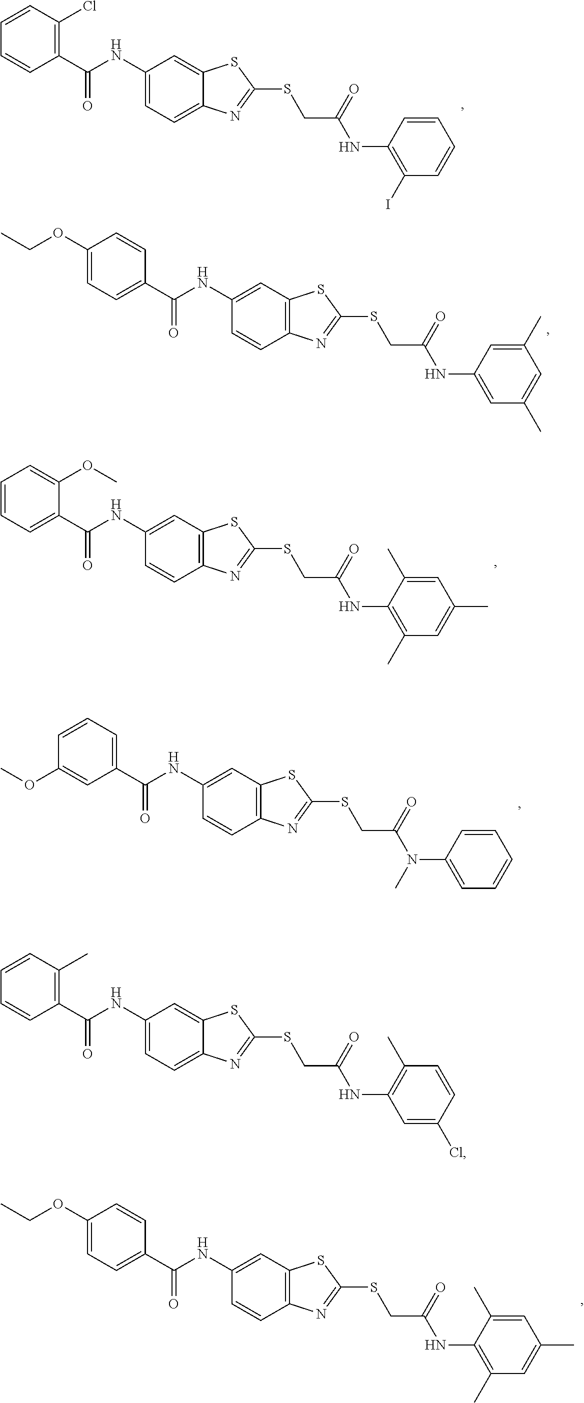 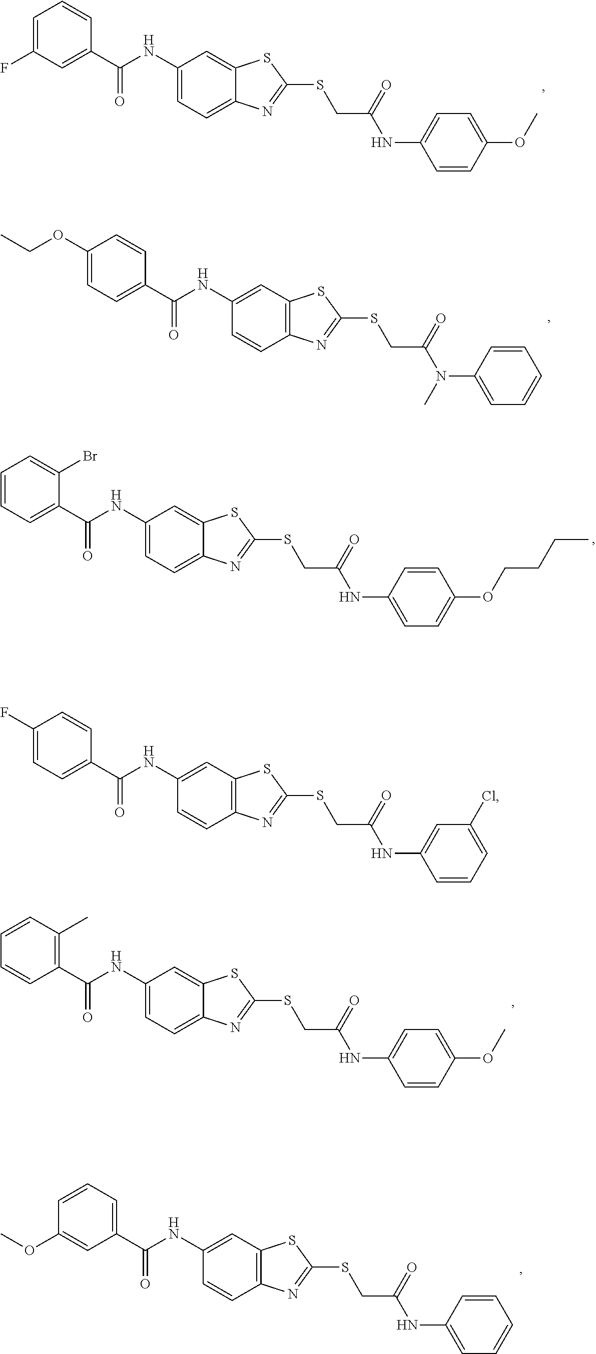 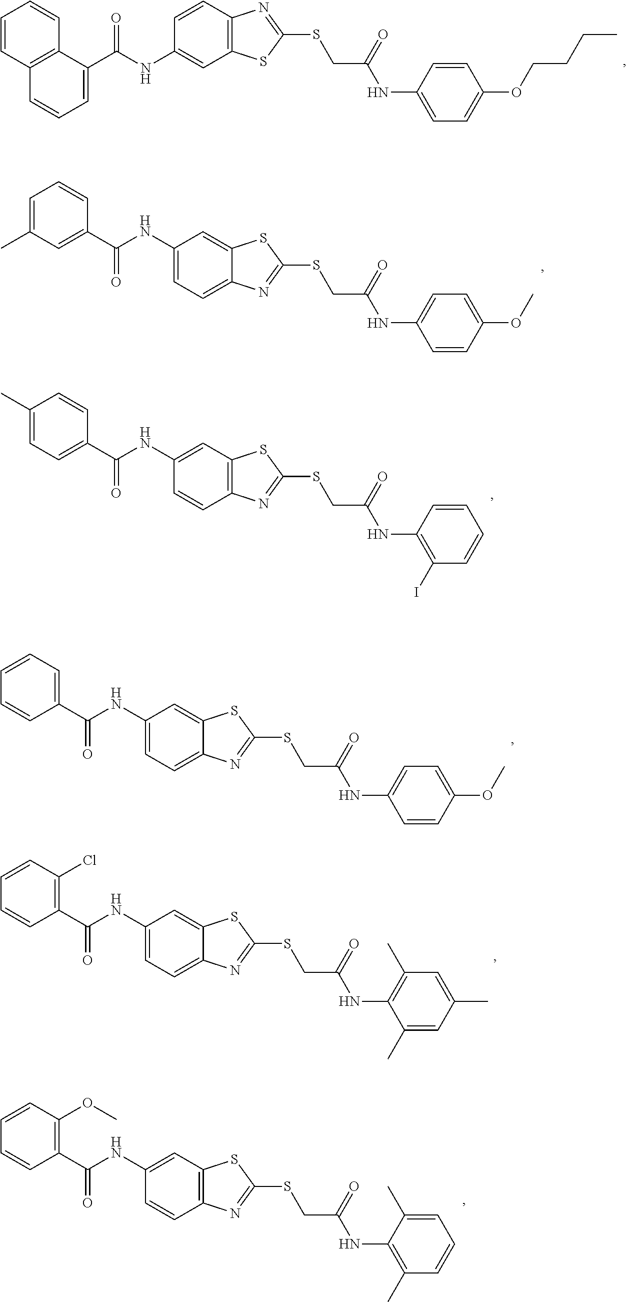 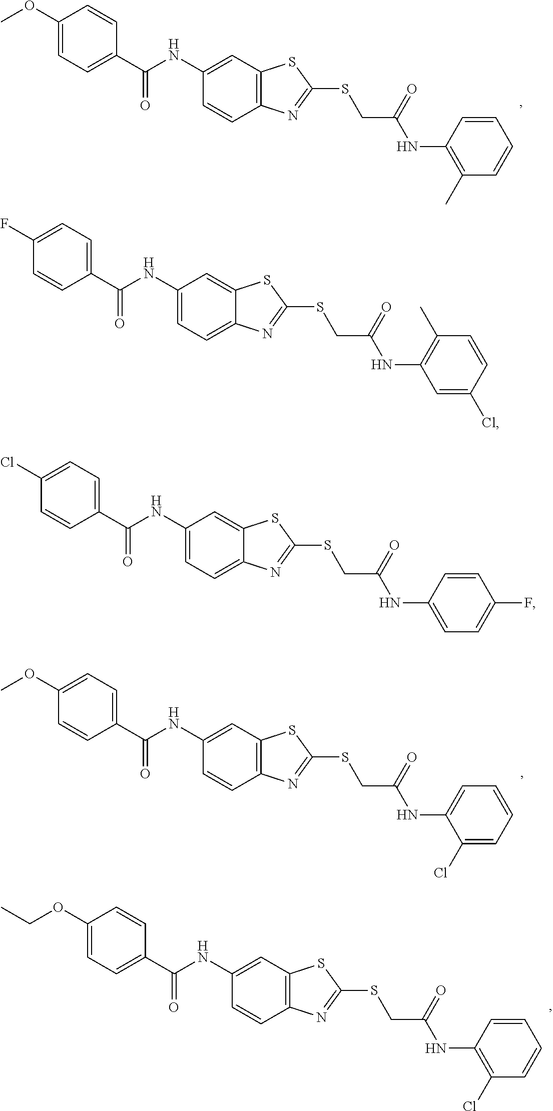 [SMILES: COc1ccc(C(=O)Nc2ccc3nc(SCC(=O)Nc4ccccc4C)sc3c2)cc1 ; Cc1ccc(Cl)cc1NC(=O)CSc1nc2ccc(NC(=O)c3ccc(F)cc3)cc2s1 ; O=C(CSc1nc2ccc(NC(=O)c3ccc(Cl)cc3)cc2s1)Nc1ccc(F)cc1 ; COc1ccc(C(=O)Nc2ccc3nc(SCC(=O)Nc4ccccc4Cl)sc3c2)cc1 ; CCOc1ccc(C(=O)Nc2ccc3nc(SCC(=O)Nc4ccccc4Cl)sc3c2)cc1] 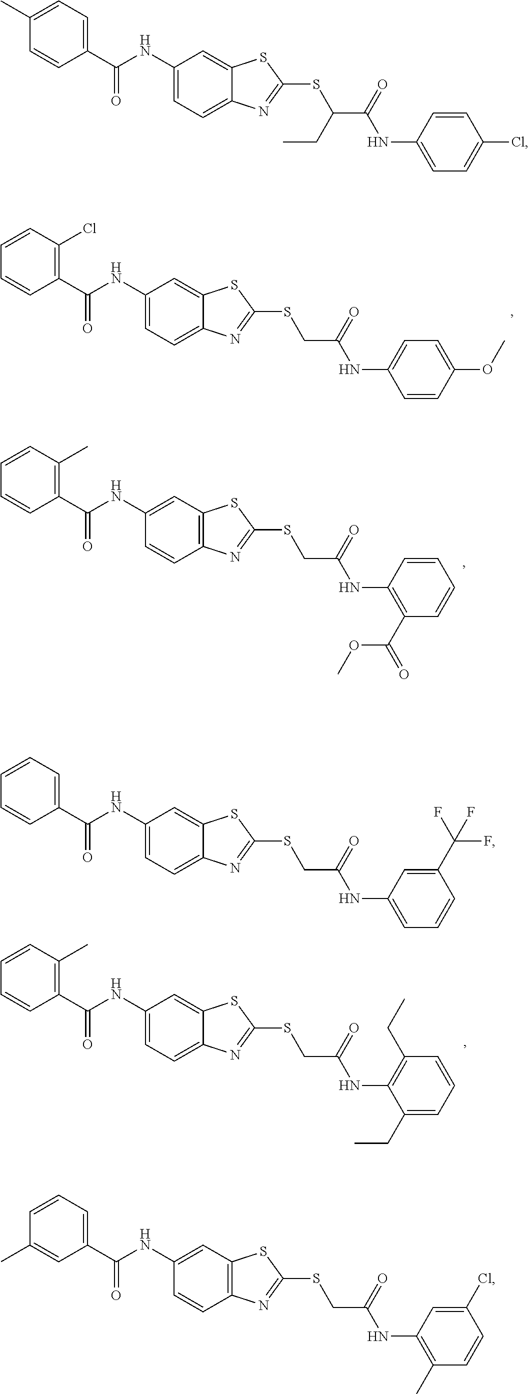 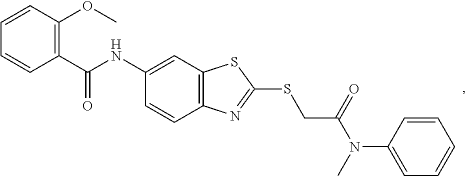 [SMILES: COc1ccccc1C(=O)Nc1ccc2nc(SCC(=O)N(C)c3ccccc3)sc2c1] 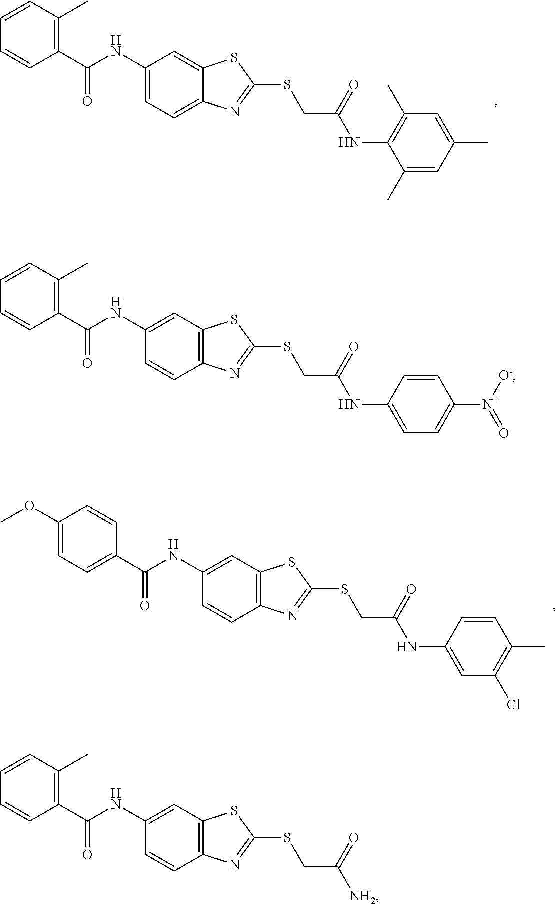 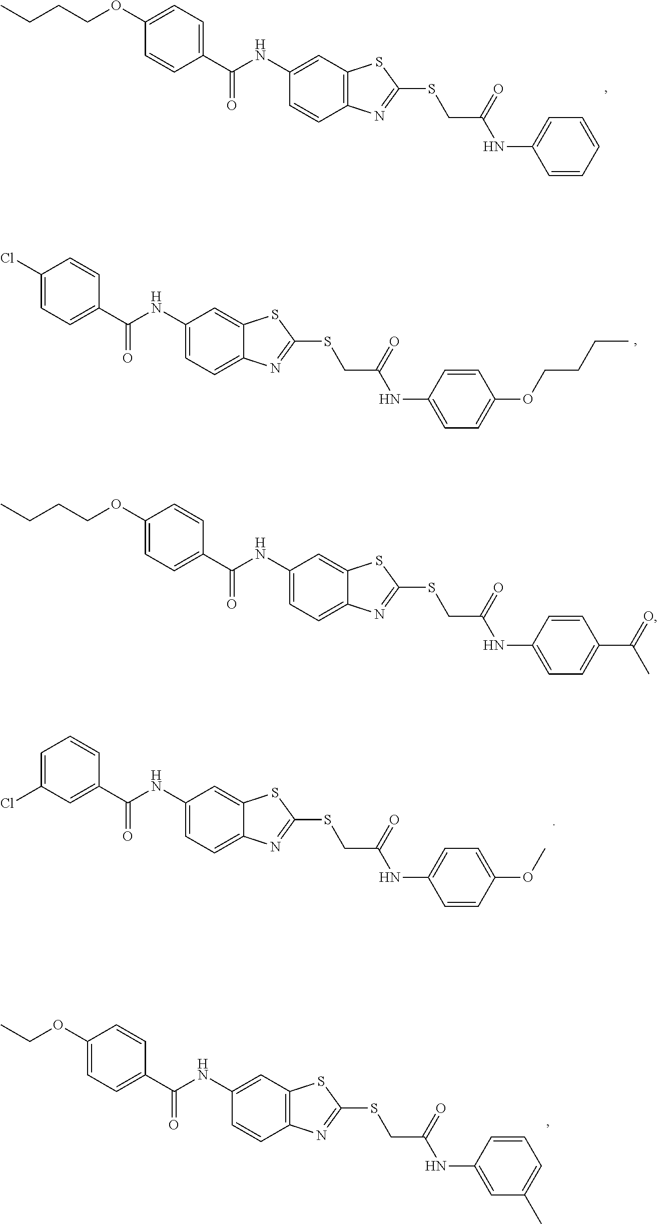 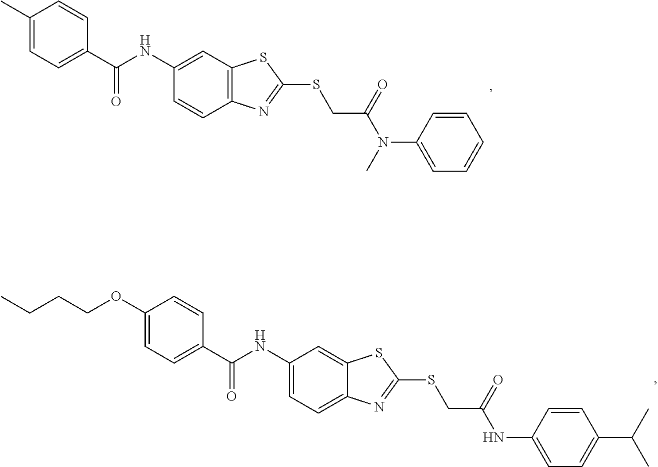 [SMILES: Cc1ccc(C(=O)Nc2ccc3nc(SCC(=O)N(C)c4ccccc4)sc3c2)cc1 ; CCCCOc1ccc(C(=O)Nc2ccc3nc(SCC(=O)Nc4ccc(C(C)C)cc4)sc3c2)cc1] 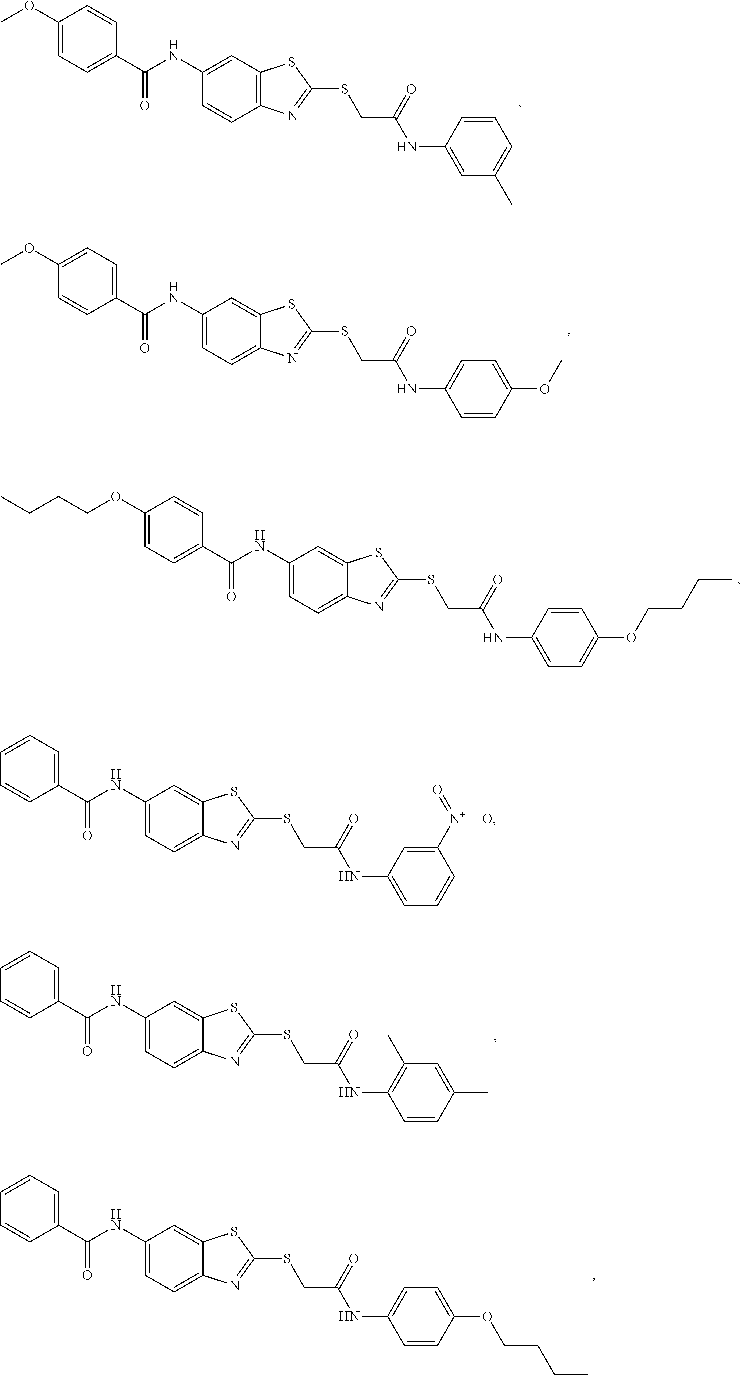 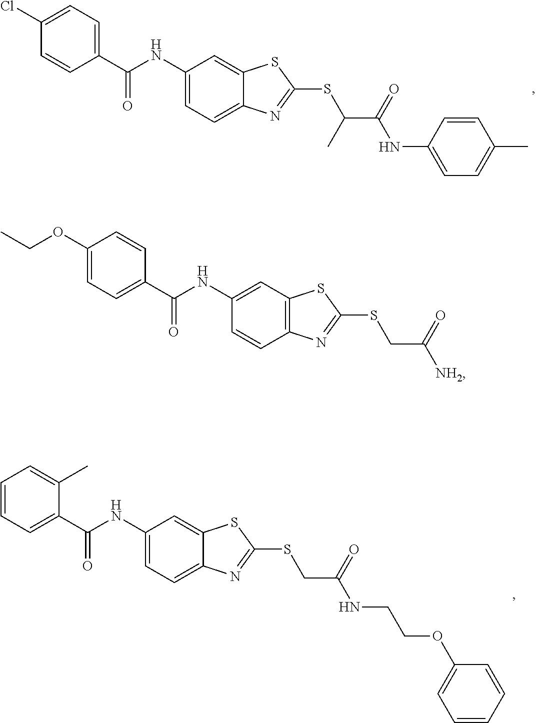 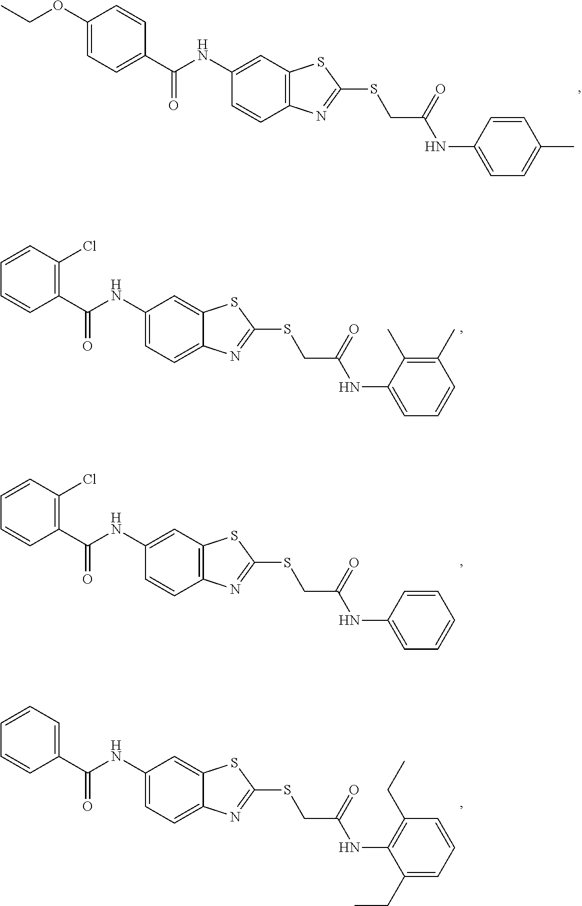 [SMILES: CCOc1ccc(C(=O)Nc2ccc3nc(SCC(=O)Nc4ccc(C)cc4)sc3c2)cc1 ; Cc1cccc(NC(=O)CSc2nc3ccc(NC(=O)c4ccccc4Cl)cc3s2)c1C ; O=C(CSc1nc2ccc(NC(=O)c3ccccc3Cl)cc2s1)Nc1ccccc1 ; CCc1cccc(CC)c1NC(=O)CSc1nc2ccc(NC(=O)c3ccccc3)cc2s1] 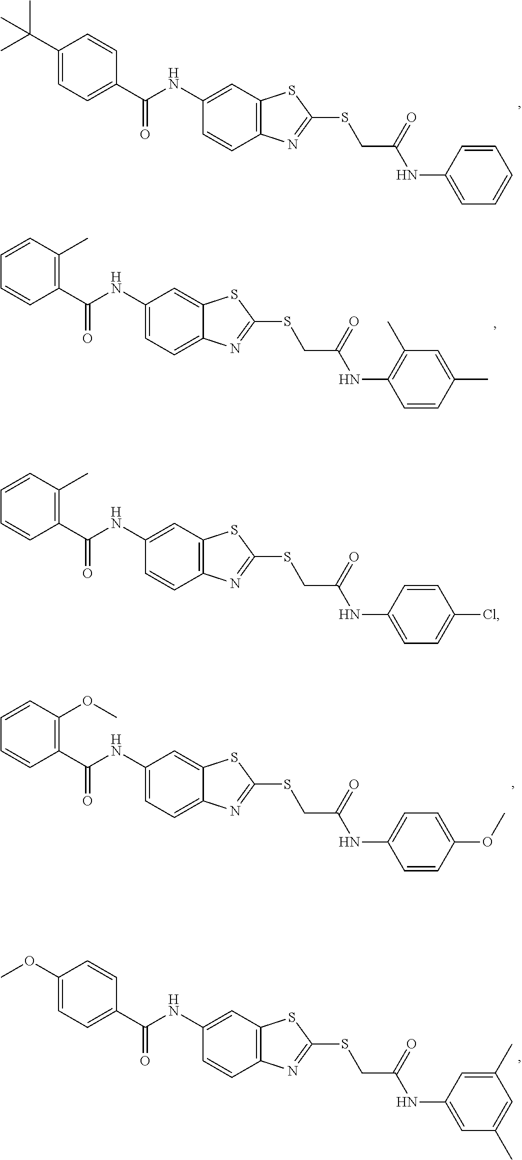 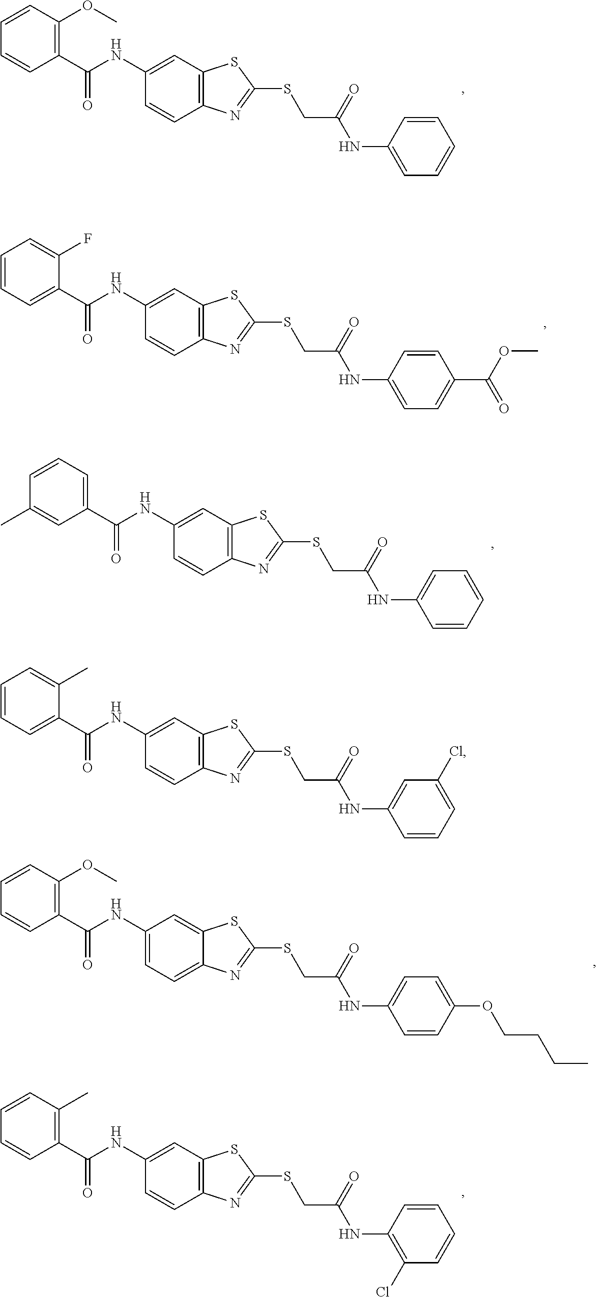 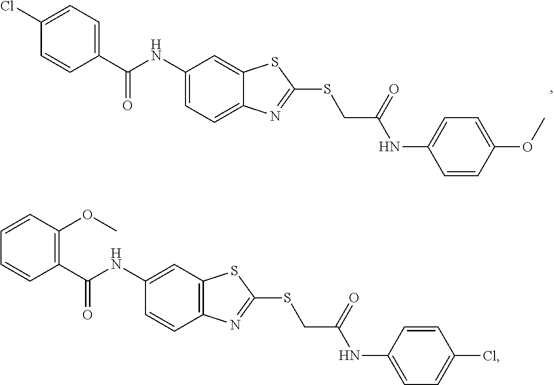 [SMILES: COc1ccc(NC(=O)CSc2nc3ccc(NC(=O)c4ccc(Cl)cc4)cc3s2)cc1 ; COc1ccccc1C(=O)Nc1ccc2nc(SCC(=O)Nc3ccc(Cl)cc3)sc2c1] 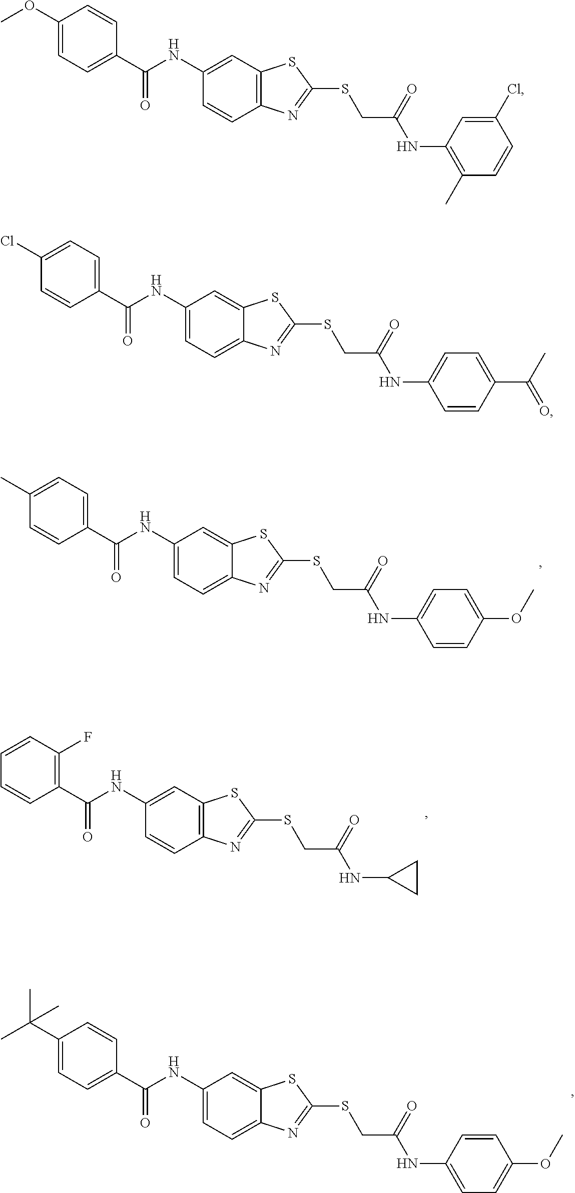 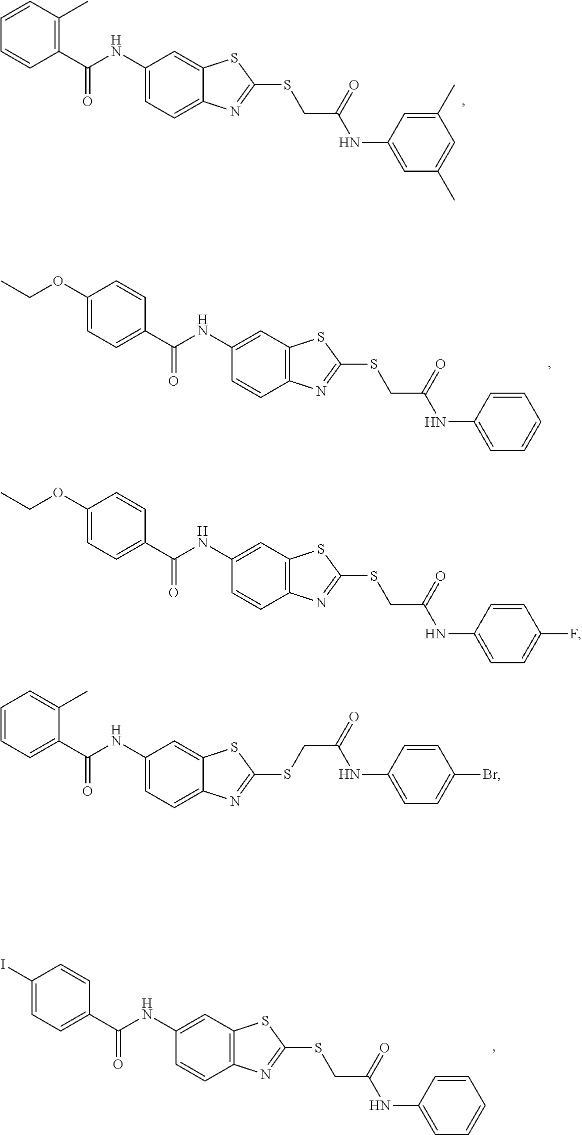 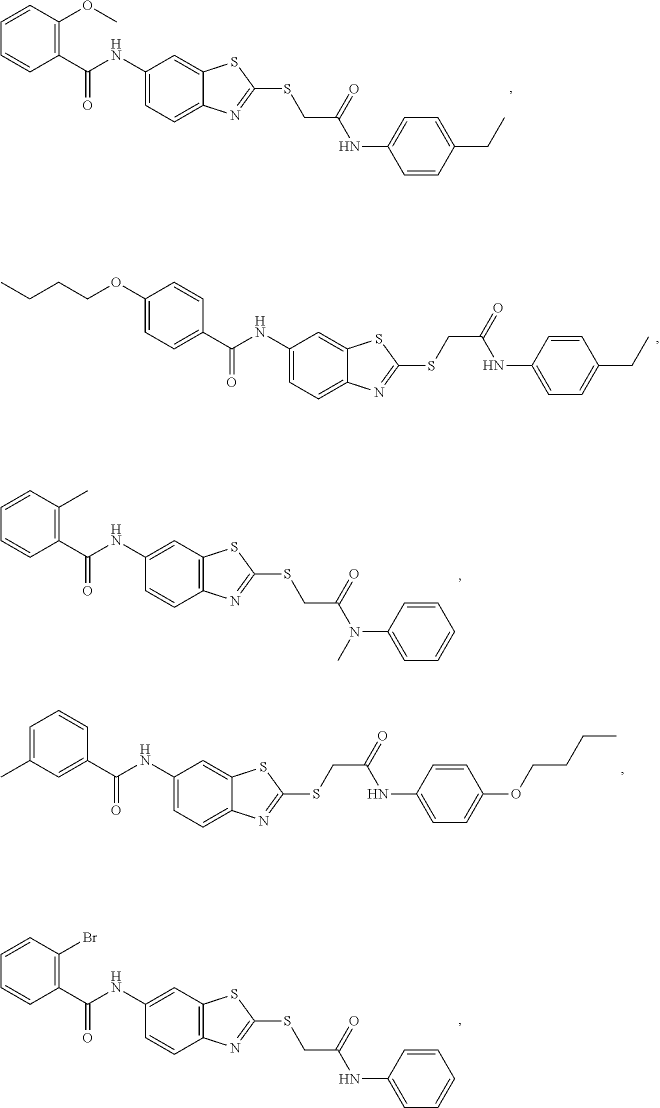 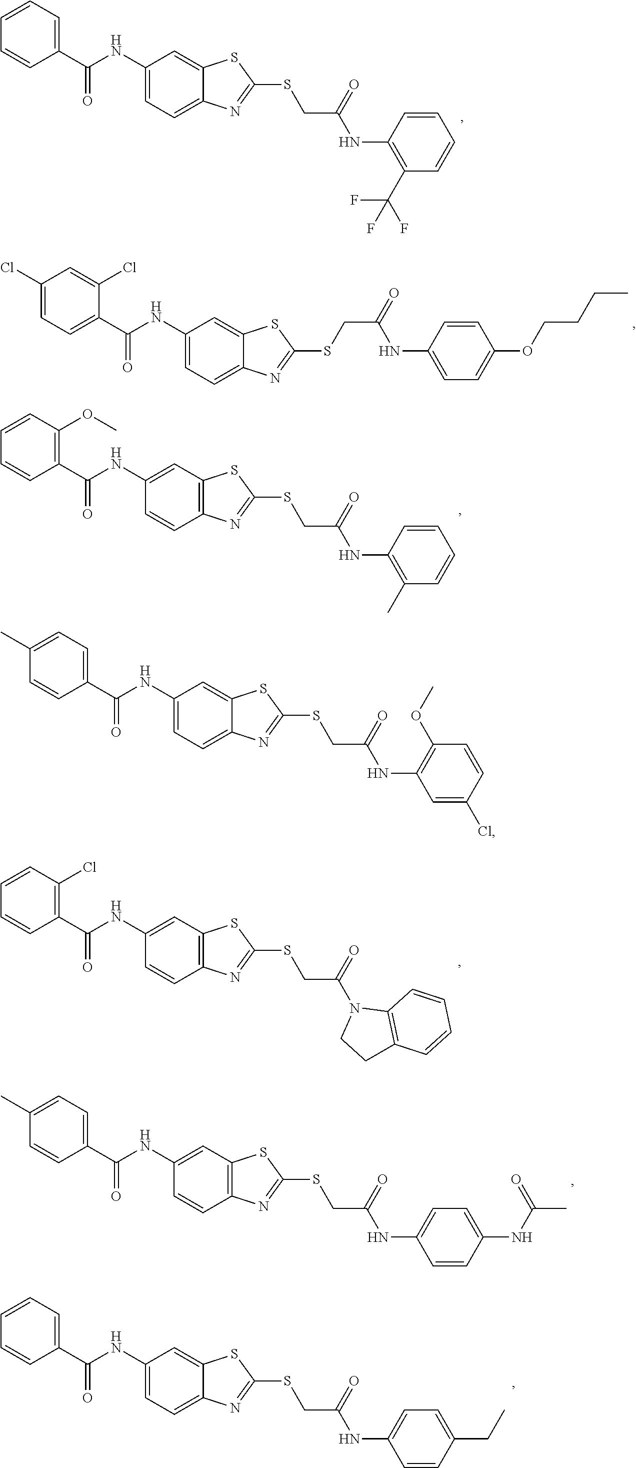 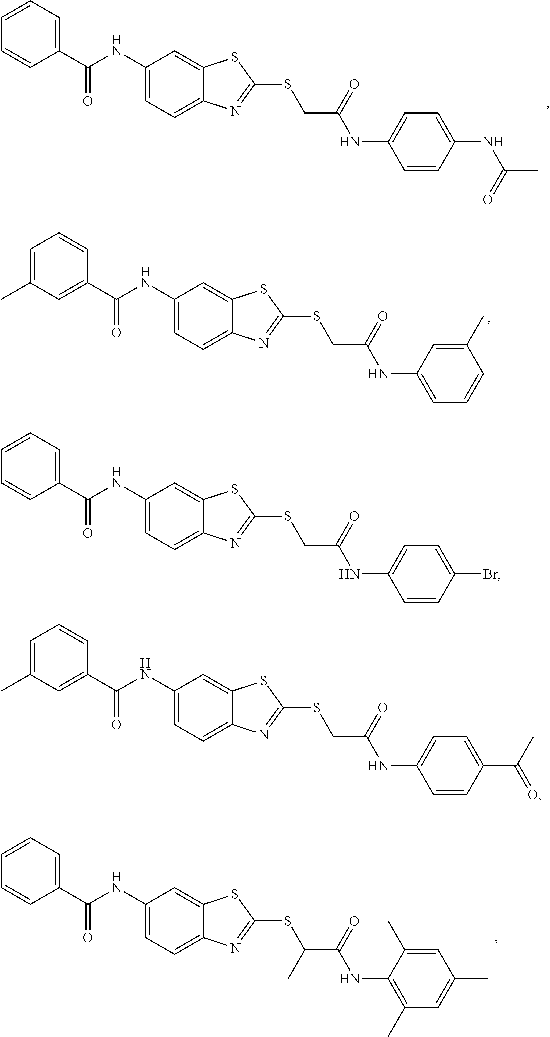 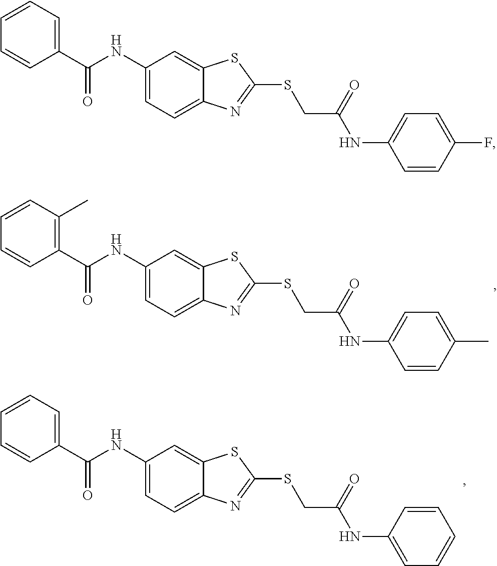 [SMILES: O=C(CSc1nc2ccc(NC(=O)c3ccccc3)cc2s1)Nc1ccc(F)cc1 ; Cc1ccc(NC(=O)CSc2nc3ccc(NC(=O)c4ccccc4C)cc3s2)cc1 ; O=C(CSc1nc2ccc(NC(=O)c3ccccc3)cc2s1)Nc1ccccc1] 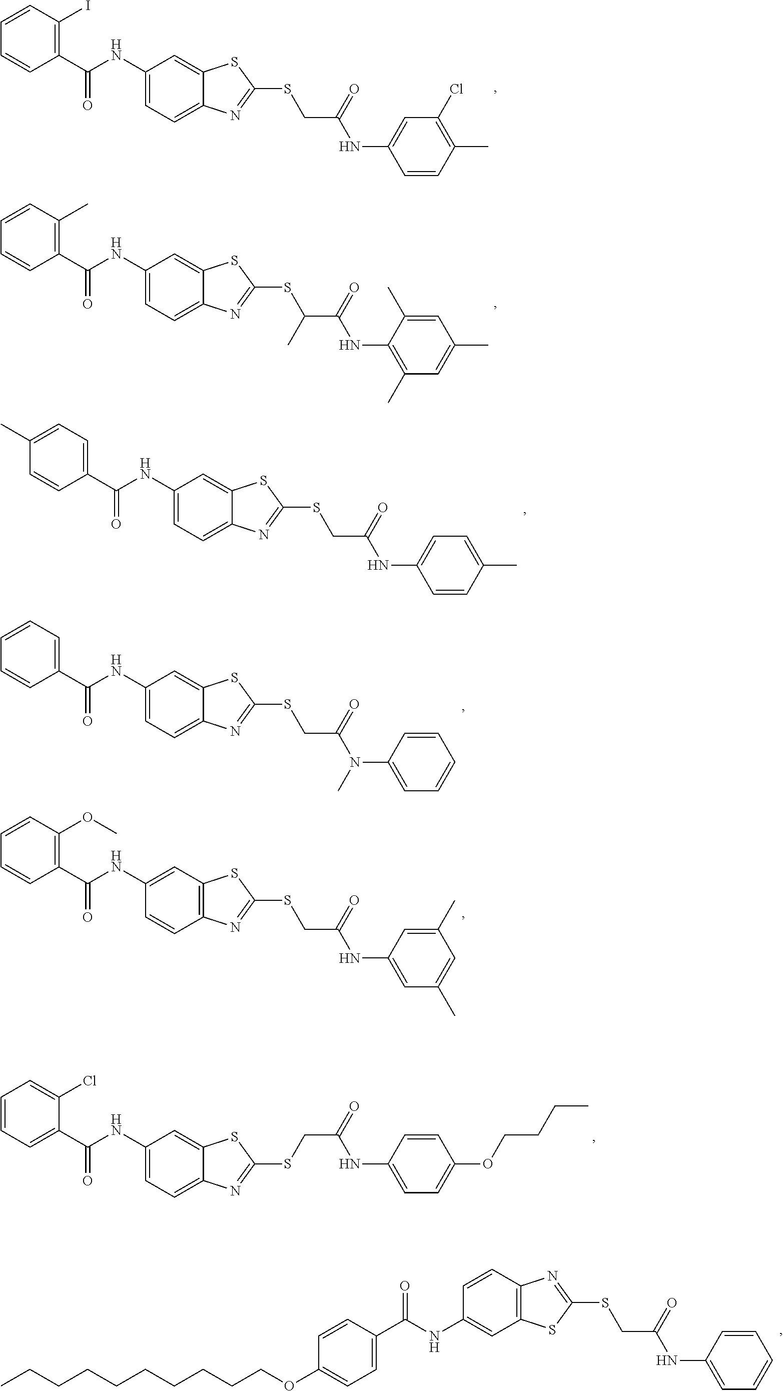 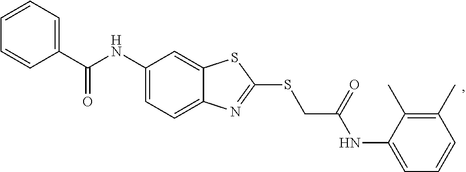 [SMILES: Cc1cccc(NC(=O)CSc2nc3ccc(NC(=O)c4ccccc4)cc3s2)c1C] 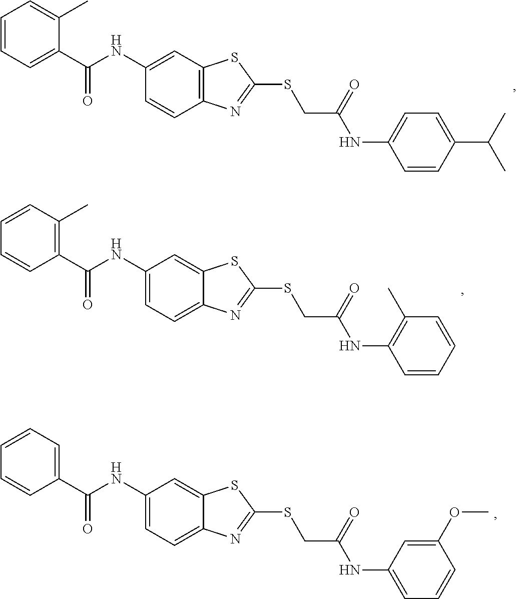 [SMILES: Cc1ccccc1C(=O)Nc1ccc2nc(SCC(=O)Nc3ccc(C(C)C)cc3)sc2c1 ; Cc1ccccc1NC(=O)CSc1nc2ccc(NC(=O)c3ccccc3C)cc2s1 ; COc1cccc(NC(=O)CSc2nc3ccc(NC(=O)c4ccccc4)cc3s2)c1] 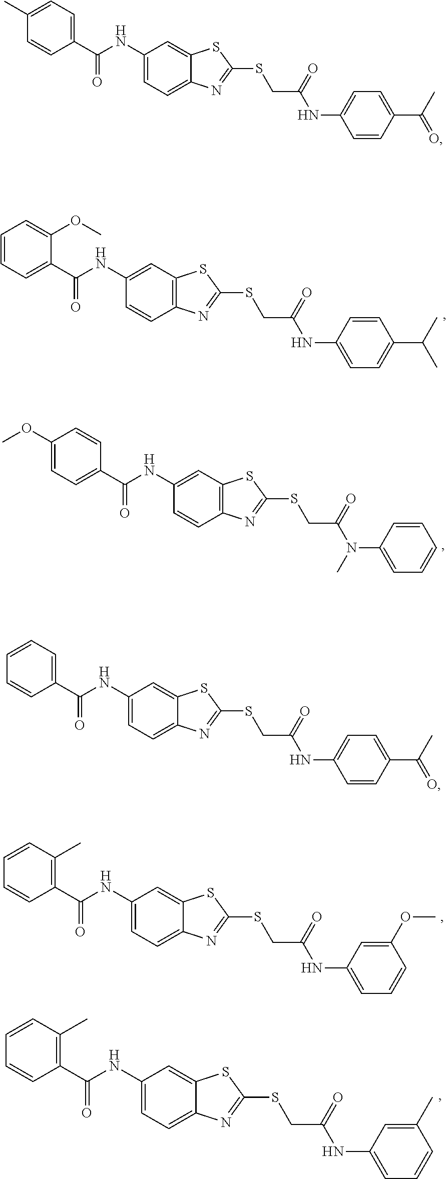 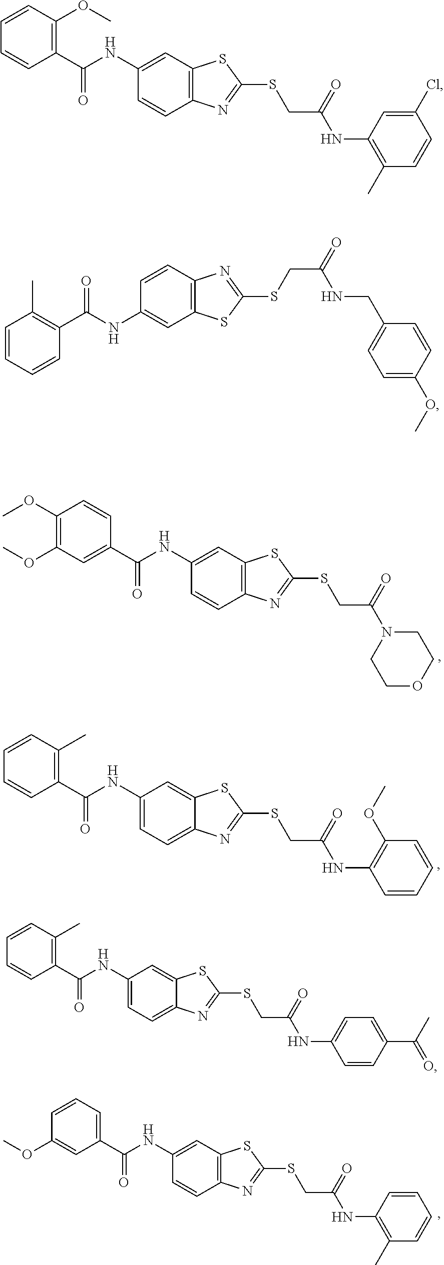 [SMILES: COc1ccccc1C(=O)Nc1ccc2nc(SCC(=O)Nc3cc(Cl)ccc3C)sc2c1 ; COc1ccc(CNC(=O)CSc2nc3ccc(NC(=O)c4ccccc4C)cc3s2)cc1 ; COc1ccc(C(=O)Nc2ccc3nc(SCC(=O)N4CCOCC4)sc3c2)cc1OC ; COc1ccccc1NC(=O)CSc1nc2ccc(NC(=O)c3ccccc3C)cc2s1 ; CC(=O)c1ccc(NC(=O)CSc2nc3ccc(NC(=O)c4ccccc4C)cc3s2)cc1 ; COc1cccc(C(=O)Nc2ccc3nc(SCC(=O)Nc4ccccc4C)sc3c2)c1] 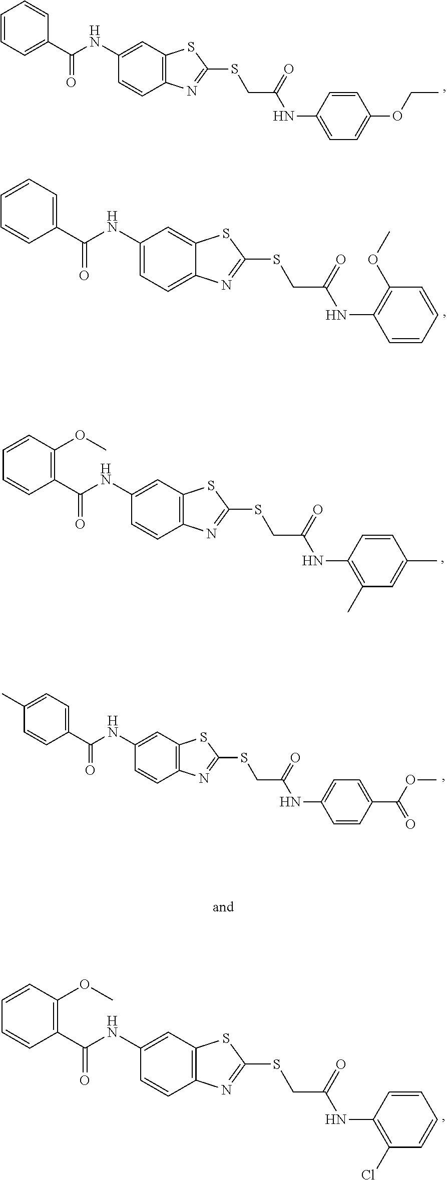
or a pharmaceutically acceptable salt of any one thereof.

37. A compound of Formula (II): 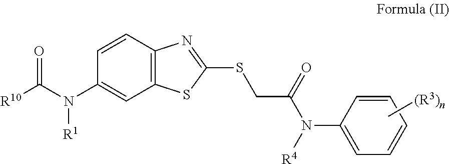
or a pharmaceutically acceptable salt thereof, wherein:
R¹ and R⁴ are each independently selected from hydrogen and C₁₋₆ alkyl;
R¹⁰ is selected from 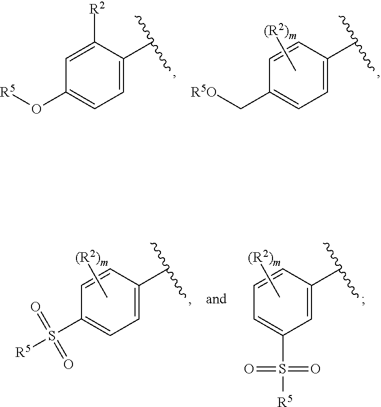
each R² and R³ is independently selected from halogen, —OR⁵, —SR⁵, —N(R⁵)₂, —C(O)R⁶, —C(O)N(R⁵)₂, —N(R⁵)C(O)R⁶, —C(O)OR⁵, —C(O)OR⁵, —OC(O)R⁶, —S(O)₂R⁶, —S(O)₂N(R⁵)₂, —NO₂, —CN, C₁₋₆ alkyl, C₂₋₆ alkenyl, and C₂₋₆ alkynyl; wherein each C₁₋₆ alkyl, C₂₋₆ alkenyl, and C₂₋₆ alkynyl is optionally substituted with one or more substituents independently selected from halogen, —OR⁵, —SR⁵, —N(R⁵)₂, —C(O)R⁶, —C(O)N(R⁵)₂, —N(R⁵)C(O)R⁶—C(O)OR⁵, —OC(O)R⁶, —NO₂, ═O, and —CN;
each R⁵ is independently selected from hydrogen; and C₁₋₆ alkyl optionally substituted with one more substituents independently selected from halogen, —O—C₁₋₆ alkyl, —O—C₁₋₆ haloalkyl, —NH₂—NO₂, ═O, and —CN;
each R⁶ is independently selected from C₁₋₆ alkyl optionally substituted with one more substituents independently selected from halogen, —O—C₁₋₆ alkyl, —O—C₁₋₆ haloalkyl, —NH₂—NO₂, ═O, and —CN;
m is 0, 1, 2, 3, or 4; and
n is 0, 1, 2, 3, 4, or 5.

38. The compound or salt of claim 37, wherein
R¹ and R⁴ are each hydrogen;
m is selected from 0 and 1;
R¹⁰ is selected from 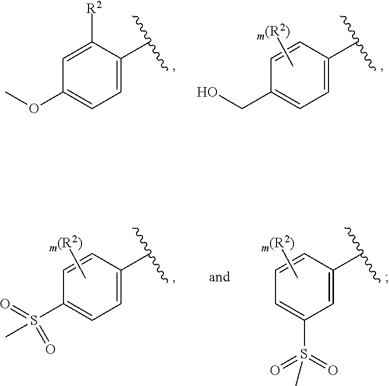
n is selected from 0 and 1; and
each R³ is independently halogen, and —S(O)₂N(R⁵)₂.

39. The compound or salt of claim 37, wherein
each m is selected from 0 and 1,
each R² is independently selected from the group consisting of halogen;
n is selected from 0 and 1; and
each R³ is independently halogen, and —S(O)₂NH₂.

40. The compound or salt of claim 37, wherein R¹⁰ is selected from

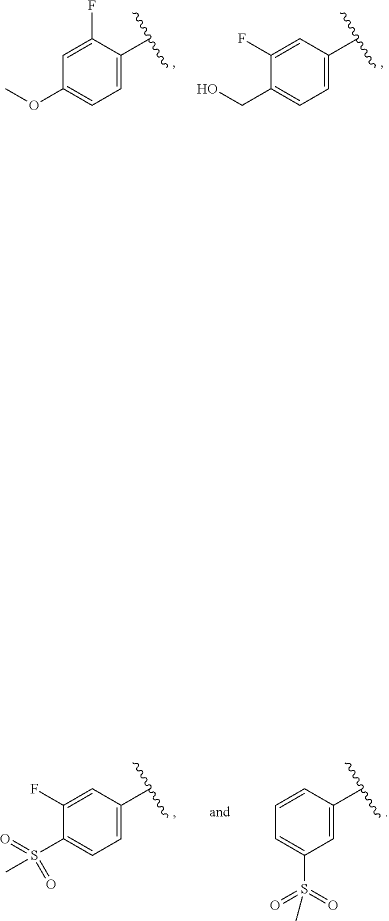

41. The compound or salt of claim 37, wherein the compound is selected from

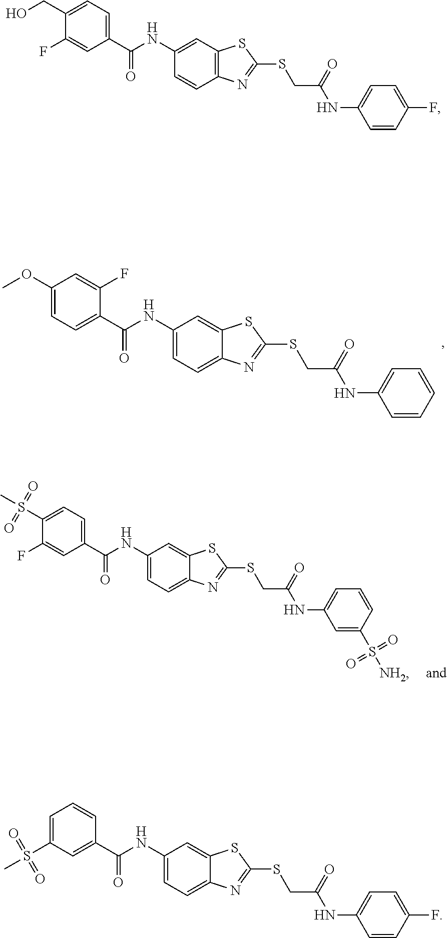 [SMILES: O=C(CSc1nc2ccc(NC(=O)c3ccc(CO)c(F)c3)cc2s1)Nc1ccc(F)cc1 ; COc1ccc(C(=O)Nc2ccc3nc(SCC(=O)Nc4ccccc4)sc3c2)c(F)c1 ; CS(=O)(=O)c1ccc(C(=O)Nc2ccc3nc(SCC(=O)Nc4cccc(S(N)(=O)=O)c4)sc3c2)cc1F ; CS(=O)(=O)c1cccc(C(=O)Nc2ccc3nc(SCC(=O)Nc4ccc(F)cc4)sc3c2)c1]

42. A pharmaceutical composition comprising a compound of claim 37 and a pharmaceutically acceptable excipient.

43. A method of treating a disease or disorder in a subject in need thereof, comprising administering to the subject a compound of claim 37.

44. The method of claim 43, wherein the disease or disorder is a senescence-associated disease or disorder.

45. A method of decreasing senescent cell burden and/or mRNA expression of senescence-associated genes in a subject comprising contacting the senescent cell with the compound or salt of claim 37.
